(19) United States
(12) Patent Application Publication
(10) Pub. No.: US 2017/0334927 A1
(43) Pub. Date: Nov. 23, 2017

(54) Nitroimidazole Compound, Preparation Method Therefor And Use Thereof In Drug Manufacturing

(71) Applicant: Shanghai Sun-sail Pharmaceutical Science & Technology Co., Ltd., Pudong New District Shanghai (CN)

(72) Inventors: Chuansheng Zhao, Pudong New District Shanghai (CN); Bingbin Zhang, Pudong New District Shanghai (CN); Tongrui Zhou, Pudong New District Shanghai (CN); Tiancai Wang, Pudong New District Shanghai (CN); Jun Sun, Pudong New District Shanghai (CN); Houxing Fan, Pudong New District Shanghai (CN)

(73) Assignee: Shanghai Sun-Sail Pharmaceutical Science & Technology Co., Ltd., Pudong New District Shanghai (CN)

(21) Appl. No.: 15/534,886

(22) PCT Filed: Dec. 11, 2015
(86) PCT No.: PCT/CN2015/097096

§ 371 Date: Jun. 9, 2017

(30) Foreign Application Priority Data
Dec. 12, 2014 (CN) 201410767127.6

(51) Int. Cl. C07D 498/04; C07D 519/00

(52) U.S. Cl. CPC C07D 498/04; C07D 519/00

(57) ABSTRACT

A nitroimidazole compound represented by general formula (I) or an optical isomer thereof or a pharmaceutically acceptable salt thereof, and a preparation method therefore, and use thereof in manufacturing drugs for the treating infectious diseases caused by mycobacterium tuberculosis. Specific groups in general formula (I) are as defined in the specification.

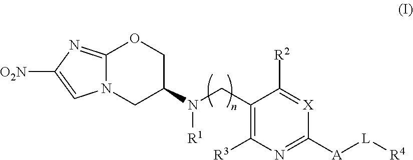

### FIELD OF THE INVENTION

The present invention falls within the fields of pharmacy, medicinal chemistry and pharmacology, and more specifically, relates to a novel class of nitroimidazole compounds, preparation methods therefor, and use of such compounds to treat diseases associated with infections caused by Mycobacterium tuberculosis.

### BACKGROUND OF THE INVENTION

Tuberculosis is caused by Mycobacterium tuberculosis infection, is one of the oldest diseases of mankind and still seriously endangers human health to date. According to WHO's statistics, about one in three people in the world had been infected with Mycobacterium tuberculosis, and tuberculosis is an infectious disease which leads to the largest number of deaths.

At present, the treatment for tuberculosis diseases mainly adopts approaches using several first-line drugs in combination, such as isoniazid, rifampicin, ethambutol and pyrazinamide. This treatment method has the following shortcomings: a long treatment cycle, usually taking not less than six months; more serious adverse effects, for example, rifampicin and isoniazid in combination may cause serious hepatotoxicity and ethambutol can cause optic nerve damages; and poor effects or even ineffectiveness for drug-resistant Mycobacterium tuberculosis, especially multidrug-resistant Mycobacterium tuberculosis (MDR-TB).

In view of the above situations, there is an urgent need to develop a novel anti-tuberculosis drug now. This novel drug should have the following advantages: effective for drug-resistant tuberculosis, especially multidrug-resistant tuberculosis; capable of being combined with the first-line anti-tuberculosis drugs currently used; and having ideal metabolic properties and capable of being administered orally.

WO 9701562 discloses many nitroimidazole compounds, in which a representative compound is PA-824, which has a new mechanism of action and can be used to treat tuberculosis. However, due to its low water solubility and low bioavailability, when administered orally, there are needs to formulate PA-824 into complex tablet formulations and further improve its anti-tuberculosis activity [Bioorg. Med. Chem. Lett, 2008, 18(7), 2256-2262].

OPC-67683 [J. Med. Chem., 2006, 49(26), 7854-7860] (Otsuka Pharmaceutical Co., Ltd.) has a mechanism of action similar to PA-824 and is used to treat tuberculosis. The compound was approved by the European Commission in May 2014 for the treatment of multidrug-resistant tuberculosis (MDR-TB) in adult patients. Although the compound has strong activity, it has the same problem as PA-824, i.e., the solubility of the compound in water is very poor, resulting in a very low oral bioavailability. Furthermore, PA-824 and OPC-67683 have very strong inhibition activity on hERG potassium channel, a side effect regarding to prolongation of QT-QTc interval and a serious cardiotoxicity safety issue clinically.

To this end, the object of the present invention is to provide a novel anti-tuberculosis nitroimidazole compound having no hERG inhibition activity, stronger antibacterial activity and improved water solubility to overcome the shortcomings currently existing in such compounds and develop a new generation of candidate drugs.

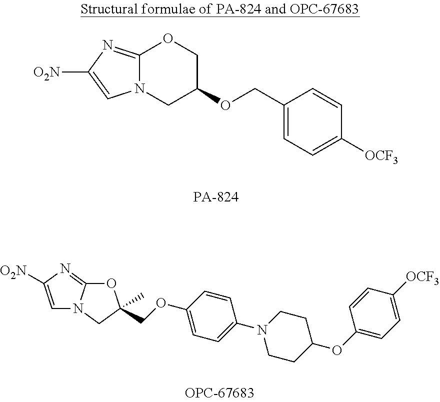 [SMILES: O=[N+]([O-])c1cn2c(n1)OC[C@@H](OCc1ccc(OC(F)(F)F)cc1)C2 ; C[C@]1(COc2ccc(N3CCC(Oc4ccc(OC(F)(F)F)cc4)CC3)cc2)Cn2cc([N+](=O)[O-])nc2O1]

### SUMMARY OF THE INVENTION

An object of the present invention is to provide a class of novel anti-tuberculosis compounds of general molecular formula as represented by (I) or optical isomers, pharmaceutically acceptable inorganic or organic salts thereof.

A second aspect of the present invention provides preparation methods for the compounds represented by formula (I) or various optical isomers, pharmaceutically acceptable inorganic or organic salts thereof.

A third aspect of the present invention provides use of the above-mentioned compounds of the present invention or various optical isomers, pharmaceutically acceptable inorganic or organic salts thereof in the manufacture of medicaments for the treatment of diseases caused by Mycobacterium tuberculosis infections, especially infectious diseases caused by multidrug-resistant Mycobacterium tuberculosis.

A fourth aspect of the present invention provides pharmaceutical compositions, comprising pharmacologically acceptable excipients or carriers and the compounds of formula (I) of the present invention or various optical isomers, pharmaceutically acceptable inorganic or organic salts thereof as active ingredients.

A first aspect of the present invention provides a class of novel nitroimidazole compounds, which are compounds of the following general formula (I) or optical isomers or pharmaceutically acceptable salts (inorganic or organic salts) thereof;

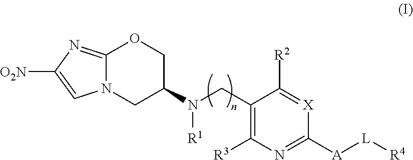

wherein in the general formula (I), n represents an integer between 1 and 4;

L is O, S, NH or a chemical bond;

X is C or N;

R¹ is hydrogen or C₁₋₆ alkyl;

R² and R³ can be the same or different and independently selected from hydrogen, halogen, cyano, trifluoromethyl, C₁₋₄ alkyl, C₃₋₆ cycloalkyl or C₁₋₄ alkoxy, respectively;

R⁴ is an aromatic ring or a heteroaromatic ring containing at least one heteroatom selected from N, O or S, wherein the aromatic ring or heteroaromatic ring is unsubstituted or substituted optionally with one to three groups independently selected from cyano, CF₃, OCF₃, halogen, methyl or methoxy; and

A can be selected from saturated or unsaturated C₅₋₇ cycloalkyl, C₈₋₁₀ fusedcycloalkyl, C₇₋₉ bridgedcycloalkyl or C₇₋₁₁ spirocycloalkyl, wherein the cycloalkyl has at least one carbon atom substituted with a nitrogen atom and is linked to the heteroaromatic ring (pyridine or pyrimidine) via the nitrogen atom and wherein the above-mentioned cycloalkyl can be substituted with one or more fluoro, cyano, hydroxyl, C₁₋₄ alkyl or C₁₋₄ alkoxy groups.

The pharmaceutically acceptable salts include salts formed by the compounds represented by the general formula (I) with acids, wherein the acids include inorganic acids, organic acids or acidic amino acids, wherein the inorganic acids include hydrochloric acid, hydrobromic acid, hydrofluoric acid, sulfuric acid, nitric acid or phosphoric acid, the organic acids include formic acid, acetic acid, propionic acid, oxalic acid, trifluoroacetic acid, malonic acid, succinic acid, fumaric acid, maleic acid, lactic acid, malic acid, tartaric acid, citric acid, picric acid, methanesulfonic acid, p-toluenesulfonic acid, ethanesulfonic acid or benzenesulfonic acid, and the acidic amino acids include aspartic acid or glutamic acid.

Unless otherwise specified, the following terms used in the specification and claims have the following meanings:

“Alkyl” refers to a saturated aliphatic hydrocarbon group, including straight and branched chain groups of 1 to 6 carbon atoms. Lower alkyl groups containing 1 to 4 carbon atoms are preferred, for example methyl, ethyl, propyl, 2-propyl, n-butyl, isobutyl and t-butyl.

“Cycloalkyl” refers to a 3- to 6-membered all-carbon monocyclic aliphatic hydrocarbon group, wherein in the group, one or more rings may contain one or more double bonds, but none of the rings has a completely conjugated π-electron system, for example cyclopropyl, cyclobutyl, cyclopentyl, cyclohexane and cyclohexadiene. More preferred are cyclopropyl and cyclobutyl.

“Alkoxy” refers to an alkyl group bonded to the remainder of the molecule via an ether oxygen atom. Representative alkoxy groups are those having 1 to 4 carbon atoms, such as methoxy, ethoxy, propoxy, isopropoxy, butoxy, isobutoxy, sec-butoxy and tert-butoxy. As used herein, “alkoxy” includes unsubstituted and substituted alkoxy groups, especially alkoxy substituted with one or more halogens. Preferred alkoxy groups are selected from OCH₃, OCF₃, CHF₂O, CF₃CH₂O, iPrO, nPrO, iBuO, cPrO, nBuO or tBuO.

“Aryl” refers to a group having at least one aromatic ring structure, i.e., an aromatic ring having a conjugated π-electron system, including carbocyclic aryl and heteroaryl.

“Halogen” refers to fluorine, chlorine, bromine or iodine.

“Chemical bond” is a general term of strong interaction forces between two or more adjacent atoms (or ions) within a pure molecule or a crystal.

The above-mentioned “C₈₋₁₀ fused-cycloalkyl” refers to a cycloalkyl with two rings sharing two ring atoms. For example:

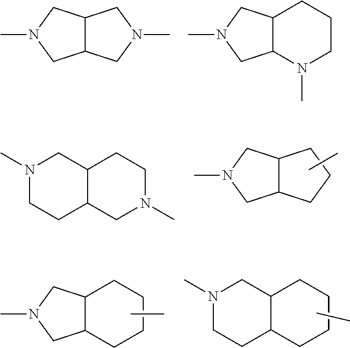

The above-mentioned structures are examples of a better understanding of the “fused-ring structure”, but not limitations on the “fused-ring structure”.

The above-mentioned “C₇₋₉ bridged-cycloalkyl” refers to a cycloalkyl with two rings sharing two or more ring atoms. For example,

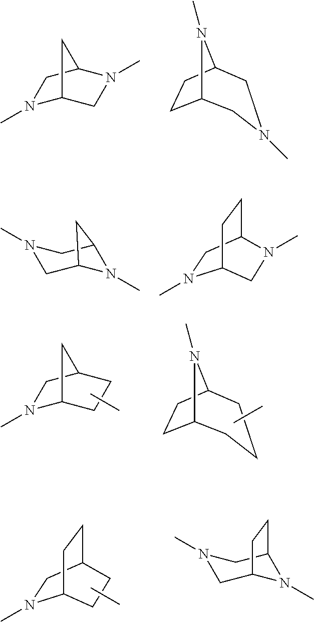

The above structures are examples of a better understanding of “bridged-cycloalkyl”, but not limitations on “bridged-cycloalkyl”.

The above-mentioned “C₇₋₁₁ spirocycloalkyl” refers to a cycloalkyl with two rings sharing one ring atom. For example:

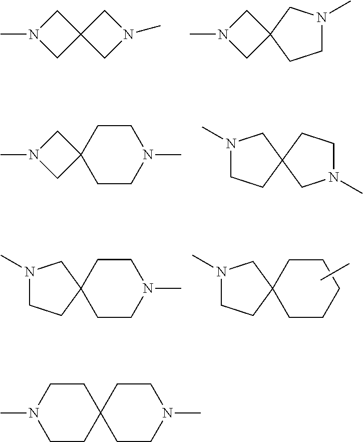

The above-mentioned structures are examples of a better understanding of “spirocycloalkyl”, but not limitations on “spirocycloalkyl”.

The compounds of the present invention may contain one or more asymmetric centers, and therefore appear in the form of racemate, racemic mixture, single enantiomer, diastereomeric compound and single diastereomer. The asymmetric centers which may exist depend on the nature of the various substituents on the molecule. Each of such asymmetric centers will independently produce two optical isomers, and all possible optical isomers and diastereomeric mixtures as well as pure or partially pure compounds are included within the scope of the present invention. The present invention is meant to include all such isomeric forms of these compounds.

The term “pharmaceutically acceptable salt” as used herein has no particular limitation as long as it is a pharmaceutically acceptable salt, including inorganic salts and organic salts. Specifically, salts formed by the compounds of the present invention with acids can be enumerated, wherein suitable salt-forming acids include, but are not limited to, inorganic acids such as hydrochloric acid, hydrobromic acid, hydrofluoric acid, sulfuric acid, phosphoric acid, nitric acid and phosphoric acid, organic acids such as formic acid, acetic acid, propionic acid, oxalic acid, trifluoroacetic acid, malonic acid, succinic acid, fumaric acid, maleic acid, lactic acid, malic acid, tartaric acid, citric acid, picric acid, methanesulfonic acid, benzenesulfonic acid and p-toluenesulfonic acid as well as acidic amino acids such as aspartic acid and glutamic acid.

The present inventors have synthesized and screened a large number of compounds after extensive research, and found for the first time that the compounds of formula (I) have strong inhibition activity against Mycobacterium tuberculosis and are particularly suitable for the preparation of medicaments for the treatment of diseases associated with infections caused by Mycobacterium tuberculosis. The present inventors have completed the present invention on this basis.

Preferably, in the compounds as represented by the structure of formula (I) of the present invention, the names and structural formulae of the representative compounds are shown in Table 1 below.

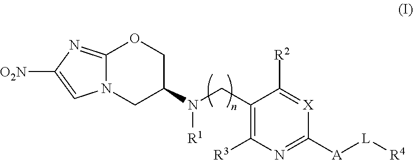

TABLE 1
Representative compounds of the present invention and structural
formulae thereof

| | Compound structure | Compound name |
|---|---|---|
| Compound 1 | 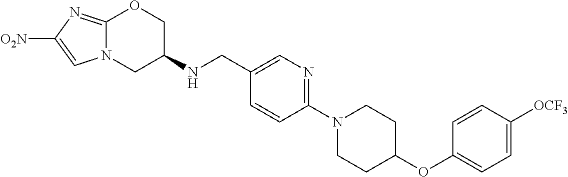 [SMILES: O=[N+]([O-])c1cn2c(n1)OC[C@@H](NCc1ccc(N3CCC(Oc4ccc(OC(F)(F)F)cc4)CC3)nc1)C2] | (S)-2-nitro-N-((6-(4-(4- (trifluoromethoxy)phenoxy) piperidin-1-yl)pyrid- 3-yl)methyl)-6,7-dihydro-5H- imidazo[2,1-b][1,3]oxazin- 6-amine |
| Compound 2 | 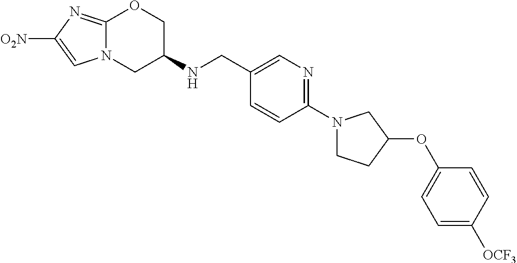 [SMILES: O=[N+]([O-])c1cn2c(n1)OC[C@@H](NCc1ccc(N3CCC(Oc4ccc(OC(F)(F)F)cc4)C3)nc1)C2] | (6S)-2-nitro-N-((6-(3-(4- (trifluoromethoxy)phenoxy) pyrrolidin-1-yl)pyrid-3- yl)methyl)-6,7-dihydro-5H- imidazo[2,1-b][1,3] oxazin-6-amine |
| Compound 3 | 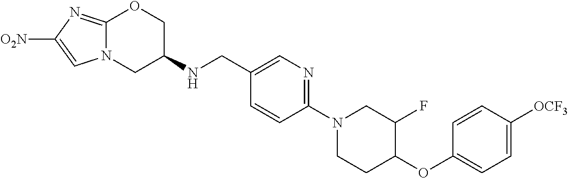 [SMILES: O=[N+]([O-])c1cn2c(n1)OC[C@@H](NCc1ccc(N3CCC(Oc4ccc(OC(F)(F)F)cc4)C(F)C3)nc1)C2] | (6S)-N-((6-(3-fluoro-4-(4- (trifluoromethoxy)phenoxy) piperidin-1-yl)pyrid-3- yl)methyl)-2-nitro-6,7- dihydro-5H-imidazo[2,1- b][1,3]oxazin-6-amine |
| Compound 4 | 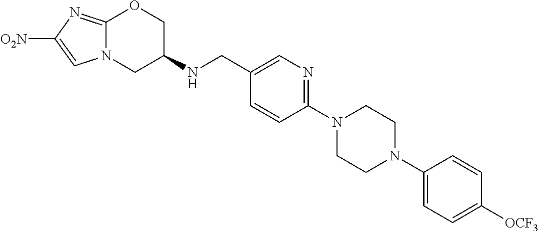 [SMILES: O=[N+]([O-])c1cn2c(n1)OC[C@@H](NCc1ccc(N3CCN(c4ccc(OC(F)(F)F)cc4)CC3)nc1)C2] | (S)-2-nitro-N-((6-(4-(4- (trifluoromethoxy)phenyl) piperazin-1-yl)pyrid-3- yl)methyl)-6,7-dihydro-5H- imidazo[2,1-b][1,3] oxazin-6-amine |
| Compound 5 | 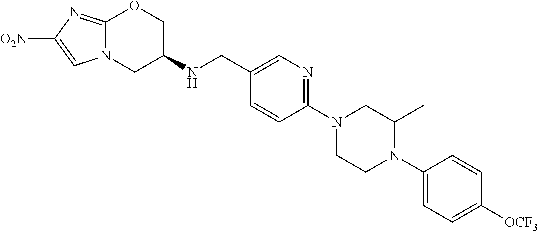 [SMILES: CC1CN(c2ccc(CN[C@@H]3COc4nc([N+](=O)[O-])cn4C3)cn2)CCN1c1ccc(OC(F)(F)F)cc1] | (3S)-N-((6-(3-methyl-4-(4- (trifluoromethoxy)phenyl) piperazin-1-yl)pyrid- 3-yl)methyl)-7-nitro-3,4- dihydro-2H-imidazo[2,1-b][1,3] oxazin-3-amine |
| Compound 6 | 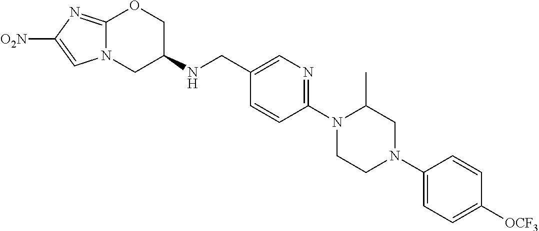 [SMILES: CC1CN(c2ccc(OC(F)(F)F)cc2)CCN1c1ccc(CN[C@@H]2COc3nc([N+](=O)[O-])cn3C2)cn1] | (3S)-N-((6-(2-methyl-4-(4- (trifluoromethoxy)phenyl) piperazin-1-yl)pyrid-3- yl)methyl)-7-nitro-3,4- dihydro-2H-imidazo[2,1-b][1,3] oxazin-3-amine |
| Compound 7 | 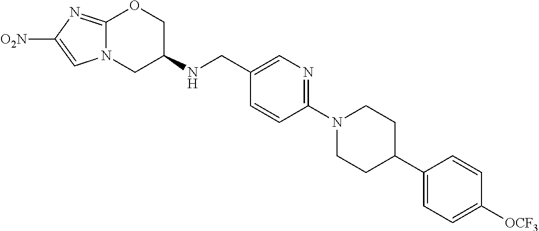 [SMILES: O=[N+]([O-])c1cn2c(n1)OC[C@@H](NCc1ccc(N3CCC(c4ccc(OC(F)(F)F)cc4)CC3)nc1)C2] | (S)-7-nitro-N-((6-(4-(4- (trifluoromethoxy)phenyl) piperidin-1-yl)pyrid- 3-yl)methyl)-3,4-dihydro-2H- imidazo[2,1-b][1,3]oxazin- 3-amine |
| Compound 8 | 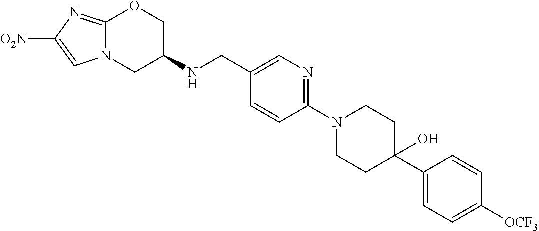 [SMILES: O=[N+]([O-])c1cn2c(n1)OC[C@@H](NCc1ccc(N3CCC(O)(c4ccc(OC(F)(F)F)cc4)CC3)nc1)C2] | (S)-1-(5-(((7-nitro-3,4-dihydro- 2H-imidazo[2,1-b][1,3]oxazin- 3-yl)amino)methyl)pyridin- 2-yl)-4-(4-(trifluoromethoxy) phenyl)piperidin-4-ol |
| Compound 9 | 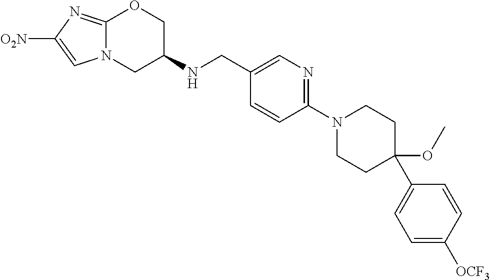 [SMILES: COC1(c2ccc(OC(F)(F)F)cc2)CCN(c2ccc(CN[C@@H]3COc4nc([N+](=O)[O-])cn4C3)cn2)CC1] | (S)-N-((6-(4-methoxy-4-(4- (trifluoromethoxy)phenyl) piperidin-1-yl)pyrid-3- yl)methyl)-7-nitro-3,4- dihydro-2H-imidazo[2,1- b][1,3]oxazin-3-amine |
| Compound 10 | 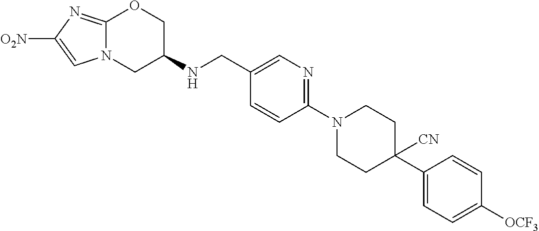 [SMILES: N#CC1(c2ccc(OC(F)(F)F)cc2)CCN(c2ccc(CN[C@@H]3COc4nc([N+](=O)[O-])cn4C3)cn2)CC1] | (S)-1-(5-(((7-nitro-3,4- dihydro-2H-imidazo[2,1- b][1,3]oxazin-3-yl) amino)methyl)pyridin-2- yl)-4-(4-(trifluoromethoxy) phenyl)piperidine- 4-carbonitrile |
| Compound 11 | 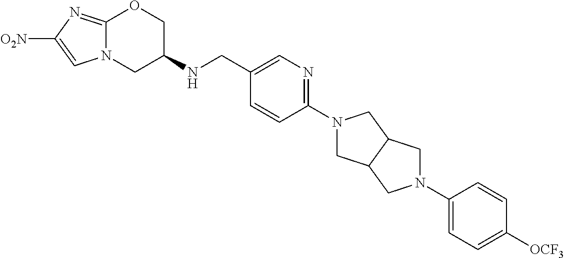 [SMILES: O=[N+]([O-])c1cn2c(n1)OC[C@@H](NCc1ccc(N3CC4CN(c5ccc(OC(F)(F)F)cc5)CC4C3)nc1)C2] | (6S)-2-nitro-N-((6-(5-(4- (trifluoromethoxy)phenyl) hexahydropyrrolo[3,4-c] pyrrol-2(1H)-yl)pyrid-3-yl) methyl)-6,7-dihydro-5H- imidazo[2,1-b][1,3]oxazin- 6-amine |
| Compound 12 | 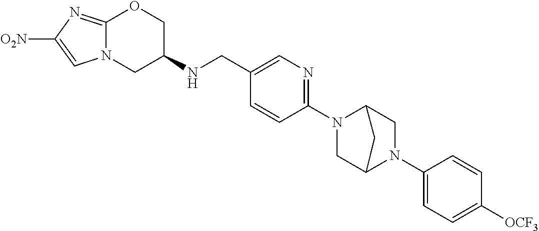 [SMILES: O=[N+]([O-])c1cn2c(n1)OC[C@@H](NCc1ccc(N3CC4CC3CN4c3ccc(OC(F)(F)F)cc3)nc1)C2] | (6S)-2-nitro-N-((6-(5-(4- (trifluoromethoxy)phenyl)-2,5- diazabicyclo[2.2.1]heptan- 2-yl)pyrid-3-yl)methyl)- 6,7-dihydro-5H-imidazo [2,1-b][1,3]oxazin-6-amine |
| Compound 13 | 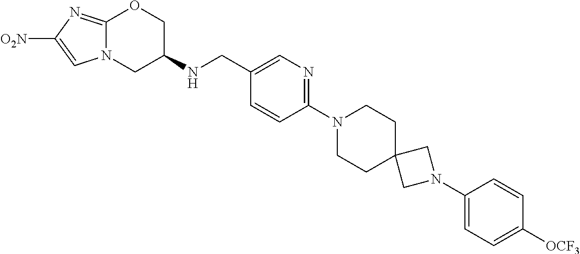 [SMILES: O=[N+]([O-])c1cn2c(n1)OC[C@@H](NCc1ccc(N3CCC4(CC3)CN(c3ccc(OC(F)(F)F)cc3)C4)nc1)C2] | (S)-2-nitro-N-((6-(2-(4- (trifluoromethoxy)phenyl)-2,7- diazaspiro[3.5]nonan-7-yl)pyrid- 3-yl)methyl)-6,7-dihydro-5H- imidazo[2,1-b][1,3] oxazin-6-amine |
| Compound 14 | 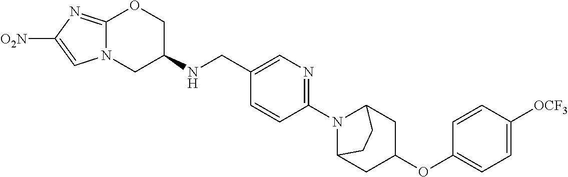 [SMILES: O=[N+]([O-])c1cn2c(n1)OC[C@@H](NCc1ccc(N3C4CCC3CC(Oc3ccc(OC(F)(F)F)cc3)C4)nc1)C2] | (6S)-2-nitro-N-((6-(3-(4- (trifluoromethoxy)phenoxy)- 8-azabicyclo[3.2.1]octan-8- yl)pyrid-3-yl)methyl)- 6,7-dihydro-5H-imidazo[2,1-b] [1,3]oxazin-6-amine |
| Compound 15 | 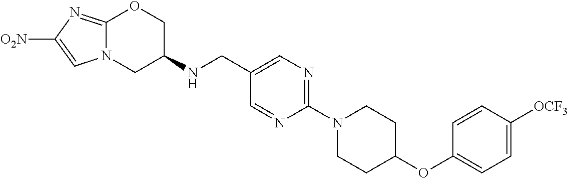 [SMILES: O=[N+]([O-])c1cn2c(n1)OC[C@@H](NCc1cnc(N3CCC(Oc4ccc(OC(F)(F)F)cc4)CC3)nc1)C2] | (S)-2-nitro-N-((6-(4-(4- (trifluoromethoxy)phenoxy) piperidin-1-yl)pyrimidin-3-yl) methyl)-6,7-dihydro- 5H-imidazo[2,1-b][1,3]oxazin- 6-amine |
| Compound 16 | 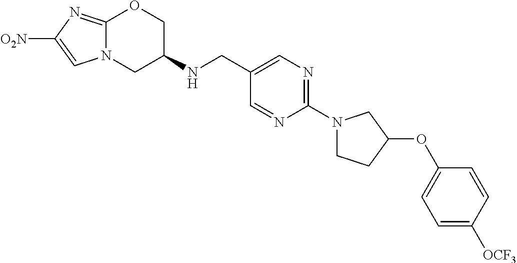 [SMILES: O=[N+]([O-])c1cn2c(n1)OC[C@@H](NCc1cnc(N3CCC(Oc4ccc(OC(F)(F)F)cc4)C3)nc1)C2] | (6S)-2-nitro-N-((6-(3-(4- (trifluoromethoxy)phenoxy) pyrrolidin-1-yl)pyrimidin- 3-yl)methyl)-6,7- dihydro-5H-imidazo[2,1- b][1,3]oxazin-6-amine |
| Compound 17 | 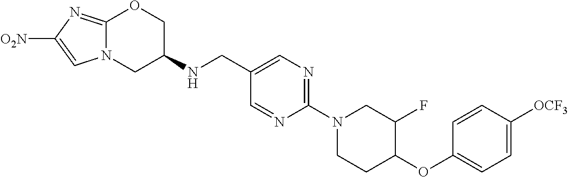 [SMILES: O=[N+]([O-])c1cn2c(n1)OC[C@@H](NCc1cnc(N3CCC(Oc4ccc(OC(F)(F)F)cc4)C(F)C3)nc1)C2] | (6S)-N-((6-(3-fluoro-4-(4- (trifluoromethoxy)phenoxy) piperidin-1-yl)pyrimidin-3- yl)methyl)-2-nitro-6,7- dihydro-5H-imidazo[2,1-b] [1,3]oxazin-6-amine |
| Compound 18 | 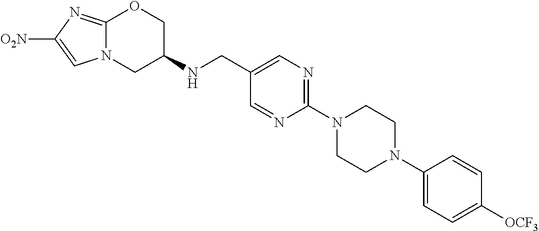 [SMILES: O=[N+]([O-])c1cn2c(n1)OC[C@@H](NCc1cnc(N3CCN(c4ccc(OC(F)(F)F)cc4)CC3)nc1)C2] | (S)-2-nitro-N-((6-(4-(4- (trifluoromethoxy)phenyl) piperazin-1-yl)pyrimidin- 3-yl)methyl)-6,7-dihydro- 5H-imidazo[2,1-b][1,3]oxazin- 6-amine |
| Compound 19 | 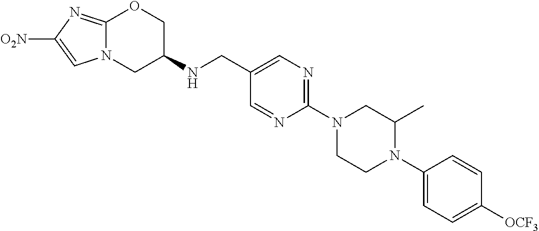 [SMILES: CC1CN(c2ncc(CN[C@@H]3COc4nc([N+](=O)[O-])cn4C3)cn2)CCN1c1ccc(OC(F)(F)F)cc1] | (3S)-N-((6-(3-methyl-4-(4- (trifluoromethoxy)phenyl) piperazin-1-yl)pyrimidin- 3-yl)methyl)-7-nitro-3,4- dihydro-2H-imidazo[2,1-b] [1,3]oxazin-3-amine |
| Compound 20 | 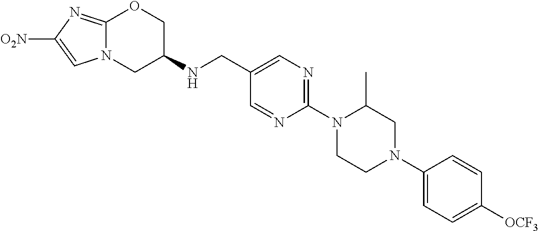 [SMILES: CC1CN(c2ccc(OC(F)(F)F)cc2)CCN1c1ncc(CN[C@@H]2COc3nc([N+](=O)[O-])cn3C2)cn1] | (3S)-N-((6-(2-methyl-4-(4- (trifluoromethoxy)phenyl) piperazin-1-yl)pyrimidin- 3-yl)methyl)-7-nitro-3,4- dihydro-2H-imidazo[2,1-b] [1,3]oxazin-3-amine |
| Compound 21 | 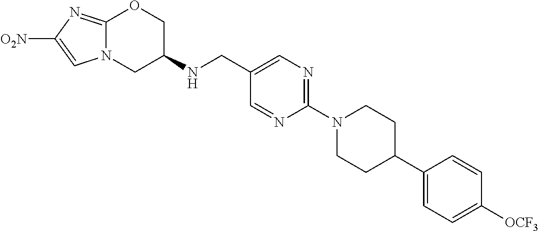 [SMILES: O=[N+]([O-])c1cn2c(n1)OC[C@@H](NCc1cnc(N3CCC(c4ccc(OC(F)(F)F)cc4)CC3)nc1)C2] | (S)-7-nitro-N-((6-(4-(4- (trifluoromethoxy)phenyl) piperidin-1-yl)pyrimidin- 3-yl)methyl)-3,4-dihydro- 2H-imidazo[2,1-b][1,3] oxazin-3-amine |
| Compound 22 | 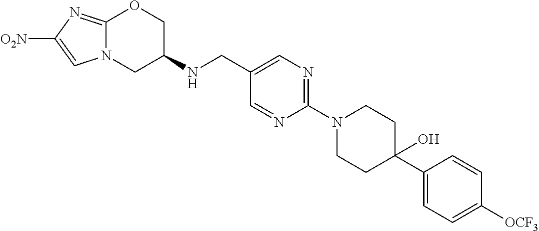 [SMILES: O=[N+]([O-])c1cn2c(n1)OC[C@@H](NCc1cnc(N3CCC(O)(c4ccc(OC(F)(F)F)cc4)CC3)nc1)C2] | (S)-1-(5-(((7-nitro-3,4-dihydro- 2H-imidazo[2,1-b][1,3]oxazin- 3-yl)amino)methyl)pyrimidin- 2-yl)-4-(4-(trifluoromethoxy) phenyl)piperidin-4-ol |
| Compound 23 | 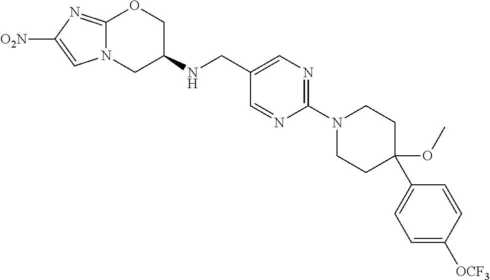 [SMILES: COC1(c2ccc(OC(F)(F)F)cc2)CCN(c2ncc(CN[C@@H]3COc4nc([N+](=O)[O-])cn4C3)cn2)CC1] | (S)-N-((6-(4-methoxy-4-(4- (trifluoromethoxy)phenyl) piperidin-1-yl)pyrimidin- 3-yl)methyl)-7-nitro-3,4- dihydro-2H-imidazo[2,1-b] [1,3]oxazin-3-amine |
| Compound 24 | 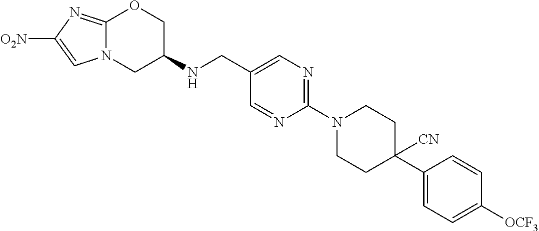 [SMILES: N#CC1(c2ccc(OC(F)(F)F)cc2)CCN(c2ncc(CN[C@@H]3COc4nc([N+](=O)[O-])cn4C3)cn2)CC1] | (S)-1-(5-(((7-nitro-3,4-dihydro-2H- imidazo[2,1-b][1,3]oxazin-3-yl) amino)methyl)pyrimidin-2-yl)-4- (4-(trifluoromethoxy)phenyl) piperidine-4-carbonitrile |
| Compound 25 | 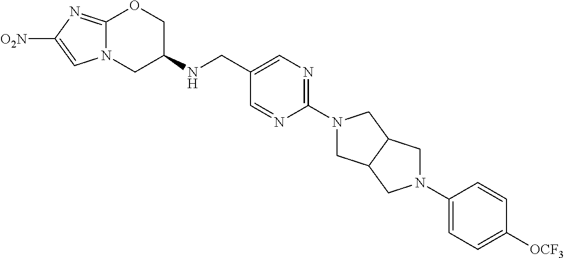 [SMILES: O=[N+]([O-])c1cn2c(n1)OC[C@@H](NCc1cnc(N3CC4CN(c5ccc(OC(F)(F)F)cc5)CC4C3)nc1)C2] | (6S)-2-nitro-N-((6-(5-(4- (trifluoromethoxy)phenyl) hexahydropyrrolo[3,4-c] pyrrol-2(1H)-yl)pyrimidin- 3-yl)methyl)-6,7-dihydro-5H- imidazo[2,1-b][1,3]oxazin- 6-amine |
| Compound 26 | 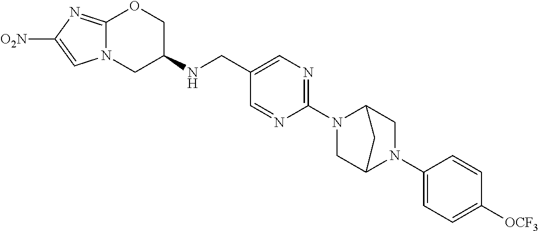 [SMILES: O=[N+]([O-])c1cn2c(n1)OC[C@@H](NCc1cnc(N3CC4CC3CN4c3ccc(OC(F)(F)F)cc3)nc1)C2] | (6S)-2-nitro-N-((6-(5-(4- (trifluoromethoxy)phenyl)- 2,5-diazabicyclo[2.2.1] heptan-2-yl)pyrimidin-3-yl) methyl)-6,7-dihydro-5H-imidazo [2,1-b][1,3]oxazin-6-amine |
| Compound 27 | 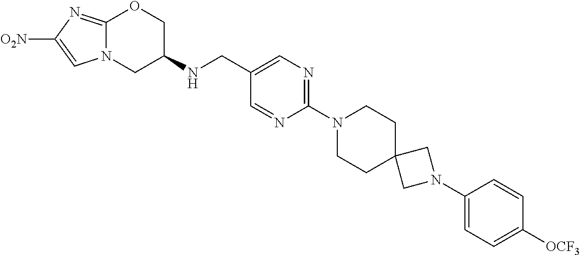 [SMILES: O=[N+]([O-])c1cn2c(n1)OC[C@@H](NCc1cnc(N3CCC4(CC3)CN(c3ccc(OC(F)(F)F)cc3)C4)nc1)C2] | (S)-2-nitro-N-((6-(2-(4- (trifluoromethoxy)phenyl)- 2,7-diazaspiro[3.5]nonan- 7-yl)pyrimidin-3-yl) methyl)-6,7-dihydro-5H- imidazo[2,1-b][1,3]oxazin- 6-amine |
| Compound 28 | 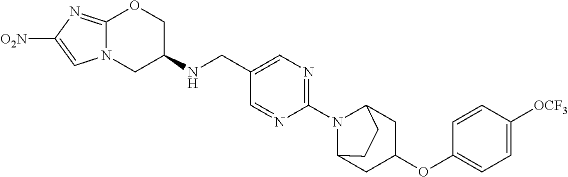 [SMILES: O=[N+]([O-])c1cn2c(n1)OC[C@@H](NCc1cnc(N3C4CCC3CC(Oc3ccc(OC(F)(F)F)cc3)C4)nc1)C2] | (6S)-2-nitro-N-((6-(3-(4- (trifluoromethoxy)phenoxy)- 8-azabicyclo[3.2.1]octan-8- yl)pyrimidin-3-yl) methyl)-6,7-dihydro-5H- imidazo[2,1-b][1,3]oxazin- 6-amine |
| Compound 29 | 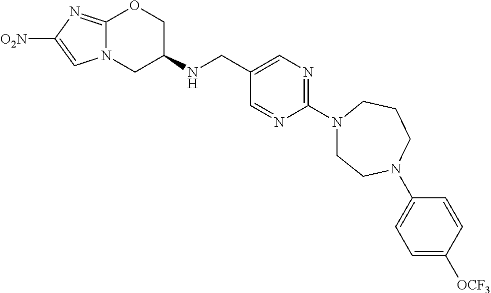 [SMILES: O=[N+]([O-])c1cn2c(n1)OC[C@@H](NCc1cnc(N3CCCN(c4ccc(OC(F)(F)F)cc4)CC3)nc1)C2] | (S)-2-nitro-N-((2-(4-(4- (trifluoromethoxy)phenyl)- 1,4-diazocyclohept-1-yl) pyrimidin-5-yl)methyl)-6,7- dihydro-5H-imidazo[2,1- b][1,3]oxazin-6-amine |
| Compound 30 | 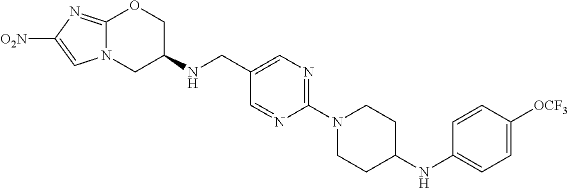 [SMILES: O=[N+]([O-])c1cn2c(n1)OC[C@@H](NCc1cnc(N3CCC(Nc4ccc(OC(F)(F)F)cc4)CC3)nc1)C2] | (S)-2-nitro-N-((2-(4-((4- (trifluoromethoxy)phenyl) amino)piperidin-1-yl)pyrimidin- 5-yl)methyl)-6,7-dihydro- 5H-imidazo[2,1-b][1,3] oxazin-6-amine |
| Compound 31 | 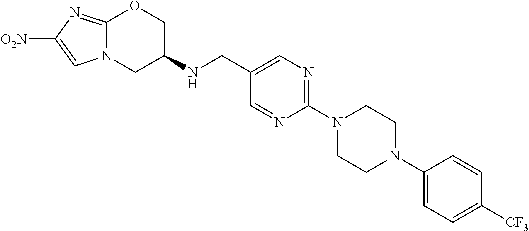 [SMILES: O=[N+]([O-])c1cn2c(n1)OC[C@@H](NCc1cnc(N3CCN(c4ccc(C(F)(F)F)cc4)CC3)nc1)C2] | (S)-2-nitro-N-((2-(4-(4- (trifluoromethyl)phenyl) piperazin-1-yl)pyrimidin- 5-yl)methyl)-6,7-dihydro- 5H-imidazo[2,1-b][1,3] oxazin-6-amine |
| Compound 32 | 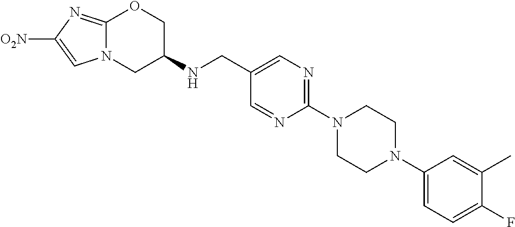 [SMILES: Cc1cc(N2CCN(c3ncc(CN[C@@H]4COc5nc([N+](=O)[O-])cn5C4)cn3)CC2)ccc1F] | (S)-N-((2-(4-(4-fluoro-3- methylphenyl)piperazin- 1-yl)pyrimidin-5-yl) methyl)-2-nitro-6,7-dihydro- 5H-imidazo[2,1-b][1,3] oxazin-6-amine |
| Compound 33 | 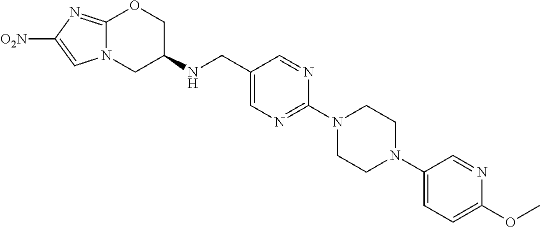 [SMILES: COc1ccc(N2CCN(c3ncc(CN[C@@H]4COc5nc([N+](=O)[O-])cn5C4)cn3)CC2)cn1] | (S)-N-((2-(4-(6-methoxypyridin- 3-yl)piperazin-1-yl)pyrimidin- 5-yl)methyl)-2-nitro-6,7- dihydro-5H-imidazo[2,1- b][1,3]oxazin-6-amine |
| Compound 34 | 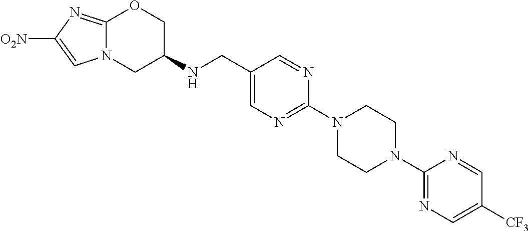 [SMILES: O=[N+]([O-])c1cn2c(n1)OC[C@@H](NCc1cnc(N3CCN(c4ncc(C(F)(F)F)cn4)CC3)nc1)C2] | (S)-2-nitro-N-((2-(4-(5- (trifluoromethyl)pyrimidin- 2-yl)piperazin-1-yl)pyrimidin- 5-yl)methyl)-6,7-dihydro- 5H-imidazo[2,1-b][1,3] oxazin-6-amine |
| Compound 35 | 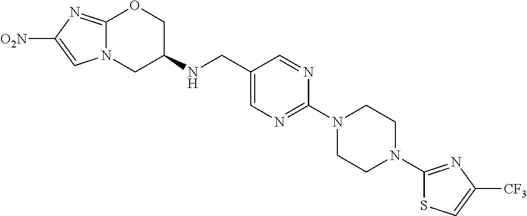 [SMILES: O=[N+]([O-])c1cn2c(n1)OC[C@@H](NCc1cnc(N3CCN(c4nc(C(F)(F)F)cs4)CC3)nc1)C2] | (S)-2-(4-(5-(((2-nitro-6,7- dihydro-5H-imidazo[2,1-b][1,3] oxazin-6-yl)amino)methyl) pyrimidin-2-yl)piperazin- 1-yl)thiazole-4-carbonitrile |
| Compound 36 | 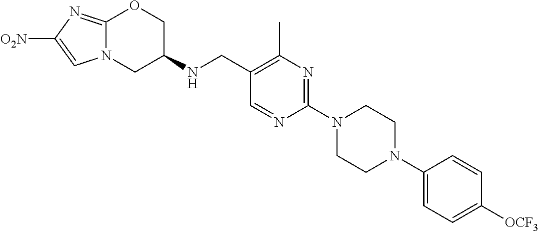 [SMILES: Cc1nc(N2CCN(c3ccc(OC(F)(F)F)cc3)CC2)ncc1CN[C@@H]1COc2nc([N+](=O)[O-])cn2C1] | (S)-N-((4-methyl-2-(4-(4- (trifluoromethoxy)phenyl) piperazin-1-yl) pyrimidin-5-yl)methyl)- 2-nitro-6,7-dihydro-5H- imidazo[2,1-b][1,3] oxazin-6-amine |
| Compound 37 | 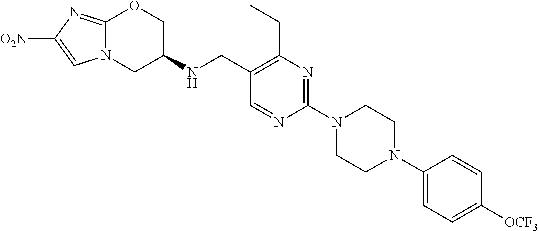 [SMILES: CCc1nc(N2CCN(c3ccc(OC(F)(F)F)cc3)CC2)ncc1CN[C@@H]1COc2nc([N+](=O)[O-])cn2C1] | (S)-N-((4-methyl-2-(4-(4- (trifluoromethoxy)phenyl) piperazin-1-yl)pyrimidin- 5-yl)ethyl)-2-nitro-6,7- dihydro-5H-imidazo[2,1- b][1,3]oxazin-6-amine |
| Compound 38 | 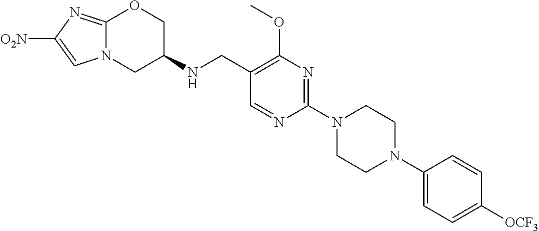 [SMILES: COc1nc(N2CCN(c3ccc(OC(F)(F)F)cc3)CC2)ncc1CN[C@@H]1COc2nc([N+](=O)[O-])cn2C1] | (S)-N-((4-methoxy-2-(4-(4- (trifluoromethoxy)phenyl) piperazin-1-yl)pyrimidin- 5-yl)methyl)-2-nitro-6,7- dihydro-5H-imidazo[2,1- b][1,3]oxazin-6-amine |
| Compound 39 | 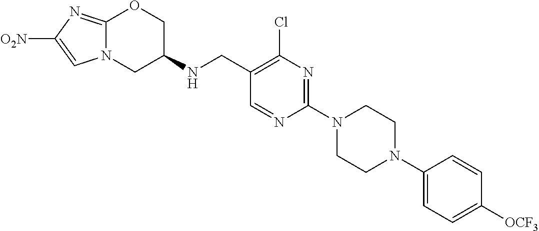 [SMILES: O=[N+]([O-])c1cn2c(n1)OC[C@@H](NCc1cnc(N3CCN(c4ccc(OC(F)(F)F)cc4)CC3)nc1Cl)C2] | (S)-N-((4-chloro-2-(4-(4- (trifluoromethoxy)phenyl) piperazin-1-yl)pyrimidin- 5-yl)methyl)-2-nitro-6,7- dihydro-5H-imidazo[2,1-b][1,3] oxazin-6-amine |
| Compound 40 | 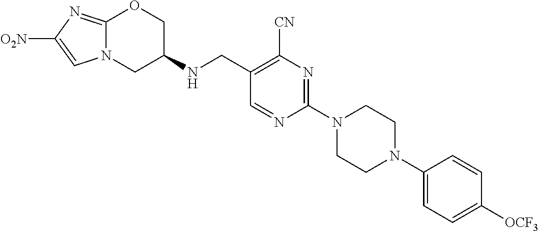 [SMILES: N#Cc1nc(N2CCN(c3ccc(OC(F)(F)F)cc3)CC2)ncc1CN[C@@H]1COc2nc([N+](=O)[O-])cn2C1] | (S)-5-(((2-nitro-6,7-dihydro- 5H-imidazo[2,1-b][1,3]oxazin- 6-yl)amino)methyl)-2-(4-(4- (trifluoromethoxy) phenyl)piperazin-1-yl) pyrimidine-4-carbonitrile |
| Compound 41 | 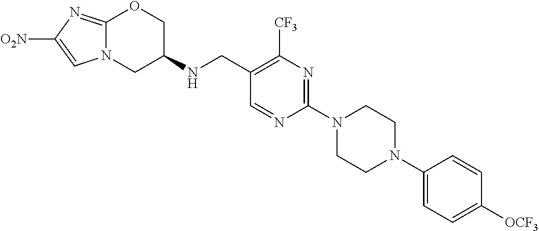 [SMILES: O=[N+]([O-])c1cn2c(n1)OC[C@@H](NCc1cnc(N3CCN(c4ccc(OC(F)(F)F)cc4)CC3)nc1C(F)(F)F)C2] | (S)-2-nitro-N-((2-(4-(4- (trifluoromethoxy)phenyl) piperazin-1-yl)-4- (trifluoromethyl)pyrimidin- 5-yl)methyl)-6,7-dihydro- 5H-imidazo[2,1-b][1,3] oxazin-6-amine |
| Compound 42 | 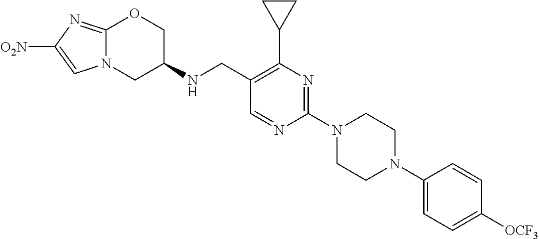 [SMILES: O=[N+]([O-])c1cn2c(n1)OC[C@@H](NCc1cnc(N3CCN(c4ccc(OC(F)(F)F)cc4)CC3)nc1C1CC1)C2] | (S)-N-((4-cyclopropyl-2-(4- (4-(trifluoromethoxy)phenyl) piperazin-1-yl)pyrimidin-5- yl)methyl)-2-nitro- 6,7-dihydro-5H-imidazo[2,1- b][1,3]oxazin-6-amine |
| Compound 43 | 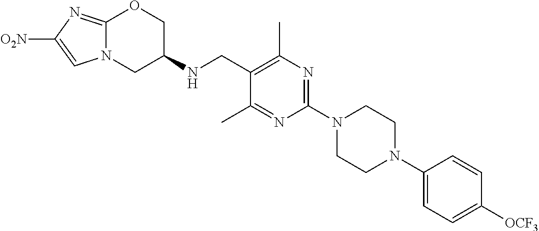 [SMILES: Cc1nc(N2CCN(c3ccc(OC(F)(F)F)cc3)CC2)nc(C)c1CN[C@@H]1COc2nc([N+](=O)[O-])cn2C1] | (S)-N-((4,6-dimethyl-2-(4-(4- (trifluoromethoxy)phenyl) piperazin-1-yl)pyrimidin- 5-yl)methyl)-2-nitro- 6,7-dihydro-5H-imidazo [2,1-b][1,3]oxazin-6-amine |
| Compound 44 | 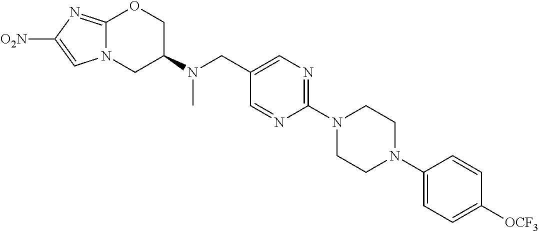 [SMILES: CN(Cc1cnc(N2CCN(c3ccc(OC(F)(F)F)cc3)CC2)nc1)[C@@H]1COc2nc([N+](=O)[O-])cn2C1] | (S)-N-methyl-2-nitro-N-((2- (4-(4-(trifluoromethoxy) phenyl)piperazin-1- yl)pyrimidin-5-yl)methyl)- 6,7-dihydro-5H-imidazo [2,1-b][1,3]oxazin-6-amine |
| Compound 45 | 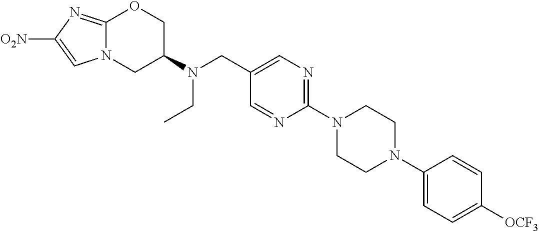 [SMILES: CCN(Cc1cnc(N2CCN(c3ccc(OC(F)(F)F)cc3)CC2)nc1)[C@@H]1COc2nc([N+](=O)[O-])cn2C1] | (S)-N-ethyl-2-nitro-N-((2-(4- (4-(trifluoromethoxy)phenyl) piperazin-1-yl)pyrimidin-5- yl)methyl)-6,7-dihydro- 5H-imidazo[2,1-b][1,3] oxazin-6-amine |
| Compound 46 | 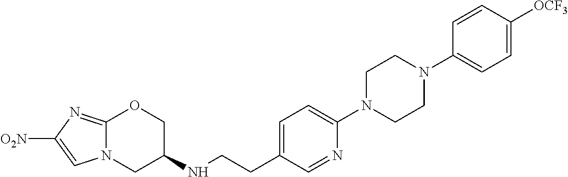 [SMILES: O=[N+]([O-])c1cn2c(n1)OC[C@@H](NCCc1ccc(N3CCN(c4ccc(OC(F)(F)F)cc4)CC3)nc1)C2] | (S)-2-nitro-N-(2-(6-(4-(4- (trifluoromethoxy)phenyl) piperazin-1-yl)pyrid-3-yl)ethyl)- 6,7-dihydro-5H-imidazo[2,1-b] [1,3]oxazin-6-amine |
| Compound 47 | 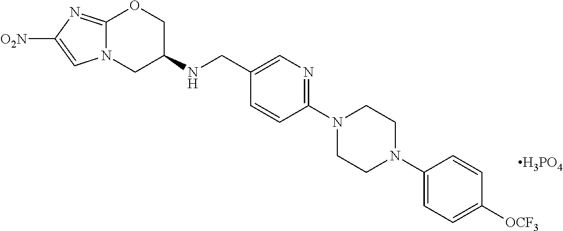 | (S)-2-nitro-N-((6-(4-(4- (trifluoromethoxy)phenyl) piperazin-1-yl) pyrid-3-yl)methyl)-6,7- dihydro-5H-imidazo [2,1-b][1,3]oxazin-6-amine phosphate |
| Compound 48 | 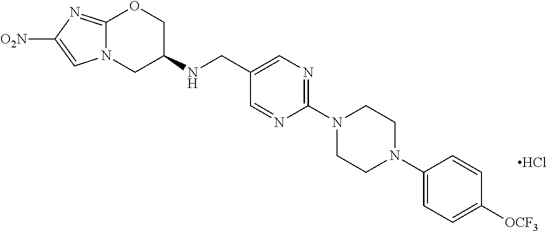 [SMILES: O=[N+]([O-])c1cn2c(n1)OC[C@@H](NCc1cnc(N3CCN(c4ccc(OC(F)(F)F)cc4)CC3)nc1)C2 ; Cl] | (S)-2-nitro-N-((2-(4-(4- (trifluoromethyl)phenyl) piperazin-1-yl) pyrimidin-5-yl)methyl)- 6,7-dihydro-5H- imidazo[2,1-b][1,3]oxazin-6- amine hydrochloride |
| Compound 49 | 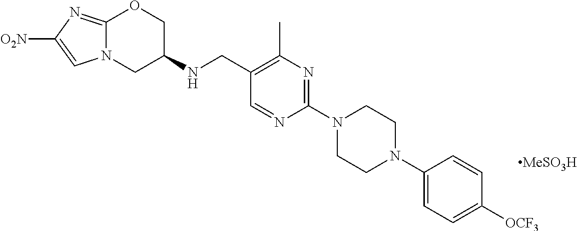 [SMILES: "·MeSO3H" ; Cc1nc(N2CCN(c3ccc(OC(F)(F)F)cc3)CC2)ncc1CN[C@@H]1COc2nc([N+](=O)[O-])cn2C1] | (S)-N-((4-methyl-2-(4-(4- (trifluoromethoxy)phenyl) piperazin-1-yl) pyrimidin-5-yl)methyl)- 2-nitro-6,7-dihydro-5H- imidazo[2,1-b][1,3]oxazin-6- amine methanesulfonate |
| Compound 50 | 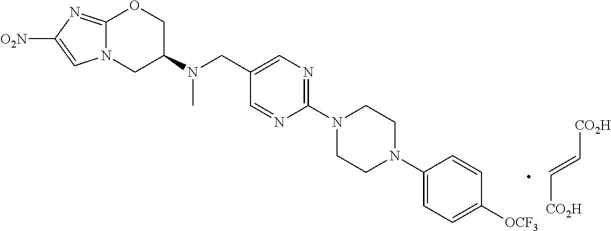 | (S)-N-methyl-2-nitro-N-((2-(4-(4- (trifluoromethoxy)phenyl) piperazin-1-yl)pyrimidin-5-yl) methyl)-6,7-dihydro-5H-imidazo[2,1- b][1,3]oxazin-6-amine fumarate |

A second aspect of the present invention provides preparation methods for the above-mentioned novel nitroimidazole compounds or pharmaceutically acceptable inorganic or organic salts thereof.

The preparation methods for the compounds represented by the structure of the general formula (I) of the present invention will be described in detail below, but these specific methods do not set any limit to the present invention.

The compounds represented by the structure of the general formula (I) of the present invention can be prepared by the following methods; however, the conditions of the methods, such as reactants, solvents, bases, amounts of the compounds used, reaction temperatures, times required for the reactions and the like are not limited to the following explanations. The compounds of the present invention may also be conveniently prepared by optionally combining various synthetic methods described in the present specification or known in the art, and such combinations may be readily carried out by those skilled in the art to which the present invention pertains.

The schemes of the preparation methods for the anti-bacterial nitroimidazole compounds of the present invention can include:

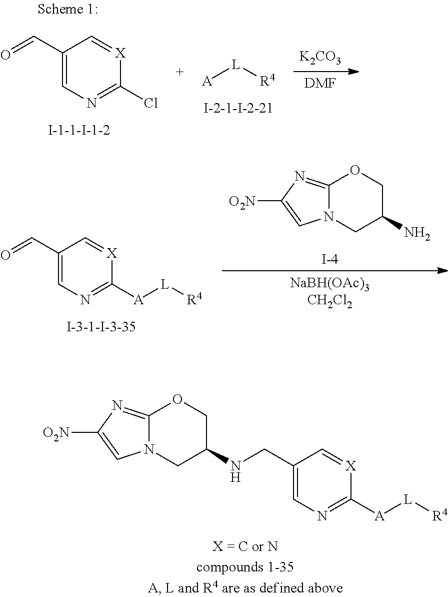

(1) Raw materials I-1-1-I-1-2 and I-2-1-I-2-21 were subjected to a substitution reaction for 1-24 hours in a solvent at 20° C. to 150° C. or solvent reflux temperature under alkaline conditions, giving intermediates I-3-1-I-3-35.

In step (1), the solvent can be selected from such solvents as acetonitrile, acetone, dioxane, tetrahydrofuran, methanol, ethanol, isopropanol, dimethylformamide, dimethylacetamide, ethylene glycol dimethyl ether, dimethylsulfoxide and water and can be a single solvent or a mixed solvent.

In step (1), the base can be selected from sodium hydroxide, potassium hydroxide, lithium hydroxide, barium hydroxide, potassium carbonate, sodium carbonate, cesium carbonate, sodium bicarbonate, potassium bicarbonate, potassium tert-butoxide, sodium tert-butoxide, sodium hydride, potassium hydride, triethylamine, diisopropylethylamine and the like. The optimal reaction conditions were as follows: reacting raw materials I-1-1-I-1-2 with I-2-1-I-2-21 for 2-12 hours at 120° C. using dimethylformamide (DMF) as the solvent and potassium carbonate as the base.

(2) Intermediates I-3-1-I-3-35 were reacted with amine 1-4 (reference: J. Med. Chem. 2009, 52(5), 1329-1344) in a solvent under alkaline conditions to form an imine intermediate state which was then subjected to a reductive amination reaction for 1-24 hours in the presence of a reducing agent, giving compounds 1-35.

In step (2), the solvent can be selected from methanol, ethanol, isopropanol, tetrahydrofuran, dichloromethane, 1,2-dichloroethane, dioxane, dimethylformamide, acetonitrile, ethylene glycol dimethyl ether, water and the like and can be a single solvent or a mixed solvent.

In step (2), the base can be selected from pyridine, triethylamine, diisopropylethylamine and other organic bases. The reducing agent is selected from sodium borohydride, potassium borohydride, sodium cyanoborohydride, sodium triacetoxyborohydride and the like. The optimal reaction conditions were as follows: reacting intermediates I-3-1-I-3-35 with amine 1-4 at room temperature to form an imine firstly using dichloromethane as the solvent and triethylamine as the base, which was then reduced with sodium triacetoxyborohydride and reacted for a further 4-16 hours at room temperature.

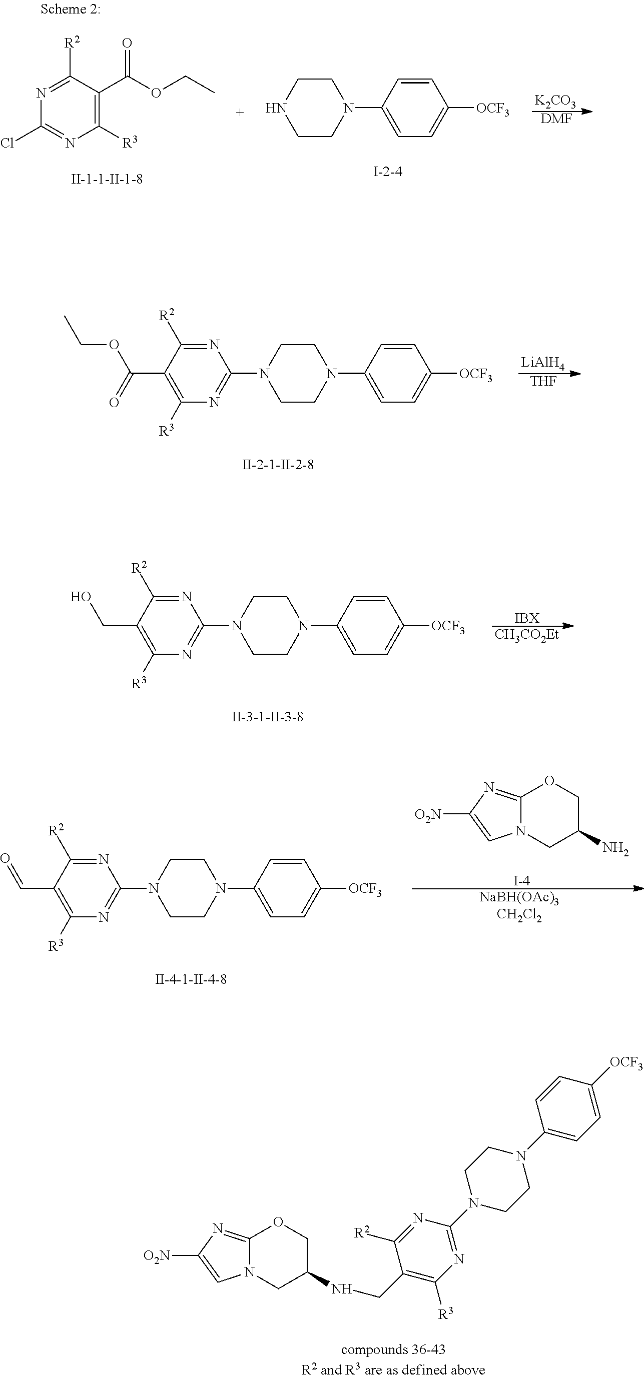

(1) Raw materials II-1-1-II-1-8 and I-2-4 (reference: WO 2003/105853 A1) were subjected to a substitution reaction for 1-24 hours in a solvent at 20° C. to 150° C. or solvent reflux temperature, giving intermediates II-2-1-I-2-8.

In step (1), the solvent can be selected from such solvents as acetonitrile, acetone, dioxane, tetrahydrofuran, methanol, ethanol, isopropanol, dimethylformamide, dimethylacetamide, ethylene glycol dimethyl ether, dimethylsulfoxide and water and can be a single solvent or a mixed solvent.

In step (1), the base can be selected from sodium hydroxide, potassium hydroxide, lithium hydroxide, barium hydroxide, potassium carbonate, sodium carbonate, cesium carbonate, sodium bicarbonate, potassium bicarbonate, potassium tert-butoxide, sodium tert-butoxide, sodium hydride, potassium hydride, triethylamine, diisopropylethylamine and the like. The optimal reaction conditions were as follows: reacting raw materials II-1-1-II-1-8 with I-2-4 for 2-12 hours at 90° C. using dimethylformamide as the solvent and potassium carbonate as the base.

(2) Intermediates II-2-1-II-2-8 were subjected to a reduction reaction for 0.5-24 hours in a solvent at −78° C. to 40° C., giving intermediates II-3-1-II-3-8.

In step (2), the solvent can be selected from such solvents as toluene, tetrahydrofuran, n-hexane, cyclohexane, methyltetrahydrofuran, diethyl ether, methyl tert-butyl ether, ethylene glycol dimethyl ether and water and can be a single solvent or a mixed solvent.

In step (2), the reducing agent can be selected from sodium borohydride, potassium borohydride, lithium borohydride, lithium aluminum hydride, diisobutylaluminum hydride, red aluminum and the like. The optimal reaction conditions were as follows: performing the reaction for 1-3 hours at −30° C. to 20° C. using anhydrous tetrahydrofuran as the solvent and lithium aluminum hydride as the reducing agent.

(3) Intermediates II-3-1-II-3-8 were subjected to an oxidation reaction for 1-24 hours in a solvent at 20° C. to 150° C. or solvent reflux temperature, giving intermediates II-4-1-II-4-8.

In step (3), the solvent can be selected from such solvents as ethyl acetate, dichloromethane, dioxane, tetrahydrofuran, trichloromethane, cyclohexane, dimethylformamide, dimethylacetamide, ethylene glycol dimethyl ether and dimethylsulfoxide and can be a single solvent or a mixed solvent.

In step (3), the oxidizing agent can be selected from active manganese dioxide, 2-iodacyl benzoic acid (IBX), Dess-Martin periodinane (DMP), pyridinium chlorochromate (PCC), pyridinium dichromate (PDC), pyridine sulfur trioxide, a mixed oxidizing agent of dimethylsulfoxide and oxalyl chloride (swern oxidation) or the like. The optimal reaction conditions were as follows: performing the reaction for 4-12 hours at 60° C. using anhydrous ethyl acetate as the solvent and IBX as the oxidizing agent.

(4) Intermediates II-4-1-II-4-8 were reacted with amine I-4 in a solvent under alkaline conditions to form an imine intermediate state which was then subjected to a reductive amination reaction for 1-24 hours in the presence of a reducing agent, giving compounds 36-43.

In step (4), the solvent can be selected from methanol, ethanol, isopropanol, tetrahydrofuran, dichloromethane, 1,2-dichloroethane, dioxane, dimethylformamide, acetonitrile, ethylene glycol dimethyl ether, water and the like and can be a single solvent or a mixed solvent.

In step (4), the base can be selected from pyridine, triethylamine, diisopropylethylamine and other organic bases. The reducing agent is selected from sodium borohydride, potassium borohydride, sodium cyanoborohydride, sodium triacetoxyborohydride and the like. The optimal reaction conditions were as follows: reacting intermediates II-4-1-II-4-8 with amine I-4 at room temperature to form an imine firstly using dichloromethane as the solvent and triethylamine as the base, which was then reduced with sodium triacetoxyborohydride and reacted for a further 4-16 hours at room temperature.

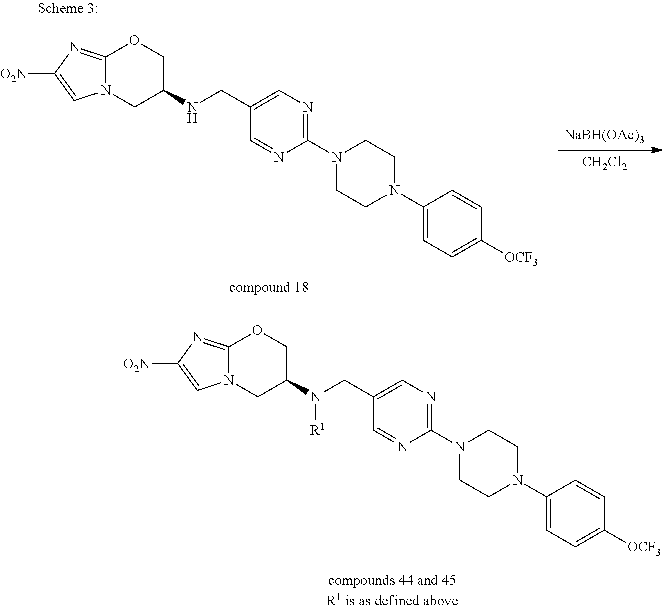

Compound 18 was reacted with different aldehydes in a solvent under acidic conditions to form an imine intermediate state which was then subjected to a reductive amination reaction for 1-24 hours in the presence of a reducing agent, giving compounds 44 and 45. The solvent can be selected from methanol, ethanol, isopropanol, tetrahydrofuran, dichloromethane, 1,2-dichloroethane, dioxane, dimethylformamide, acetonitrile, ethylene glycol dimethyl ether, water and the like and can be a single solvent or a mixed solvent.

The acid can be an organic weak acid or Lewis acid and selected from acetic acid, zinc chloride, zinc bromide, boron trifluoride diethyl etherate and the like. The reducing agent is selected from sodium borohydride, potassium borohydride, sodium cyanoborohydride, sodium triacetoxyborohydride and the like. The optimal reaction conditions were as follows: reacting compound 18 with an aldehyde at room temperature to form an imine firstly using tetrahydrofuran as the solvent and acetic acid as the acid, which was then reduced with sodium triacetoxyborohydride and reacted for a further 4-16 hours at room temperature.

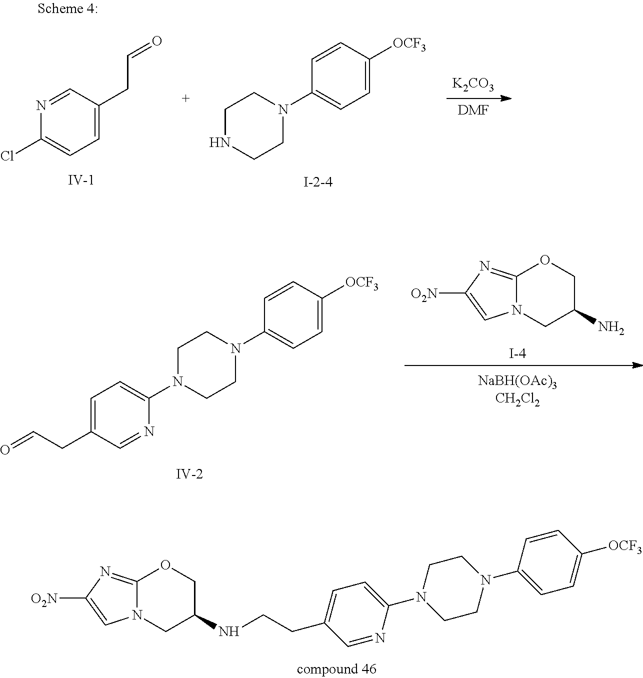

(1) Raw materials IV-1 (reference: Journal of the American Chemical Society, 2012, 134(30): 12466-12469) and I-2-4 were subjected to a substitution reaction for 1-24 hours in a solvent at 20° C. to 150° C. or solvent reflux temperature under alkaline conditions, giving intermediate IV-2.

In step (1), the solvent can be selected from such solvents as acetonitrile, acetone, dioxane, tetrahydrofuran, methanol, ethanol, isopropanol, dimethylformamide, dimethylacetamide, ethylene glycol dimethyl ether, dimethylsulfoxide and water and can be a single solvent or a mixed solvent.

In step (1), the base can be selected from sodium hydroxide, potassium hydroxide, lithium hydroxide, barium hydroxide, potassium carbonate, sodium carbonate, cesium carbonate, sodium bicarbonate, potassium bicarbonate, potassium tert-butoxide, sodium tert-butoxide, sodium hydride, potassium hydride, triethylamine, diisopropylethylamine and the like. The optimal reaction conditions were as follows: reacting raw materials IV-1 with 1-2-4 for 2-12 hours at 120° C. using dimethylformamide as the solvent and potassium carbonate as the base.

(2) Intermediate IV-2 was reacted with amine I-4 in a solvent under alkaline conditions to form an imine intermediate state which was then subjected to a reductive amination reaction for 1-24 hours in the presence of a reducing agent, giving compound 46.

In step (2), the solvent can be selected from methanol, ethanol, isopropanol, tetrahydrofuran, dichloromethane, 1,2-dichloroethane, dioxane, dimethylformamide, acetonitrile, ethylene glycol dimethyl ether, water and the like and can be a single solvent or a mixed solvent.

In step (2), the base can be selected from pyridine, triethylamine, diisopropylethylamine and other organic bases. The reducing agent is selected from sodium borohydride, potassium borohydride, sodium cyanoborohydride, sodium triacetoxyborohydride and the like. The optimal reaction conditions were as follows: reacting intermediates IV-2 with amine I-4 at room temperature to form an imine firstly using dichloromethane as the solvent and triethylamine as the base, which was then reduced with sodium triacetoxyborohydride and reacted for a further 4-16 hours at room temperature.

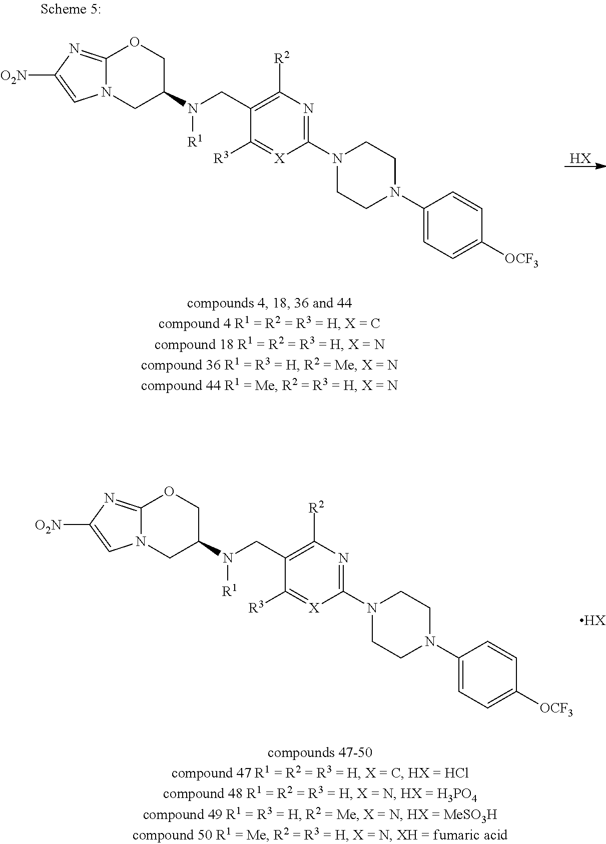

In a solvent, compound 4 was reacted with hydrochloric acid, compound 18 was reacted with phosphoric acid, compound 36 was reacted with methanesulfonic acid and compound 44 was reacted with fumaric acid respectively for 1-48 hours in a solvent under the conditions of −20° C. to 100° C. for direct precipitation of solids or static precipitation of solids or concentration and recrystallization, giving compounds 47-50.

The molar ratios of compound 4 to hydrochloric acid, compound 18 to phosphoric acid, compound 36 to methanesulfonic acid and compound 44 to fumaric acid are all preferably 1:1-1:10.

The solvent is selected from acetone, tetrahydrofuran, acetonitrile, ethanol, methanol, isopropanol, dichloromethane, 1,4-dioxane, dimethylformamide, dimethylacetamide, N-methylpyrrolidone, dimethylsulfoxide, water or the like and can be a single solvent or a mixed solvent.

The preferred conditions for the reaction were as follows: performing the reaction for 1-24 hours under the condition of room temperature using a mixed solution of dichloromethane and methanol with a volume ratio of 5:1-1:5 as the solvent.

A third aspect of the present invention provides use of the above-mentioned novel nitroimidazole compounds or pharmaceutically acceptable salts thereof in the manufacture of medicaments for the treatment of diseases associated with infections caused by Mycobacterium tuberculosis.

The compounds of the general formula (I) of the present invention have strong anti Mycobacterium tuberculosis effects, and in particular, have excellent effects on multidrug-resistant Mycobacterium tuberculosis.

The compounds of the general formula (I) of the present invention have increased water solubility, and drug metabolism studies in animals have shown that the compounds of the present invention have excellent pharmacokinetic properties. This is important for the present compounds improve the anti Mycobacterium tuberculosis activity, improve efficacy, reduce side effects and save costs.

In the present invention, “active ingredient” refers to a compound represented by the general formula (I) and a pharmaceutically acceptable inorganic or organic salt of the compound of the general formula (I). The compounds of the present invention may contain one or more asymmetric centers, and therefore appear in the form of racemate, racemic mixture, single enantiomer, diastereomeric compound and single diastereomer. The asymmetric centers which may exist depend on the nature of the various substituents on the molecule. Each of such asymmetric centers will independently produce two optical isomers, and all possible optical isomers and diastereomeric mixtures as well as pure or partially pure compounds are included within the scope of the present invention. The present invention is meant to include all such isomeric forms of these compounds.

Further, if necessary, the compounds of the present invention can be reacted with a pharmaceutically acceptable acid in a polar protic solvent, such as methanol, ethanol and isopropanol, to produce a pharmaceutically acceptable salt. The pharmaceutically acceptable inorganic or organic acid can be hydrochloric acid, hydrobromic acid, hydrofluoric acid, sulfuric acid, nitric acid, phosphoric acid, formic acid, acetic acid, propionic acid, oxalic acid, malonic acid, succinic acid, fumaric acid, maleic acid, lactic acid, malic acid, tartaric acid, citric acid, picric acid, methanesulfonic acid, ethanesulfonic acid, p-toluenesulfonic acid, aspartic acid, glutamic acid or the like.

As used herein, the term “caused by Mycobacterium tuberculosis” refers to causing by Mycobacterium tuberculosis sensitive to a clinical tuberculosis drug, Mycobacterium tuberculosis resistant to a clinical drug, Mycobacterium tuberculosis resistant to a variety of clinical drugs and extensively drug-resistant Mycobacterium tuberculosis.

The terms “diseases caused by Mycobacterium tuberculosis infections” and “Mycobacterium tuberculosis infectious diseases” can be used interchangeably, and as used herein, both refer to tuberculosis, lymphatic tuberculosis, intestinal tuberculosis, bone tuberculosis, tuberculous pleurisy, tuberculous meningitis and the like.

Since the compounds of the present invention have excellent anti Mycobacterium tuberculosis activity, the compounds of the present invention and various crystal forms and pharmaceutically acceptable inorganic or organic salts thereof as well as pharmaceutical compositions comprising the compounds of the present invention as the main active ingredients can be used to treat diseases associated with Mycobacterium tuberculosis. According to the prior art, the compounds of the present invention can be used to treat tuberculosis and other infectious diseases.

The present invention also provides pharmaceutical compositions for treating diseases associated with infections caused by Mycobacterium tuberculosis, comprising a therapeutically effective amount of the above-mentioned nitroimidazole compounds and pharmaceutically acceptable excipients or carriers.

The pharmaceutical compositions of the present invention comprise the nitroimidazole compounds of the present invention in a safe and effective amount range and pharmaceutically acceptable excipients or carriers. “A safe and effective amount” means that the amount of a compound is sufficient to significantly improve the condition without causing serious side effects. Typically, the pharmaceutical compositions comprise 1-1000 mg of the compounds of the present invention/dose, preferably 5-500 mg of the compounds of the present invention/dose, and more preferably 10-200 mg of the compounds of the present invention/dose.

The compounds of the present invention and pharmaceutically acceptable salts thereof can be formulated into various formulations, which comprise the compounds of the present invention or pharmaceutically acceptable salts thereof in a safe and effective amount range and a pharmaceutically acceptable excipient or carrier. “A safe and effective amount” means that the amount of a compound is sufficient to significantly improve the condition without causing serious side effects. The safe and effective amount of a compound is determined depending on the age, condition, course of treatment of the subject and other specific circumstances.

“A pharmaceutically acceptable excipient or carrier” means that one or more compatible solid or liquid fillers or gelling substances that are suitable for use by humans and must have sufficient purity and sufficiently low toxicity. “Compatibility” refers herein to the fact that the individual components of a composition can be admixed with a compound of the present invention and therewith without significantly reducing the efficacy of the compound. Some examples of the pharmaceutically acceptable excipients or carriers are cellulose and its derivatives (e.g., sodium carboxymethylcellulose, sodium ethylcellulose and cellulose acetate), gelatin, talc, solid lubricants (e.g., stearic acid and magnesium stearate), calcium sulfate, vegetable oils (e.g., soybean oil, sesame oil, peanut oil and olive oil), polyols (e.g., propylene glycol, glycerol, mannitol and sorbitol), emulsifying agents (e.g., Tween®), wetting agents (e.g., sodium dodecyl sulfate), colorants, flavoring agents, stabilizers, antioxidants, preservatives, pyrogen-free water and the like.

When administered, the compounds of the present invention may be administered orally, rectally, parenterally (intravenously, intramuscularly or subcutaneously) or topically.

Solid dosage forms for oral administration include capsules, tablets, pills, powders and granules. In these solid dosage forms, the active compound is mixed with at least one conventional inert excipient (or carrier), such as sodium citrate or dicalcium phosphate, or with the following ingredients: (a) a filler or compatibilizer, for example starch, lactose, sucrose, glucose, mannitol and silicic acid; (b) a binder, for example hydroxymethylcellulose, alginate, gelatin, polyvinylpyrrolidone, sucrose and acacia; (c) a humectant, for example glycerol; (d) a disintegrating agent, for example agar, calcium carbonate, potato starch or tapioca starch, alginic acid, certain composite silicates and carbonic acid; (e) a slow solvent, for example paraffin; (f) an absorbent accelerator, for example quaternary amine compounds; (g) a wetting agent, for example cetyl alcohol and glyceryl monostearate; (h) an adsorbent, for example kaolin; and (i) a lubricant, for example talc, calcium stearate, magnesium stearate, solid polyethylene glycol, sodium dodecyl sulfate or a mixture thereof. In capsules, tablets and pills, these dosage forms may also comprise buffering agents.

Solid dosage forms (e.g., tablets, dragees, capsules, pills and granules) can be prepared using coatings and shell materials, such as casings and other materials commonly known in the art. They may comprise an opacifying agent, and the release of the active compound or compound in such a composition may be achieved within a part of the digestive tract in a delayed manner. Examples of embedding components that may be used are polymeric materials and waxy materials. If desired, the active compound may also be mixed with one or more of the above-mentioned excipients to form microcapsules.

Liquid dosage forms for oral administration include pharmaceutically acceptable emulsions, solutions, suspensions, syrups or tinctures. In addition to the active compound, the liquid dosage form may comprise an inert diluent, such as water or other solvents, a solubilizer and an emulsifying agent conventionally used in the art, for example, ethanol, isopropanol, ethyl carbonate, ethyl acetate, propylene glycol, 1,3-butanediol, dimethylformamide and oil, especially cottonseed oil, peanut oil, corn germ oil, olive oil, castor oil and sesame oil or a mixture of these substances.

In addition to these inert diluents, the composition may also comprise an adjuvant, such as wetting agent, emulsifying and suspending agents, sweetener, flavor and perfume.

In addition to the active compound, the suspension may comprise a suspending agent, for example, ethoxylated isostearyl alcohol, polyoxyethylene sorbitol and sorbitan esters, microcrystalline cellulose, aluminum methoxide and agar or a mixture of these substances.

A composition for parenteral injection may comprise a physiologically acceptable sterile aqueous or anhydrous solution, dispersion, suspension or emulsion, and a sterile powder for re-dissolving into a sterile injectable solution or dispersion. Suitable aqueous and nonaqueous carriers, diluents, solvents or excipients include water, ethanol, polyols and suitable mixtures thereof.

Dosage forms of the compounds of the present invention for topical administration include ointments, powders, patches, propellants and inhalants. The active ingredient is mixed with a physiologically acceptable carrier and any preservative, buffer, or propellant that may be required if necessary, under aseptic conditions.

The compounds of the present invention may be administered alone or in combination with other pharmaceutically acceptable compounds.

When a pharmaceutical composition is used, a safe and effective amount of a compound of the present invention is administrated to a mammal in need of the treatment, such as a human, wherein the dosage is a pharmaceutically effective administration dosage when administrated, and the daily administration dosage is usually 1-1000 mg, preferably 10-500 mg for an individual with a body weight of 60 kg. Of course, the specific dosage should also depend on the route of administration, the patient's health and other factors, which are all within the skills of a skilled physician.

The main advantages of the present invention include:

1. The compounds of the present invention have potent activities against Mycobacterium tuberculosis. The compounds of the present invention have excellent effects against multidrug-resistant Mycobacterium tuberculosis.

2. The compounds of the present invention have increased water solubility, and drug metabolism studies in animals have shown that the compounds of the present invention have excellent pharmacokinetic properties. This is important for the present compounds improve the anti Mycobacterium tuberculosis activity, improve efficacy, reduce side effects and save costs.

3. The compounds of the present invention have good safety to the cardiovascular system.

The various specific aspects, features and advantages of the above-mentioned compounds, methods and pharmaceutical compositions will be described in detail in the following description, and the contents of the present invention will become apparent. It is to be understood herein that the following detailed description and examples describe specific examples and are for reference only. After reading the description contents of the present invention, a person skilled in the art would be able to make various modifications or amendments to the present invention, and these equivalent forms likewise fall within the scope defined by the present application.

### DETAILED DESCRIPTION OF THE INVENTION

The present invention is explained more specifically in the following examples. It is to be understood, however, that these examples are intended to illustrate the invention and are not intended to limit the scope of the invention in any way. The experimental methods not specified for the specific conditions in the following examples are generally carried out in accordance with conventional conditions or in accordance with the conditions recommended by the manufacturer. Unless otherwise specified, the parts and percentages are parts by weight and percentages by weight.

In all the examples, the melting point was determined using an X-4 melting point apparatus and the thermometer was not corrected; ¹H-NMR was recorded with a Varian Mercury 300 or 400 nuclear magnetic resonance spectrometer and the chemical shift was expressed in δ (ppm); and MS was measured using an Shimadzu LC-MS-2020 mass spectrometer. When not specified, the silica gels for separation were all 200-300 mesh and the eluent ratios were all volume ratios.

### Example 1

### (S)-2-nitro-N-((6-(4-(4-(trifluoromethoxy)phenoxy)piperidin-1-yl)pyrid-3-yl)methyl)-6,7-dihydro-5H-imidazo[2,1-b][1,3]oxazin-6-amine (compound 1)

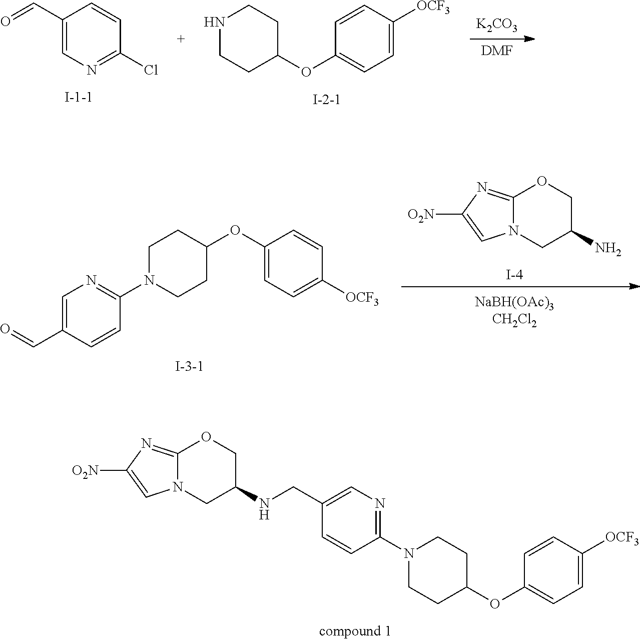

(1) 4-(4-(trifluoromethoxy)phenoxy)piperidine I-2-1 (200 mg, 0.77 mmol) (reference: U.S. Pat. No. 3,260,723) and 2-chloro-5-formylpyridine I-1-1 (130 mg, 0.92 mmol) were dissolved in DMF (5 mL), K₂CO₃ (317 mg, 2.30 mmol) was added to the solution dropwise and the mixture was reacted for 8 hours at 120° C. after the dropwise addition was completed. The reaction was completely cooled to room temperature, poured into ice water, extracted with ethyl acetate (20 mL*2), dried over anhydrous sodium sulfate, filtered, spin dried and purified by column chromatography (petroleum ether:ethyl acetate=4:1), giving intermediate I-3-1 (260 mg, yield: 93.2%) as a yellow oil.

Intermediate I-3-1: ¹H-NMR (400 MHz, CDCl₃) δ 9.78 (s, 1H), 8.57-8.53 (m, 1H), 7.93 (dd, J=9.1, 2.3 Hz, 1H), 7.18-7.12 (m, 2H), 6.95-6.88 (m, 2H), 6.70 (d, J=9.1 Hz, 1H), 4.62-4.55 (m, 1H), 4.02-3.92 (m, 2H), 3.81-3.72 (m, 2H), 2.08-1.98 (m, 2H), 1.95-1.83 (m, 2H).

(2) Intermediate I-3-1 (260 mg, 0.71 mmol) and triethylamine (93 mg, 0.92 mmol) were dissolved in dichloromethane (10 mL), then raw material I-4 (131 mg, 0.71 mmol) was added to the solution, the mixture was reacted at room temperature overnight, NaBH(OAc)₃ (602 mg, 2.84 mmol) was added thereto, and the reaction was continued at room temperature overnight. A solution of sodium bicarbonate (10 mL) was added, the layers were separated, the aqueous layer was extracted with dichloromethane (20 mL*2), the dichloromethane layers were combined, washed with saturated sodium chloride solution, dried over anhydrous sodium sulfate and spin dried, and the residue was purified by column chromatography (dichloromethane:methanol=50:1), giving compound 1 (205 mg, yield: 54.1%) as a pale yellow powder.

Compound 1: ¹H-NMR (400 MHz, CDCl₃) δ 8.08 (s, 1H), 7.45 (dd, J=8.7, 2.4 Hz, 1H), 7.37 (s, 1H), 7.14 (d, J=8.6 Hz, 2H), 6.94-6.87 (m, 2H), 6.68 (d, J=8.7 Hz, 1H), 4.73-4.50 (m, 1H), 4.44-4.31 (m, 2H), 4.15 (dd, J=12.4, 4.5 Hz, 1H), 3.90-3.79 (m, 3H), 3.84-3.74 (m, 2H), 3.42-3.37 (m, 3H), 2.09-1.98 (m, 2H), 1.88-1.80 (m, 2H). ESI-LR: 535.18 [M+1]⁺.

### Example 2

### (6S)-2-nitro-N-((6-(3-(4-(trifluoromethoxy)phenoxy)pyrrolidin-1-yl)pyrid-3-yl) methyl)-6,7-dihydro-5H-imidazo[2,1-b][1,3]oxazin-6-amine (compound 2)

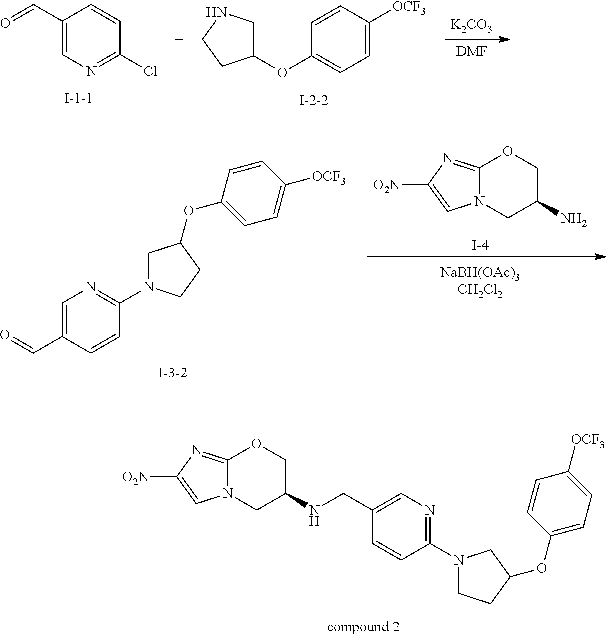

(1) 4-(4-(trifluoromethoxy)phenoxy)pyrrolidine I-2-2 (190 mg, 0.77 mmol) (reference: J. Med. Chem. 2012, 55(1), 312-326) and 2-chloro-5-formylpyridine I-1-1 (130 mg, 0.92 mmol) were used as raw materials, and the operation method was the same as the method of (1) in Example 1, giving intermediate I-3-2 (189 mg, yield: 69.7%).

Intermediate I-3-2: ¹H-NMR (400 MHz, CDCl₃) δ 9.75 (s, 1H), 8.57-8.53 (m, 1H), 7.93 (dd, J=9.1, 2.3 Hz, 1H), 7.18-7.12 (m, 2H), 6.95-6.88 (m, 2H), 6.70 (d, J=9.1 Hz, 1H), 4.64-4.57 (m, 1H), 4.22-4.17 (m, 2H), 3.57-3.50 (m, 2H), 2.08-1.98 (m, 1H), 1.95-1.90 (m, 1H).

(2) Intermediate I-3-2 (176 mg, 0.50 mmol) and I-4 (92 mg, 0.50 mmol) were used as raw materials, and the operation method was the same as the method of (2) in Example 1, giving pale yellow compound 2 (149 mg, yield: 57.3%).

Compound 2: ¹H-NMR (400 MHz, CDCl₃) δ 8.05 (s, 1H), 7.43 (dd, J=8.7, 2.4 Hz, 1H), 7.38 (s, 1H), 7.13 (d, J=8.6 Hz, 2H), 6.93-6.88 (m, 2H), 6.66 (d, J 8.7 Hz, 1H), 4.50-4.42 (m, 1H), 4.45-4.30 (m, 2H), 4.14-4.08 (m, 1H), 3.99-3.91 (m, 1H), 3.76-3.56 (m, 3H), 3.19 (d, J=0.4 Hz, 1H), 2.47 (s, 1H), 2.36-2.30 (m, 2H), 2.24-2.07 (m, 2H). ESI-LR: 521.46 [M+1]⁺.

### Example 3

### (6S)—N-((6-(3-fluoro-4-(4-(trifluoromethoxy)phenoxy)piperidin-1-yl)pyrid-3-yl) methyl)-2-nitro-6,7-dihydro-5H-imidazo[2,1-b][1,3]oxazin-6-amine (compound 3)

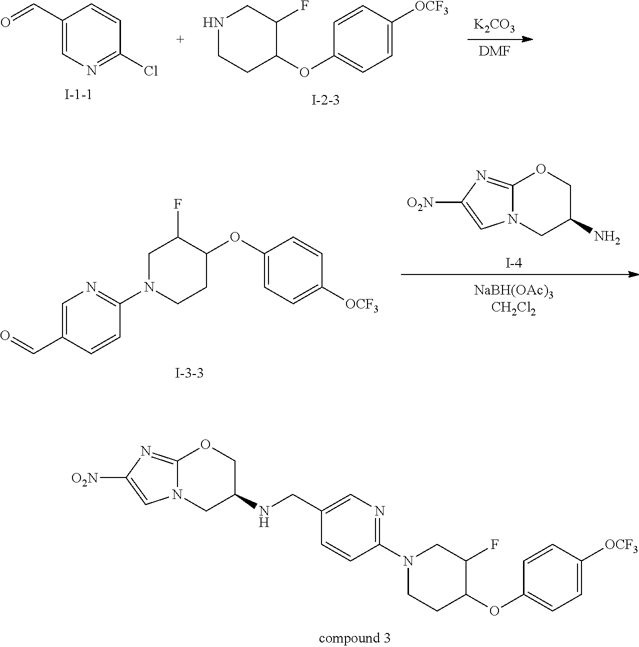

(1) 3-fluoro-4-(4-(trifluoromethoxy)phenoxy)piperidine I-2-3 (214 mg, 0.77 mmol) (reference: WO 2008124323) and 2-chloro-5-formylpyridine I-1-1 (130 mg, 0.92 mmol) were used as raw materials, and the operation method was the same as the method of (1) in Example 1, giving intermediate I-3-3 (242 mg, yield: 82.1%).

Intermediate I-3-3: ¹H-NMR (400 MHz, CDCl₃) δ 9.78 (s, 1H), 8.57-8.53 (m, 1H), 7.93 (dd, J=9.1, 2.3 Hz, 1H), 7.18-7.12 (m, 2H), 6.95-6.88 (m, 2H), 6.70 (d, J=9.1 Hz, 1H), 4.82-4.75 (m, 1H), 4.32-4.27 (m, 1H), 4.18-4.01 (m, 1H), 3.77-3.74 (m, 3H), 2.91-2.86 (m, 1H), 1.90-1.86 (m, 1H).

(2) Intermediate I-3-3 (230 mg, 0.60 mmol) and I-4 (110 mg, 0.60 mmol) were used as raw materials, and the operation method was the same as the method of (2) in Example 1, giving pale yellow compound 3 (180 mg, yield: 54.4%).

Compound 3: ¹H NMR (400 MHz, CDCl₃) δ 7.93 (d, J=2.3 Hz, 1H), 7.40 (dd, J=8.7, 2.4 Hz, 1H), 7.35 (s, 1H), 7.11 (d, J=8.6 Hz, 2H), 6.90-6.85 (m, 2H), 6.62 (d, J=8.7 Hz, 1H), 4.89-4.65 (m, 1H), 4.52-4.36 (m, 2H), 4.35-4.26 (m, 1H), 4.14-4.10 (m, 1H), 3.93-3.87 (m, 1H), 3.79-3.63 (m, 1H), 3.48 (dd, 1H), 3.40-3.23 (m, 2H), 3.19-3.03 (m, 1H), 2.25-2.13 (m, 2H), 1.98-1.84 (m, 2H). ESI-LR: 553.17 [M+1]⁺.

### Example 4

### (S)-2-nitro-N-((6-(4-(4-(trifluoromethoxy)phenyl)piperazin-1-yl)pyrid-3-yl)methyl)-6,7-dihydro-5H-imidazo[2,1-b][1,3]oxazin-6-amine (compound 4)

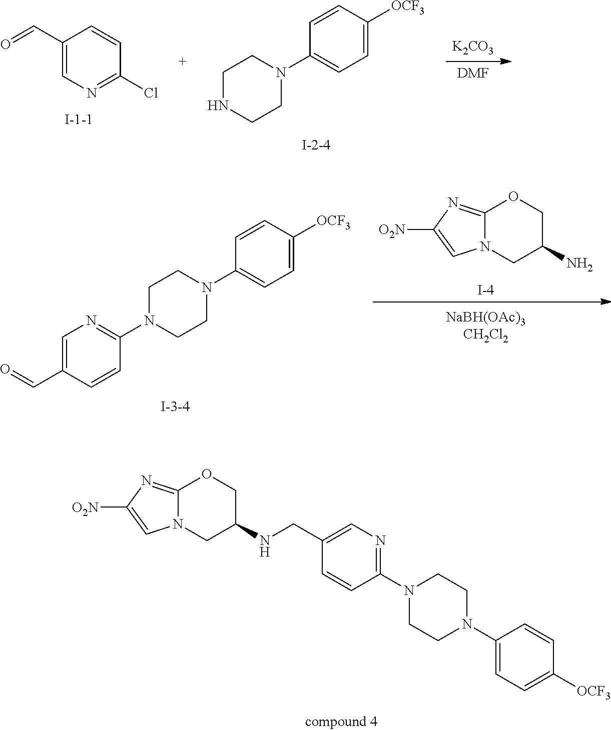

(1) 4-(4-(trifluoromethoxy)phenyl)piperazine I-2-4 (189 mg, 0.77 mmol) (reference: WO 2003105853) and 2-chloro-5-formylpyridine I-1-1 (130 mg, 0.92 mmol) were used as raw materials, and the operation method was the same as the method of (1) in Example 1, giving intermediate I-3-4 (242 mg, yield: 89.5%).

Intermediate I-3-4: ¹H-NMR (400 MHz, CDCl₃) δ 9.78 (s, 1H), 8.57-8.53 (m, 1H), 7.93 (dd, J=9.1, 2.3 Hz, 1H), 7.18-7.12 (m, 2H), 6.95-6.88 (m, 2H), 6.70 (d, J=9.1 Hz, 1H), 4.95-4.31 (m, 4H), 3.37-3.32 (m, 4H).

(2) Intermediate I-3-4 (211 mg, 0.60 mmol) and I-4 (110 mg, 0.60 mmol) were used as raw materials, and the operation method was the same as the method of (2) in Example 1, giving pale yellow compound 4 (205 mg, yield: 65.8%).

Compound 4: ¹H-NMR (400 MHz, CDCl₃) δ 8.11 (s, 1H), 7.48 (dd, J=8.6, 2.4 Hz, 1H), 7.36 (s, 1H), 7.13 (d, J=8.7 Hz, 2H), 6.94 (t, J=6.3 Hz, 2H), 6.69 (d, J=8.7 Hz, 1H), 4.41-4.35 (m, 2H), 4.14 (dd, J=12.3, 4.5 Hz, 1H), 3.92 (dd, J=12.2, 3.4 Hz, 1H), 3.86-3.76 (m, 2H), 3.79-3.70 (m, 4H), 3.40 (dd, J=4.7, 2.6 Hz, 1H), 3.31-3.25 (m, 4H). ESI-LR: 520.18 [M+1]⁺.

### Example 5

### (3S)—N-((6-(3-methyl-4-(4-(trifluoromethoxy)phenyl)piperazin-1-yl)pyrid-3-yl) methyl)-7-nitro-3,4-dihydro-2H-imidazo[2,1-b][1,3]oxazin-3-amine (compound 5)

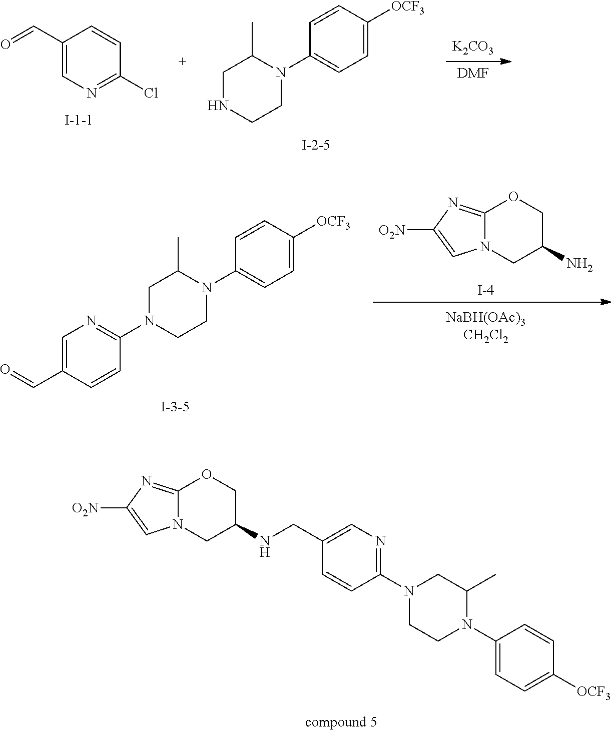

(1) 2-methyl-1-(4-(trifluoromethoxy)phenyl)piperazine I-2-5 (200 mg, 0.77 mmol) (reference: WO 2006079653) and 2-chloro-5-formylpyridine I-1-1 (130 mg, 0.92 mmol) were used as raw materials, and the operation method was the same as the method of (1) in Example 1, giving intermediate I-3-5 (240 mg, yield: 85.7%).

Intermediate I-3-5: ¹H-NMR (400 MHz, CDCl₃) δ 9.78 (s, 1H), 8.57-8.53 (m, 1H), 7.93 (dd, J=9.1, 2.3 Hz, 1H), 7.18-7.12 (m, 2H), 6.95-6.88 (m, 2H), 6.70 (d, J=9.1 Hz, 1H), 4.75-4.13 (m, 4H), 3.05-2.96 (m, 3H), 1.03 (d, J=6.5 Hz, 3H).

(2) Intermediate I-3-5 (219 mg, 0.60 mmol) and I-4 (110 mg, 0.60 mmol) were used as raw materials, and the operation method was the same as the method of (2) in Example 1, giving pale yellow compound 5 (191 mg, yield: 59.7%).

Compound 5: ¹H-NMR (400 MHz, CDCl₃) δ 8.13 (s, 1H), 7.52 (dd, J=8.6, 2.4 Hz, 1H), 7.38 (s, 1H), 7.14 (d, J=8.7 Hz, 2H), 6.97 (t, J=6.3 Hz, 2H), 6.71 (d, J=8.7 Hz, 1H), 4.44 (s, 1H), 4.40 (dd, J=8.6, 3.6 Hz, 2H), 4.3-4.25 (m, 1H), 4.18 (dd, J=12.4, 4.5 Hz, 1H), 3.99-3.92 (m, 1H), 3.90-3.84 (m, 1H), 3.75 (s, 2H), 3.60 (dd, J=12.9, 3.5 Hz, 1H), 3.46 (ddd, J=13.0, 6.6, 3.5 Hz, 1H), 3.40 (dd, J=4.4, 2.6 Hz, 1H), 3.28-3.21 (m, 1H), 3.20-3.11 (m, 1H), 1.01 (d, J=6.5 Hz, 3H). ESI-LR: 534.20 [M+1]⁺.

### Example 6

### (3S)—N-((6-(2-methyl-4-(4-(trifluoromethoxy)phenyl)piperazin-1-yl)pyrid-3-yl) methyl)-7-nitro-3,4-dihydro-2H-imidazo[2,1-b][1,3]oxazin-3-amine (compound 6)

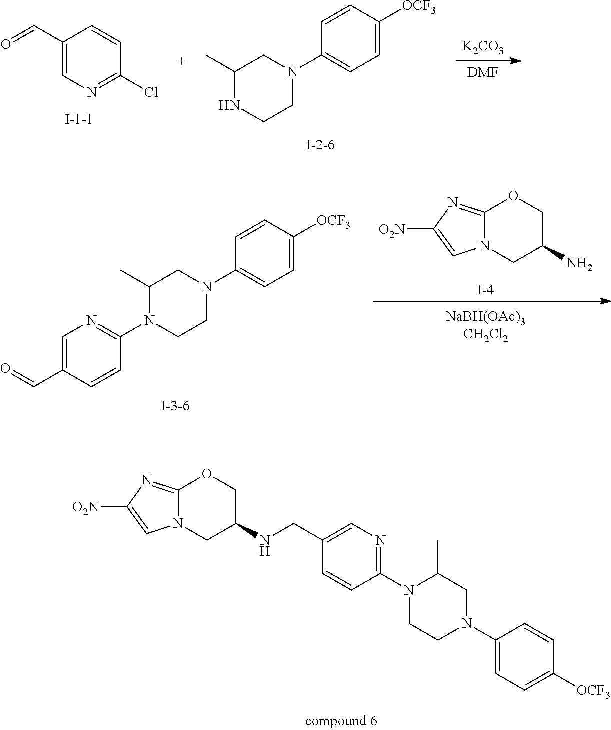

(1) 3-methyl-1-(4-(trifluoromethoxy)phenyl)piperazine I-2-6 (200 mg, 0.77 mmol) (reference: WO 2006079653) and 2-chloro-5-formylpyridine I-1-1 (130 mg, 0.92 mmol) were used as raw materials, and the operation method was the same as the method of (1) in Example 1, giving intermediate I-3-6 (191 mg, yield: 67.9%).

Intermediate I-3-6: ¹H-NMR (400 MHz, CDCl₃) δ 9.78 (s, 1H), 8.57-8.53 (m, 1H), 7.93 (dd, J=9.1, 2.3 Hz, 1H), 7.18-7.12 (m, 2H), 6.95-6.88 (m, 2H), 6.70 (d, J=9.1 Hz, 1H), 4.80-4.47 (m, 3H), 3.25-3.10 (m, 4H), 1.17 (d, J=6.5 Hz, 3H).

(2) Intermediate I-3-6 (183 mg, 0.50 mmol) and I-4 (92 mg, 0.50 mmol) were used as raw materials, and the operation method was the same as the method of (2) in Example 1, giving pale yellow compound 6 (169 mg, yield: 63.4%).

Compound 6: ¹H-NMR (400 MHz, CDCl₃) δ 8.13 (s, 1H), 7.52 (dd, J=8.6, 2.4 Hz, 1H), 7.38 (s, 1H), 7.14 (d, J=8.7 Hz, 2H), 6.97 (t, J=6.3 Hz, 2H), 6.71 (d, J=8.7 Hz, 1H), 4.89-4.82 (m, 1H), 4.40-4.30 (m, 1H), 4.16 (dd, J=12.8, 4.0 Hz, 1H), 3.97 (dd, J=12.7, 3.2 Hz, 1H), 3.70 (d, J=11.9 Hz, 1H), 3.61 (d, J=10.7 Hz, 3H), 3.29-3.20 (m, 3H), 2.94-2.90 (m, 1H), 2.78-2.64 (m, 2H), 1.20 (d, J=6.6 Hz, 3H). ESI-LR: 534.20 [M+1]⁺.

### Example 7

### (S)-7-nitro-N-((6-(4-(4-(trifluoromethoxy)phenyl)piperidin-1-yl)pyrid-3-yl)methyl)-3,4-dihydro-2H-imidazo[2,1-b][1,3]oxazin-3-amine (compound 7)

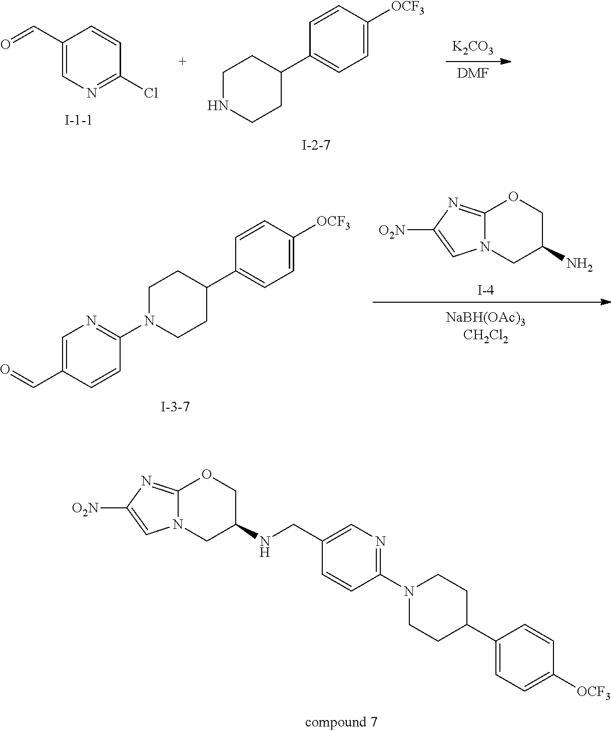

(1) 4-(4-(trifluoromethoxy)phenyl)piperidine I-2-7 (188 mg, 0.77 mmol) (reference: WO 2010081904) and 2-chloro-5-formylpyridine I-1-1 (130 mg, 0.92 mmol) were used as raw materials, and the operation method was the same as the method of (1) in Example 1, giving intermediate I-3-7 (248 mg, yield: 92.3%).

Intermediate I-3-7: ¹H-NMR (400 MHz, CDCl₃) δ 9.78 (s, 1H), 8.57-8.53 (m, 1H), 7.93 (dd, J=9.1, 2.3 Hz, 1H), 7.68-7.62 (m, 2H), 6.97-6.90 (m, 2H), 6.70 (d, J=9.1 Hz, 1H), 4.00-3.90 (m, 2H), 3.76-3.73 (m, 2H), 3.68-3.57 (m, 1H), 2.00-1.89 (m, 2H), 1.82-1.78 (m, 2H).

(2) Intermediate I-3-7 (210 mg, 0.60 mmol) and I-4 (110 mg, 0.60 mmol) were used as raw materials, and the operation method was the same as the method of (2) in Example 1, giving pale yellow compound 7 (167 mg, yield: 53.8%).

Compound 7: ¹H-NMR (400 MHz, CDCl₃) δ 8.13 (s, 1H), 7.53 (dd, J=8.6, 2.4 Hz, 1H), 7.39 (s, 1H), 7.15 (d, J=8.7 Hz, 2H), 6.97 (t, J=6.3 Hz, 2H), 6.73 (d, J=8.7 Hz, 1H), 4.79 (d, J=12.9 Hz, 2H), 4.41-4.29 (m, 2H), 4.13 (dd, J=12.7, 4.0 Hz, 1H), 3.98-3.91 (m, 1H), 3.61 (s, 2H), 2.97-2.81 (m, 4H), 1.85-1.81 (m, 2H), 1.52-1.45 (m, 2H). ESI-LR: 519.19 [M+1]⁺.

### Example 8

### (S)-1-(5-(((7-nitro-3,4-dihydro-2H-imidazo[2,1-b][1,3]oxazin-3-yl)amino)methy 1)pyridin-2-yl)-4-(4-(trifluoromethoxy)phenyl)piperidin-4-ol (compound 8)

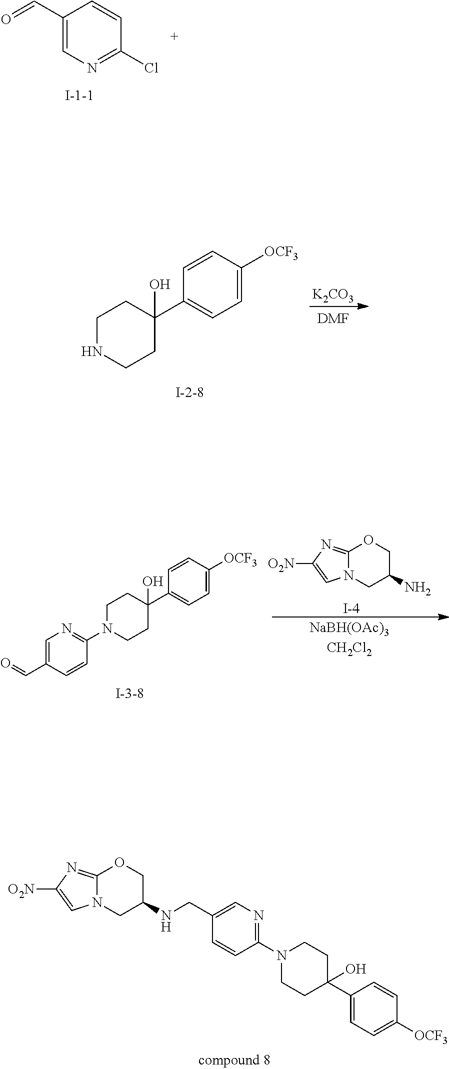

(1) 4-(4-(trifluoromethoxy)phenyl)piperidin-4-ol I-2-8 (200 mg, 0.77 mmol) (reference: WO 2005118587) and 2-chloro-5-formylpyridine I-1-1 (130 mg, 0.92 mmol) were used as raw materials, and the operation method was the same as the method of (1) in Example 1, giving intermediate I-3-8 (214 mg, yield: 75.9%).

Intermediate I-3-8: ¹H-NMR (400 MHz, CDCl₃) δ 9.78 (s, 1H), 8.57-8.53 (m, 1H), 7.93 (dd, J=9.1, 2.3 Hz, 1H), 7.24-7.18 (m, 2H), 6.96-6.89 (m, 2H), 6.70 (d, J=9.1 Hz, 1H), 4.00-3.90 (m, 2H), 3.76-3.73 (m, 2H), 2.14-2.03 (m, 2H), 1.96-1.91 (m, 2H).

(2) Intermediate I-3-8 (183 mg, 0.50 mmol) and I-4 (92 mg, 0.50 mmol) were used as raw materials, and the operation method was the same as the method of (2) in Example 1, giving pale yellow compound 8 (89 mg, yield: 33.6%).

Compound 8: ¹H-NMR (400 MHz, CDCl₃) δ 8.15 (s, 1H), 7.54 (dd, J=8.6, 2.4 Hz, 1H), 7.39 (s, 1H), 7.16 (d, J=8.7 Hz, 2H), 6.97 (t, J=6.3 Hz, 2H), 6.73 (d, J=8.7 Hz, 1H), 4.34 (dt, J=11.2, 8.0 Hz, 2H), 4.13-4.09 (m, 1H), 3.98-3.79 (m, 3H), 3.59 (d, J=11.6 Hz, 2H), 3.38 (s, 1H), 3.26 (t, J=12.6 Hz, 2H), 2.23-2.19 (m, 2H), 1.88-1.84 (m, 2H). ESI-LR: 535.18 [M+1]⁺.

### Example 9

### (S)—N-((6-(4-methoxy-4-(4-(trifluoromethoxy)phenyl)piperidin-1-yl)pyrid-3-yl) methyl)-7-nitro-3,4-dihydro-2H-imidazo[2,1-b][1,3]oxazin-3-amine (compound 9)

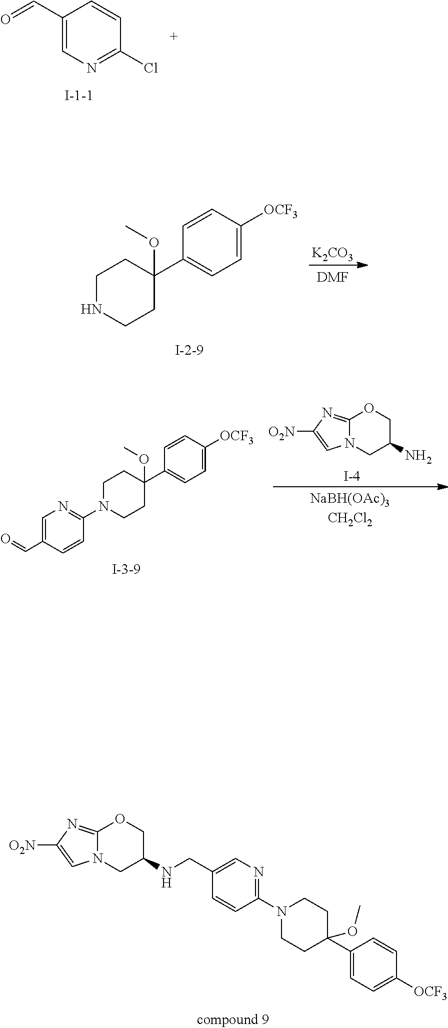

(1) 4-methoxy-4-(4-(trifluoromethoxy)phenyl)piperidine I-2-9 (212 mg, 0.77 mmol) (reference: WO 2013096744) and 2-chloro-5-formylpyridine I-1-1 (130 mg, 0.92 mmol) were used as raw materials, and the operation method was the same as the method of (1) in Example 1, giving intermediate I-3-9 (228 mg, yield: 77.9%).

Intermediate I-3-9: ¹H-NMR (400 MHz, CDCl₃) δ 9.78 (s, 1H), 8.57-8.53 (m, 1H), 7.93 (dd, J=9.1, 2.3 Hz, 1H), 7.24-7.18 (m, 2H), 6.96-6.89 (m, 2H), 6.70 (d, J=9.1 Hz, 1H), 4.00-3.90 (m, 2H), 3.76-3.73 (m, 2H), 3.57 (s, 3H), 2.12-2.01 (m, 2H), 1.94-1.89 (m, 2H).

(2) Intermediate I-3-9 (190 mg, 0.50 mmol) and I-4 (92 mg, 0.50 mmol) were used as raw materials, and the operation method was the same as the method of (2) in Example 1, giving pale yellow compound 9 (133 mg, yield: 47.6%).

Compound 9: ¹H-NMR (400 MHz, CDCl₃) δ 8.14 (s, 1H), 7.53 (dd, J=8.6, 2.4 Hz, 1H), 7.39 (s, 1H), 7.14 (d, J=8.7 Hz, 2H), 6.97 (t, J=6.3 Hz, 2H), 6.73 (d, J=8.7 Hz, 1H), 4.40 (dt, J=11.2, 8.0 Hz, 2H), 4.11-4.07 (m, 1H), 4.00-3.81 (m, 6H), 3.59 (d, J=11.6 Hz, 2H), 3.38 (s, 1H), 3.26-3.20 (m, 2H), 2.23-2.19 (m, 2H), 1.88-1.84 (m, 2H). ESI-LR: 549.20 [M+1]⁺.

### Example 10

### (S)-1-(5-(((7-nitro-3,4-dihydro-2H-imidazo[2,1-b][1,3]oxazin-3-yl)amino)methyl)pyridin-2-yl)-4-(4-(trifluoromethoxy)phenyl)piperidine-4-carbonitrile (compound 10)

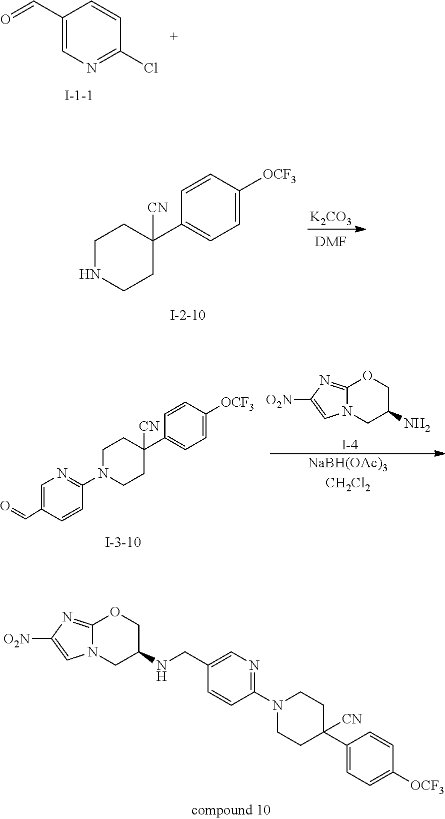

(1) 4-(4-(trifluoromethoxy)phenyl)piperidine-4-carbonitrile I-2-10 (208 mg, 0.77 mmol) (reference: J. Med. Chem. 2011, 54(13), 4773-4780) and 2-chloro-5-formylpyridine I-1-1 (130 mg, 0.92 mmol) were used as raw materials, and the operation method was the same as the method of (1) in Example 1, giving intermediate I-3-10 (234 mg, yield: 81.3%).

Intermediate I-3-10: ¹H-NMR (400 MHz, CDCl₃) δ 9.78 (s, 1H), 8.57-8.53 (m, 1H), 7.93 (dd, J=9.1, 2.3 Hz, 1H), 7.08-7.02 (m, 2H), 6.94-6.87 (m, 2H), 6.70 (d, J=9.1 Hz, 1H), 4.03-3.91 (m, 2H), 3.77-3.74 (m, 2H), 2.32-2.23 (m, 2H), 2.14-2.09 (m, 2H).

(2) Intermediate I-3-10 (225 mg, 0.60 mmol) and I-4 (110 mg, 0.60 mmol) were used as raw materials, and the operation method was the same as the method of (2) in Example 1, giving pale yellow compound 10 (158 mg, yield: 48.6%).

Compound 10: ¹H-NMR (400 MHz, CDCl₃) δ 8.14 (s, 1H), 7.53 (dd, J=8.6, 2.4 Hz, 1H), 7.39 (s, 1H), 7.14 (d, J=8.7 Hz, 2H), 6.97 (t, J=6.3 Hz, 2H), 6.73 (d, J=8.7 Hz, 1H), 4.43 (dt, J=11.2, 8.0 Hz, 2H), 4.13-4.08 (m, 1H), 4.03-3.92 (m, 3H), 3.61 (d, J=11.6 Hz, 2H), 3.42 (s, 1H), 3.32-3.25 (m, 2H), 2.94-2.87 (m, 2H), 2.30-2.25 (m, 2H). ESI-LR: 543.19 [M+1]⁺.

### Example 11

### (6S)-2-nitro-N-((6-(5-(4-(trifluoromethoxy)phenyl)hexahydropyrrolo[3,4-c]pyrrol-2(1H)-yl)pyrid-3-yl)methyl)-6,7-dihydro-5H-imidazo[2,1-b][1,3]oxazin-6-amine (compound 11)

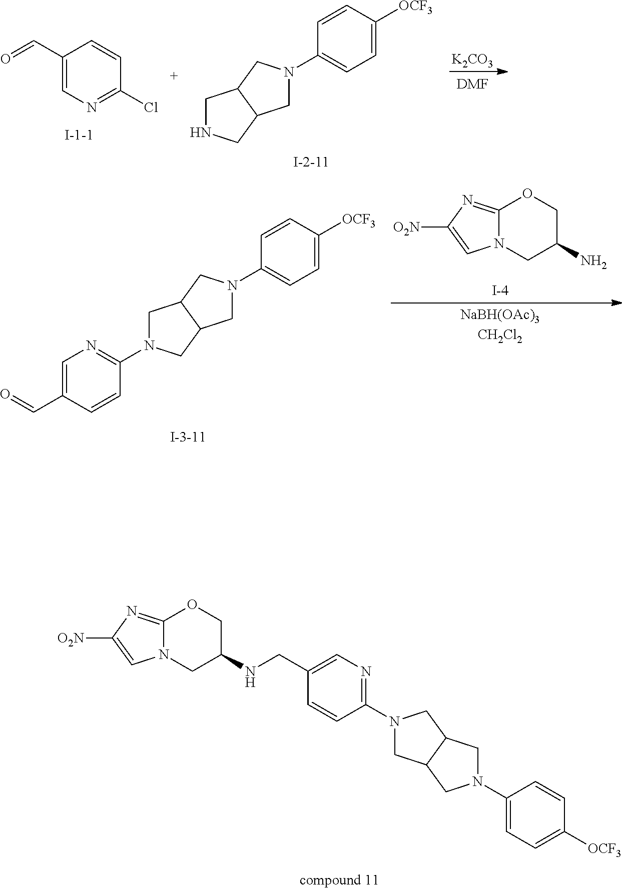

(1) 2-(4-(trifluoromethoxy)phenyl)octahydropyrrolo[3,4]pyrrole I-2-11 (209 mg, 0.77 mmol) (reference: WO 2013021054) and 2-chloro-5-formylpyridine I-1-1 (130 mg, 0.92 mmol) were used as raw materials, and the operation method was the same as the method of (1) in Example 1, giving intermediate I-3-11 (250 mg, yield: 86.2%).

Intermediate I-3-11: ¹H-NMR (400 MHz, CDCl₃) δ 9.78 (s, 1H), 8.57-8.53 (m, 1H), 7.93 (dd, J=9.1, 2.3 Hz, 1H), 7.18-7.12 (m, 2H), 6.95-6.88 (m, 2H), 6.70 (d, J=9.1 Hz, 1H), 3.83-3.71 (m, 4H), 3.49-3.35 (m, 4H), 3.18 (s, 2H).

(2) Intermediate I-3-11 (226 mg, 0.60 mmol) and I-4 (110 mg, 0.60 mmol) were used as raw materials, and the operation method was the same as the method of (2) in Example 1, giving pale yellow compound 11 (177 mg, yield: 54.1%).

Compound 11: ¹H-NMR (400 MHz, CDCl₃) δ 8.04 (d, J=2.0 Hz, 1H), 7.42 (dd, J=8.7, 2.3 Hz, 1H), 7.35 (s, 1H), 7.08 (d, J=8.3 Hz, 2H), 6.49 (d, J=9.1 Hz, 2H), 6.35 (d, J=8.4 Hz, 1H), 4.41-4.32 (m, 2H), 4.12 (dd, J=12.3, 4.5 Hz, 1H), 3.90 (dd, J=12.4, 3.4 Hz, 1H), 3.83-3.71 (m, 4H), 3.63-3.55 (m, 2H), 3.49-3.35 (m, 4H), 3.27 (dd, J=9.5, 3.8 Hz, 2H), 3.18 (s, 2H). ESI-LR: 546.20 [M+1]⁺.

### Example 12

### (6S)-2-nitro-N-((6-(5-(4-(trifluoromethoxy)phenyl)-2,5-diazabicyclo[2.2.1]heptan-2-yl)pyrid-3-yl)methyl)-6,7-dihydro-5H-imidazo[2,1-b][1,3]oxazin-6-amine (compound 12)

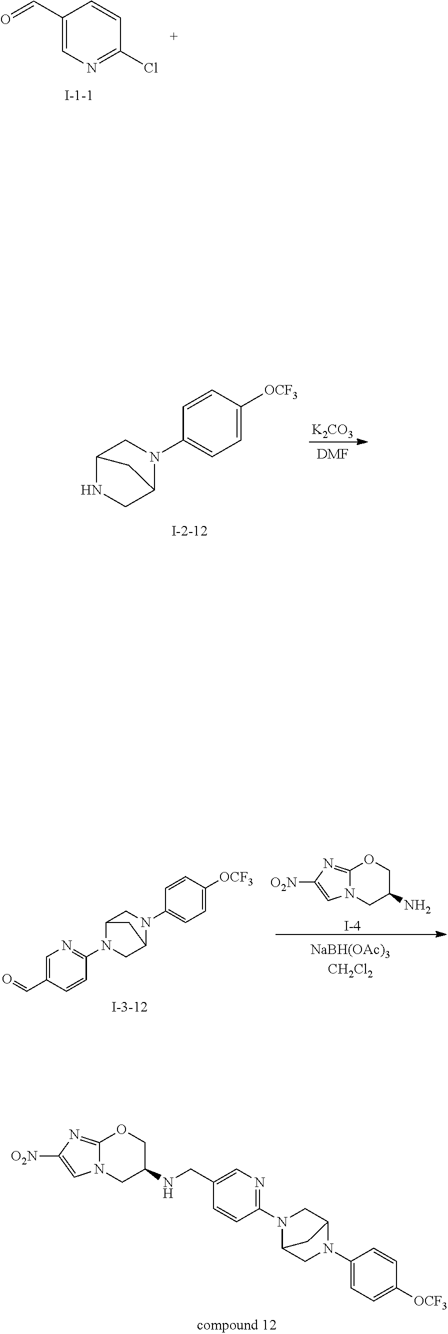

(1) 2-(4-(trifluoromethoxy)phenyl)-2,5-diazabicyclo[2.2.1]heptane I-2-12 (198 mg, 0.77 mmol) (reference: WO 2005117909) and 2-chloro-5-formylpyridine I-1-1 (130 mg, 0.92 mmol) were used as raw materials, and the operation method was the same as the method of (1) in Example 1, giving intermediate I-3-12 (210 mg, yield: 75.3%).

Intermediate I-3-12: ¹H-NMR (400 MHz, CDCl₃) δ 9.78 (s, 1H), 8.57-8.54 (m, 1H), 7.93 (dd, J=9.1, 2.3 Hz, 1H), 7.18-7.11 (m, 2H), 6.95-6.89 (m, 2H), 6.70 (d, J=9.1 Hz, 1H), 3.71-3.65 (m, 3H), 3.31-3.25 (m, 3H), 1.78-1.73 (m, 1H), 1.53-1.47 (m, 1H).

(2) Intermediate I-3-12 (181 mg, 0.50 mmol) and I-4 (84 mg, 0.50 mmol) were used as raw materials, and the operation method was the same as the method of (2) in Example 1, giving pale yellow compound 12 (152 mg, yield: 57.6%).

Compound 12: ¹H-NMR (400 MHz, CDCl₃) δ 8.11 (s, 1H), 7.48 (dd, J=8.6, 2.4 Hz, 1H), 7.36 (s, 1H), 7.13 (d, J=8.7 Hz, 2H), 6.94 (t, J=6.3 Hz, 2H), 6.69 (d, J=8.7 Hz, 1H), 4.40-4.38 (m, 1H), 4.32 (dd, J=12.0, 4.3 Hz, 1H), 4.13 (dd, J=12.3, 4.5 Hz, 1H), 3.90 (dd, J=12.2, 3.4 Hz, 1H), 3.86-3.76 (m, 2H), 3.70-3.63 (m, 3H), 3.40 (dd, J=4.7, 2.6 Hz, 1H), 3.30-3.24 (m, 3H), 1.77-1.72 (m, 1H), 1.52-1.49 (m, 1H). ESI-LR: 532.18 [M+1]⁺.

### Example 13

### (S)-2-nitro-N-((6-(2-(4-(trifluoromethoxy)phenyl)-2,7-diazaspiro[3.5]nonan-7-yl)pyrid-3-yl)methyl)-6,7-dihydro-5H-imidazo[2,1-b][1,3]oxazin-6-amine (compound 13)

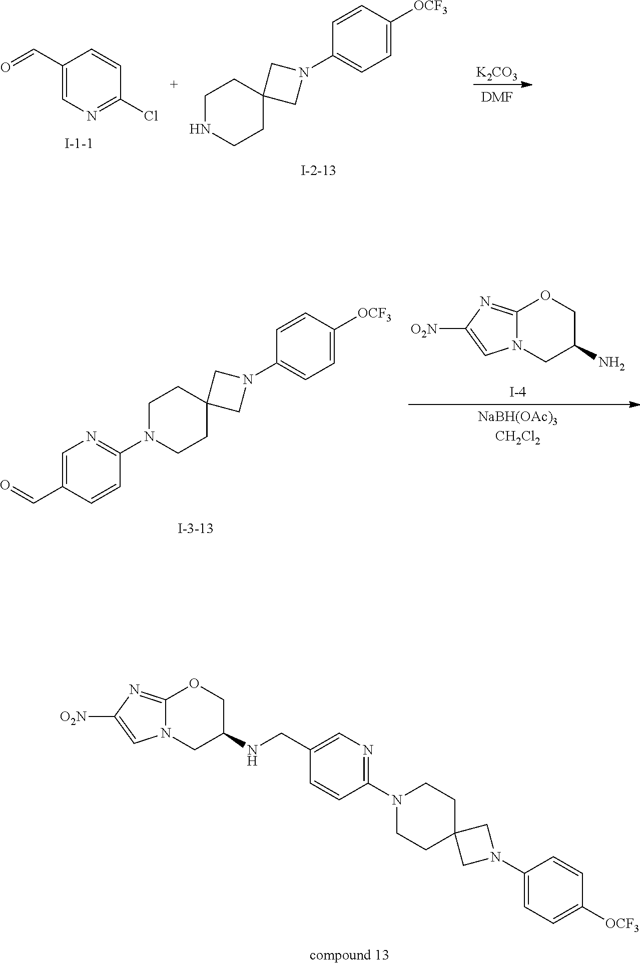

(1) 2-(4-(trifluoromethoxy)phenyl)-2,7-diazaspiro[3.5]nonane I-2-13 (220 mg, 0.77 mmol) (reference: WO 2010108268) and 2-chloro-5-formylpyridine I-1-1 (130 mg, 0.92 mmol) were used as raw materials, and the operation method was the same as the method of (1) in Example 1, giving intermediate I-3-13 (231 mg, yield: 76.8%).

Intermediate I-3-13: ¹H-NMR (400 MHz, CDCl₃) δ 9.78 (s, 1H), 8.57-8.53 (m, 1H), 7.93 (dd, J=9.1, 2.3 Hz, 1H), 7.18-7.12 (m, 2H), 6.95-6.88 (m, 2H), 6.70 (d, J=9.1 Hz, 1H), 5.21-4.61 (m, 4H), 3.57-3.50 (m, 4H), 1.59-1.51 (m, 4H).

(2) Intermediate I-3-13 (195 mg, 0.50 mmol) and I-4 (84 mg, 0.50 mmol) were used as raw materials, and the operation method was the same as the method of (2) in Example 1, giving pale yellow compound 13 (119 mg, yield: 42.8%).

Compound 13: ¹H-NMR (400 MHz, CDCl₃) δ 8.14 (s, 1H), 7.53 (dd, J=8.6, 2.4 Hz, 1H), 7.39 (s, 1H), 7.14 (d, J=8.7 Hz, 2H), 6.97 (t, J=6.3 Hz, 2H), 6.73 (d, J=8.7 Hz, 1H), 4.40 (dt, J=11.2, 8.0 Hz, 2H), 4.11-4.07 (m, 1H), 4.00-3.81 (m, 6H), 3.59-3.50 (m, 6H), 3.39 (s, 1H), 3.28-3.21 (m, 2H), 2.27-2.20 (m, 2H), 1.95-1.89 (m, 2H). ESI-LR: 560.22 [M+1]⁺.

### Example 14

### (6S)-2-nitro-N-((6-(3-(4-(trifluoromethoxy)phenoxy)-8-azabicyclo[3.2.1]octan-8-yl)pyrid-3-yl)methyl)-6,7-dihydro-5H-imidazo[2,1-b][1,3]oxazin-6-amine (compound 14)

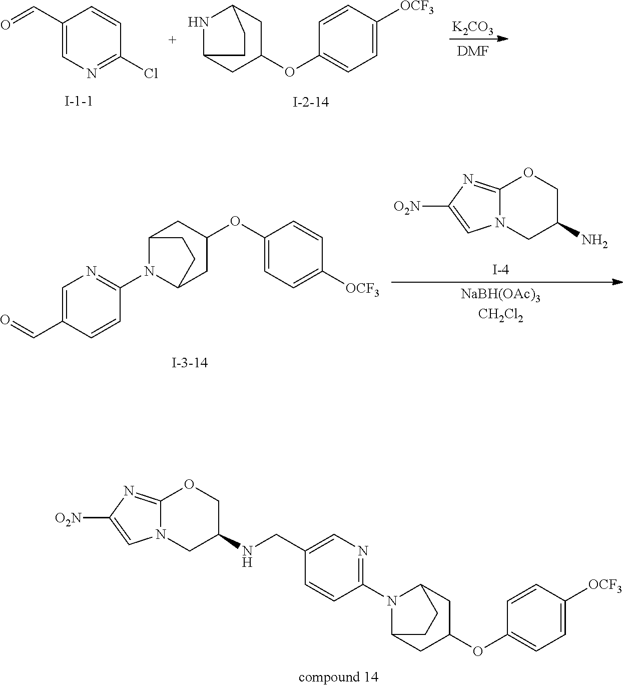

(1) 2-(4-(trifluoromethoxy)phenyl)-8-azabicyclo[3.2.1]octane I-2-14 (220 mg, 0.77 mmol) (reference: WO 2007079239) and 2-chloro-5-formylpyridine I-1-1 (130 mg, 0.92 mmol) were used as raw materials, and the operation method was the same as the method of (1) in Example 1, giving intermediate I-3-14 (219 mg, yield: 72.8%).

Intermediate I-3-14: ¹H-NMR (400 MHz, CDCl₃) δ 9.78 (s, 1H), 8.57-8.53 (m, 1H), 7.93 (dd, J=9.1, 2.3 Hz, 1H), 7.18-7.12 (m, 2H), 6.95-6.88 (m, 2H), 6.70 (d, J=9.1 Hz, 1H), 4.62-4.54 (m, 1H), 3.57-3.51 (m, 2H), 2.05-1.95 (m, 2H), 1.87-1.83 (m, 2H), 1.79-1.75 (m, 2H), 1.47-1.50 (m, 2H).

(2) Intermediate I-3-14 (196 mg, 0.50 mmol) and I-4 (84 mg, 0.50 mmol) were used as raw materials, and the operation method was the same as the method of (2) in Example 1, giving pale yellow compound 14 (141 mg, yield: 50.4%).

Compound 14: ¹H-NMR (400 MHz, CDCl₃) δ 8.11 (s, 1H), 7.45 (dd, J=8.7, 2.4 Hz, 1H), 7.36 (s, 1H), 7.15 (d, J=8.6 Hz, 2H), 6.94-6.89 (m, 2H), 6.67 (d, J 8.7 Hz, 1H), 4.73-4.50 (m, 1H), 4.42-4.30 (m, 2H), 4.13 (dd, J=12.4, 4.5 Hz, 1H), 3.87-3.79 (m, 3H), 3.81-3.72 (m, 2H), 3.42-3.37 (m, 1H), 2.07-1.98 (m, 2H), 1.88-1.80 (m, 2H), 1.70-1.65 (m, 2H), 1.45-1.48 (m, 2H). ESI-LR: 561.20 [M+1]⁺.

### Example 15

### (S)-2-nitro-N-((6-(4-(4-(trifluoromethoxy)phenoxy)piperidin-1-yl)pyrimidin-3-yl)methyl)-6,7-dihydro-5H-imidazo[2,1-b][1,3]oxazin-6-amine (compound 15)

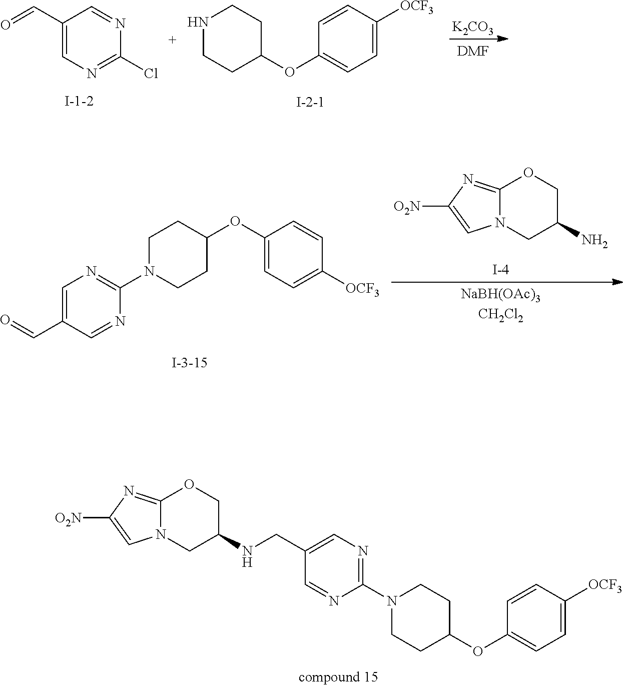

(1) 4-(4-(trifluoromethoxy)phenoxy)piperidine I-2-1 (200 mg, 0.77 mmol) and 2-chloro-5-formylpyrimidine I-1-2 (130 mg, 0.92 mmol) were used as raw materials, and the operation method was the same as the method of (1) in Example 1, giving intermediate I-3-15 (230 mg, yield: 81.7%).

Intermediate I-3-15: ¹H-NMR (400 MHz, CDCl₃) δ 9.79 (s, 1H), 8.75 (s, 2H), 7.18-7.14 (m, 2H), 6.95-6.92 (m, 2H), 4.62-4.54 (m, 1H), 4.02-3.92 (m, 2H), 3.57-3.51 (m, 2H), 2.05-1.95 (m, 2H), 1.87-1.83 (m, 2H).

(2) Intermediate I-3-15 (220 mg, 0.60 mmol) and I-4 (110 mg, 0.60 mmol) were used as raw materials, and the operation method was the same as the method of (2) in Example 1, giving pale yellow compound 15 (186 mg, yield: 58.1%).

Compound 15: ¹H-NMR (400 MHz, CDCl₃) δ 8.30 (s, 2H), 8.03 (s, 1H), 7.28 (d, J=8.7 Hz, 2H), 7.09 (d, J=9.1 Hz, 2H), 4.72-4.62 (m, 1H), 4.45-4.33 (m, 2H), 4.23-4.11 (m, 3H), 4.00-3.92 (m, 1H), 3.61 (s, 2H), 3.54-3.44 (m, 2H), 3.27-3.19 (m, 1H), 2.01-1.92 (m, 2H), 1.61-1.49 (m, 2H). ESI-LR: 536.18 [M+1]⁺.

### Example 16

### (6S)-2-nitro-N-((6-(3-(4-(trifluoromethoxy)phenoxy)pyrrolidin-1-yl)pyrimidin-3-yl)methyl)-6,7-dihydro-5H-imidazo[2,1-b][1,3]oxazin-6-amine (compound 16)

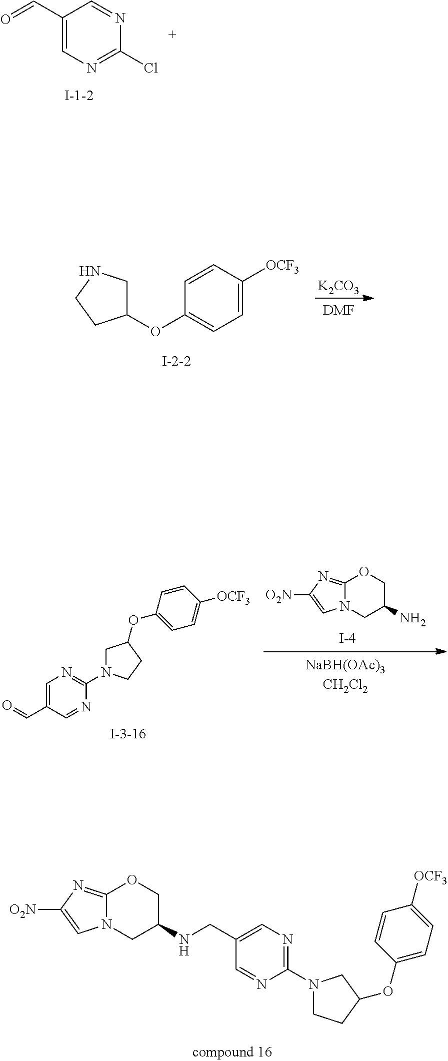

(1) 4-(4-(trifluoromethoxy)phenoxy)pyrrolidine I-2-2 (190 mg, 0.77 mmol) and 2-chloro-5-formylpyrimidine 1-1-2 (130 mg, 0.92 mmol) were used as raw materials, and the operation method was the same as the method of (1) in Example 1, giving intermediate I-3-16 (183 mg, yield: 67.3%).

Intermediate I-3-16: ¹H-NMR (400 MHz, CDCl₃) δ 9.79 (s, 1H), 8.75 (s, 2H), 7.18-7.14 (m, 2H), 6.95-6.92 (m, 2H), 4.64-4.57 (m, 1H), 4.22-4.17 (m, 2H), 3.57-3.50 (m, 2H), 2.08-1.98 (m, 1H), 1.95-1.90 (m, 1H).

(2) Intermediate I-3-16 (176 mg, 0.50 mmol) and I-4 (92 mg, 0.50 mmol) were used as raw materials, and the operation method was the same as the method of (2) in Example 1, giving pale yellow compound 16 (156 mg, yield: 60.1%).

Compound 16: ¹H-NMR (400 MHz, CDCl₃) δ 8.32 (s, 2H), 8.03 (s, 1H), 7.21 (d, J=8.7 Hz, 2H), 7.05 (d, J=9.2 Hz, 2H), 4.50-4.42 (m, 1H), 4.45-4.30 (m, 2H), 4.14-4.08 (m, 1H), 3.99-3.91 (m, 1H), 3.76-3.56 (m, 3H), 3.19 (d, J=0.4 Hz, 1H), 2.47 (s, 1H), 2.36-2.30 (m, 2H), 2.24-2.07 (m, 2H). ESI-LR: 522.16 [M+1]⁺.

### Example 17

### (6S)—N-((6-(3-fluoro-4-(4-(trifluoromethoxy)phenoxy)piperidin-1-yl)pyrimidin-3-yl)methyl)-2-nitro-6,7-dihydro-5H-imidazo[2,1-b][1,3]oxazin-6-amine (compound 17)

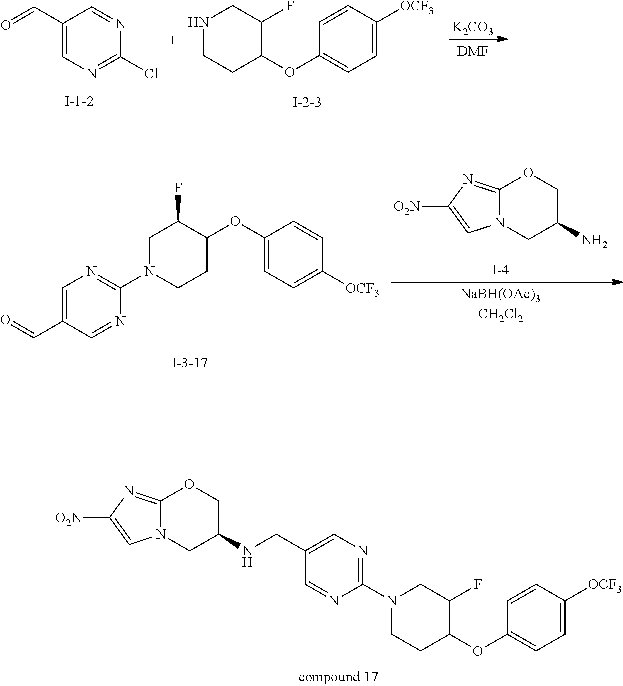

(1) 3-fluoro-4-(4-(trifluoromethoxy)phenoxy)piperidine I-2-3 (214 mg, 0.77 mmol) and 2-chloro-5-formylpyrimidine I-1-2 (130 mg, 0.92 mmol) were used as raw materials, and the operation method was the same as the method of (1) in Example 1, giving intermediate I-3-17 (233 mg, yield: 78.5%).

Intermediate I-3-17: ¹H-NMR (400 MHz, CDCl₃) δ 9.79 (s, 1H), 8.75 (s, 2H), 7.18-7.14 (m, 2H), 6.95-6.92 (m, 2H), 4.82-4.75 (m, 1H), 4.32-4.27 (m, 1H), 4.18-4.01 (m, 1H), 3.77-3.74 (m, 3H), 2.91-2.86 (m, 1H), 1.90-1.86 (m, 1H).

(2) Intermediate I-3-17 (230 mg, 0.60 mmol) and I-4 (110 mg, 0.60 mmol) were used as raw materials, and the operation method was the same as the method of (2) in Example 1, giving pale yellow compound 17 (153 mg, yield: 46.2%).

Compound 17: ¹H NMR (400 MHz, CDCl₃) δ 8.34 (s, 2H), 8.03 (s, 1H), 7.21 (d, J=8.7 Hz, 2H), 7.05 (d, J=9.2 Hz, 2H), 4.89-4.65 (m, 1H), 4.52-4.36 (m, 2H), 4.35-4.26 (m, 1H), 4.14-4.10 (m, 1H), 3.93-3.87 (m, 1H), 3.79-3.63 (m, 1H), 3.48 (dd, 1H), 3.40-3.23 (m, 2H), 3.19-3.03 (m, 1H), 2.25-2.13 (m, 2H), 1.98-1.84 (m, 2H). ESI-LR: 554.18 [M+1]⁺.

### Example 18

### (S)-2-nitro-N-((6-(4-(4-(trifluoromethoxy)phenyl)piperazin-1-yl)pyrimidin-3-yl) methyl)-6,7-dihydro-5H-imidazo[2,1-b][1,3]oxazin-6-amine (compound 18)

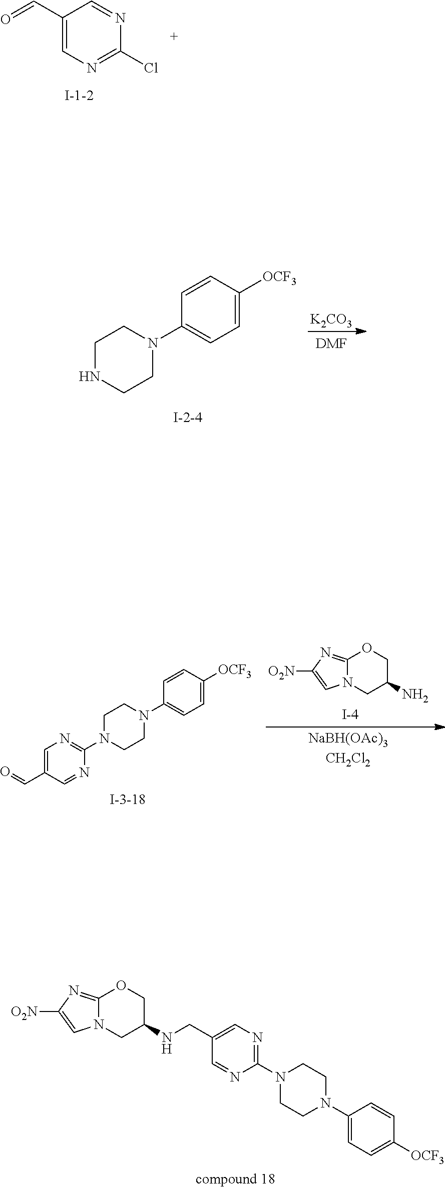

(1) 4-(4-(trifluoromethoxy)phenyl)piperazine I-2-4 (189 mg, 0.77 mmol) and 2-chloro-5-formylpyrimidine I-1-2 (130 mg, 0.92 mmol) were used as raw materials, and the operation method was the same as the method of (1) in Example 1, giving intermediate I-3-18 (232 mg, yield: 85.7%).

Intermediate I-3-18: ¹H-NMR (400 MHz, CDCl₃) δ 9.79 (s, 1H), 8.75 (s, 2H), 7.18-7.14 (m, 2H), 6.95-6.92 (m, 2H), 4.16-4.13 (m, 4H), 3.27-3.24 (m, 4H).

(2) Intermediate I-3-18 (211 mg, 0.60 mmol) and I-4 (110 mg, 0.60 mmol) were used as raw materials, and the operation method was the same as the method of (2) in Example 1, giving pale yellow compound 18 (180 mg, yield: 57.9%).

Compound 18: ¹H-NMR (400 MHz, CDCl₃) δ 8.33 (s, 2H), 8.03 (s, 1H), 7.21 (d, J=8.7 Hz, 2H), 7.05 (d, J=9.2 Hz, 2H), 4.41-4.35 (m, 2H), 4.14 (dd, J=12.3, 4.5 Hz, 1H), 3.92 (dd, J=12.2, 3.4 Hz, 1H), 3.82-3.70 (m, 4H), 3.62 (s, 2H), 3.31-3.21 (m, 5H). ESI-LR: 521.18 [M+1]⁺.

### Example 19

### (3S)—N-((6-(3-methyl-4-(4-(trifluoromethoxy)phenyl)piperazin-1-yl)pyrimidin-3-yl)methyl)-7-nitro-3,4-dihydro-2H-imidazo[2,1-b][1,3]oxazin-3-amine (compound 19)

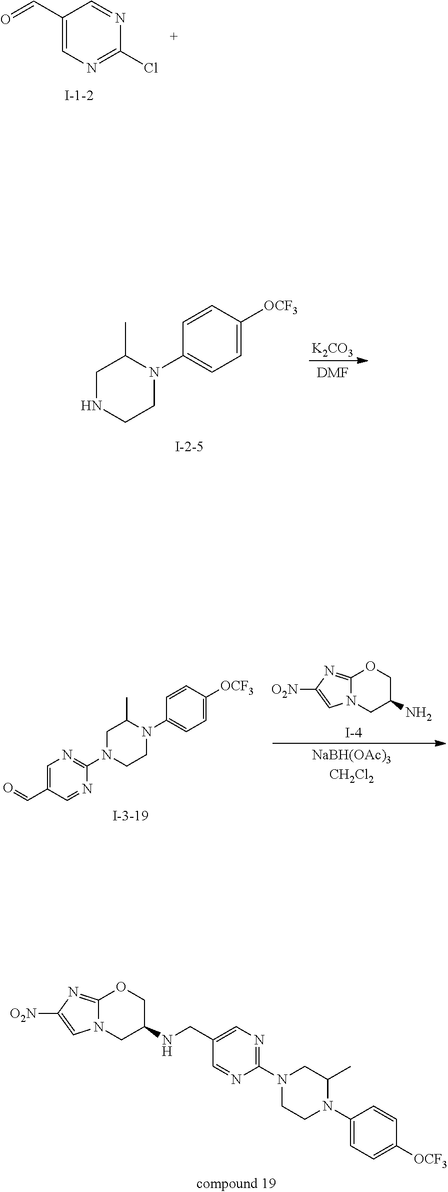

(1) 2-methyl-1-(4-(trifluoromethoxy)phenyl)piperazine I-2-5 (200 mg, 0.77 mmol) and 2-chloro-5-formylpyrimidine I-1-2 (130 mg, 0.92 mmol) were used as raw materials, and the operation method was the same as the method of (1) in Example 1, giving intermediate I-3-19 (238 mg, yield: 84.6%).

Intermediate I-3-19: ¹H-NMR (400 MHz, CDCl₃) δ 9.79 (s, 1H), 8.75 (s, 2H), 7.18-7.14 (m, 2H), 6.95-6.92 (m, 2H), 4.75-4.13 (m, 4H), 3.05-2.96 (m, 3H), 1.03 (d, J=6.5 Hz, 3H).

(2) Intermediate I-3-19 (219 mg, 0.60 mmol) and I-4 (110 mg, 0.60 mmol) were used as raw materials, and the operation method was the same as the method of (2) in Example 1, giving pale yellow compound 19 (162 mg, yield: 50.8%).

Compound 19: ¹H-NMR (400 MHz, CDCl₃) δ 8.33 (s, 2H), 8.03 (s, 1H), 7.21 (d, J=8.7 Hz, 2H), 7.05 (d, J=9.2 Hz, 2H), 4.48-4.40 (m, 3H), 4.31-4.25 (m, 1H), 4.18 (dd, J=12.4, 4.5 Hz, 1H), 3.99-3.92 (m, 1H), 3.90-3.84 (m, 1H), 3.75 (s, 2H), 3.60 (dd, J=12.9, 3.5 Hz, 1H), 3.46 (ddd, J=13.0, 6.6, 3.5 Hz, 1H), 3.40 (dd, J=4.4, 2.6 Hz, 1H), 3.28-3.21 (m, 1H), 3.20-3.11 (m, 1H), 1.01 (d, J=6.5 Hz, 3H). ESI-LR: 535.20 [M+1]⁺.

### Example 20

### (3S)—N-((6-(2-methyl-4-(4-(trifluoromethoxy)phenyl)piperazin-1-yl)pyrimidin-3-yl)methyl)-7-nitro-3,4-dihydro-2H-imidazo[2,1-b][1,3]oxazin-3-amine (compound 20)

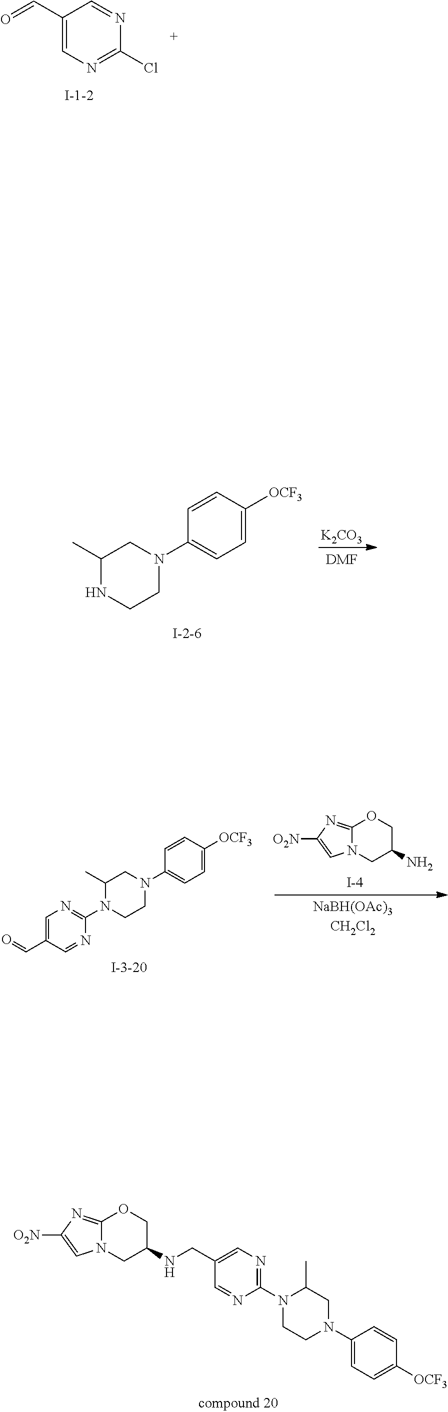

(1) 3-methyl-1-(4-(trifluoromethoxy)phenyl)piperazine I-2-6 (200 mg, 0.77 mmol) and 2-chloro-5-formylpyrimidine I-1-2 (130 mg, 0.92 mmol) were used as raw materials, and the operation method was the same as the method of (1) in Example 1, giving intermediate I-3-20 (185 mg, yield: 65.3%).

Intermediate I-3-20: ¹H-NMR (400 MHz, CDCl₃) δ 9.78 (s, 1H), 9.79 (s, 1H), 8.75 (s, 2H), 7.18-7.14 (m, 2H), 6.95-6.92 (m, 2H), 4.80-4.47 (m, 3H), 3.25-3.10 (m, 4H), 1.17 (d, J=6.5 Hz, 3H).

(2) Intermediate I-3-20 (183 mg, 0.50 mmol) and I-4 (92 mg, 0.50 mmol) were used as raw materials, and the operation method was the same as the method of (2) in Example 1, giving pale yellow compound 20 (131 mg, yield: 49.2%).

Compound 20: ¹H-NMR (400 MHz, CDCl₃) δ 8.33 (s, 2H), 8.03 (s, 1H), 7.20 (d, J=8.5 Hz, 2H), 7.03 (d, J=9.3 Hz, 2H), 4.89-4.82 (m, 1H), 4.40-4.30 (m, 1H), 4.16 (dd, J=12.8, 4.0 Hz, 1H), 3.97 (dd, J=12.7, 3.2 Hz, 1H), 3.70 (d, J=11.9 Hz, 1H), 3.61 (d, J=10.7 Hz, 3H), 3.29-3.20 (m, 3H), 2.94-2.90 (m, 1H), 2.78-2.64 (m, 2H), 1.20 (d, J=6.6 Hz, 3H). ESI-LR: 535.20 [M+1]⁺.

### Example 21

### (S)-7-nitro-N-((6-(4-(4-(trifluoromethoxy)phenyl)piperidin-1-yl)pyrimidin-3-yl) methyl)-3,4-dihydro-2H-imidazo[2,1-b][1,3]oxazin-3-amine (compound 21)

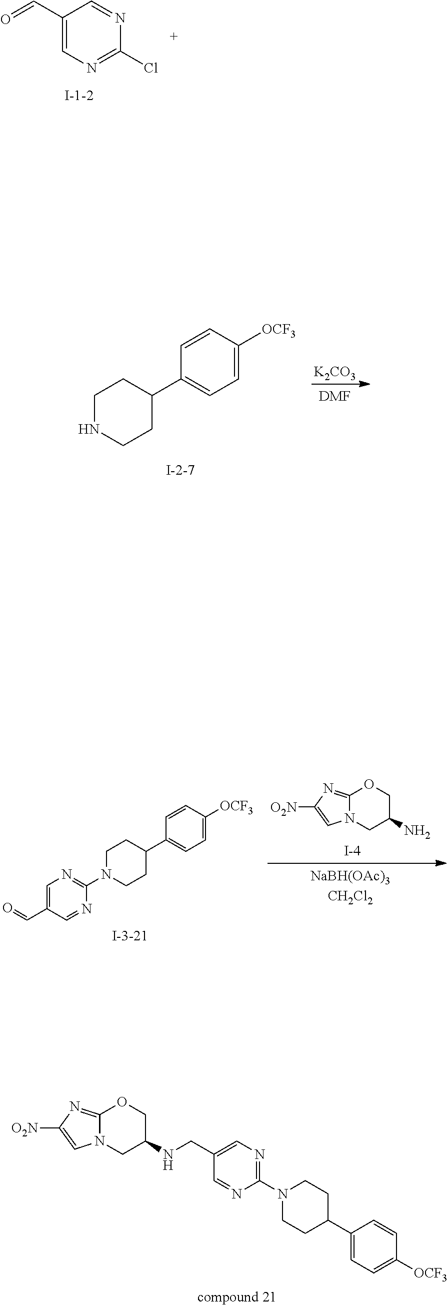

(1) 4-(4-(trifluoromethoxy)phenyl)piperidine I-2-7 (188 mg, 0.77 mmol) and 2-chloro-5-formylpyrimidine I-1-2 (130 mg, 0.92 mmol) were used as raw materials, and the operation method was the same as the method of (1) in Example 1, giving intermediate I-3-21 (231 mg, yield: 85.4%).

Intermediate I-3-21: ¹H-NMR (400 MHz, CDCl₃) δ 9.78 (s, 1H), 9.79 (s, 1H), 8.75 (s, 2H), 7.18-7.14 (m, 2H), 6.95-6.92 (m, 2H), 4.00-3.90 (m, 2H), 3.76-3.73 (m, 2H), 3.68-3.57 (m, 1H), 2.00-1.89 (m, 2H), 1.82-1.78 (m, 2H).

(2) Intermediate I-3-21 (210 mg, 0.60 mmol) and I-4 (110 mg, 0.60 mmol) were used as raw materials, and the operation method was the same as the method of (2) in Example 1, giving pale yellow compound 21 (149 mg, yield: 48.7%).

Compound 21: ¹H-NMR (400 MHz, CDCl₃) δ 8.30 (s, 2H), 8.01 (s, 1H), 7.36 (d, J=8.7 Hz, 2H), 7.25 (d, J=9.2 Hz, 2H), 4.79 (d, J=12.9 Hz, 2H), 4.41-4.29 (m, 2H), 4.13 (dd, J=12.7, 4.0 Hz, 1H), 3.98-3.91 (m, 1H), 3.61 (s, 2H), 2.97-2.81 (m, 4H), 1.85-1.81 (m, 2H), 1.52-1.45 (m, 2H). ESI-LR: 520.18 [M+1]⁺.

### Example 22

### (S)-1-(5-(((7-nitro-3,4-dihydro-2H-imidazo[2,1-b][1,3]oxazin-3-yl)amino)methyl)pyrimidin-2-yl)-4-(4-(trifluoromethoxy)phenyl)piperidin-4-ol (compound 22)

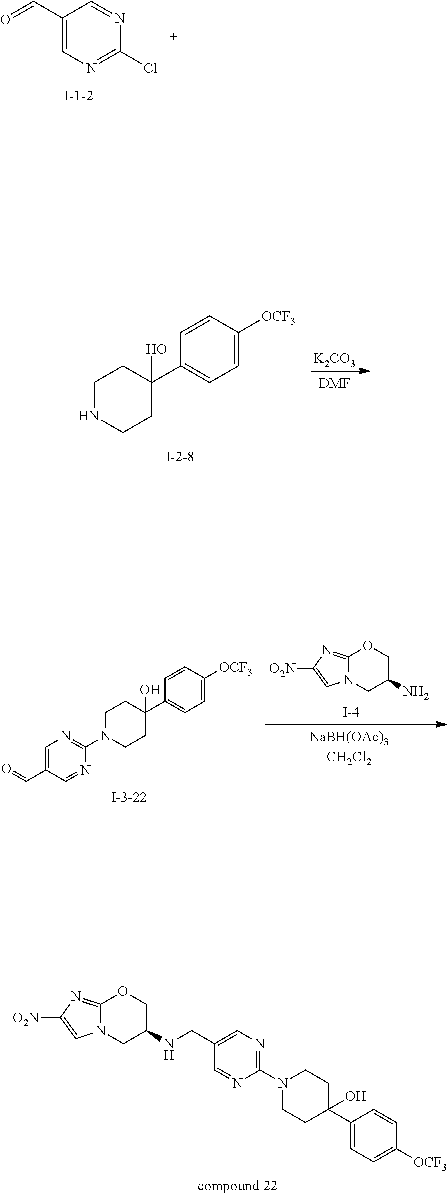

(1) 4-(4-(trifluoromethoxy)phenyl)piperidin-4-ol I-2-8 (200 mg, 0.77 mmol) and 2-chloro-5-formylpyrimidine I-1-2 (130 mg, 0.92 mmol) were used as raw materials, and the operation method was the same as the method of (1) in Example 1, giving intermediate I-3-22 (191 mg, yield: 67.8%).

Intermediate I-3-22: ¹H-NMR (400 MHz, CDCl₃) δ 9.78 (s, 1H), 9.79 (s, 1H), 8.75 (s, 2H), 7.18-7.14 (m, 2H), 6.95-6.92 (m, 2H), 4.00-3.90 (m, 2H), 3.76-3.73 (m, 2H), 2.14-2.03 (m, 2H), 1.96-1.91 (m, 2H).

(2) Intermediate I-3-22 (183 mg, 0.50 mmol) and I-4 (92 mg, 0.50 mmol) were used as raw materials, and the operation method was the same as the method of (2) in Example 1, giving pale yellow compound 22 (103 mg, yield: 38.5%).

Compound 22: ¹H-NMR (400 MHz, CDCl₃) δ 8.31 (s, 2H), 8.02 (s, 1H), 7.37 (d, J=8.5 Hz, 2H), 7.24 (d, J=9.3 Hz, 2H), 4.34 (dt, J=11.2, 8.0 Hz, 2H), 4.13-4.09 (m, 1H), 3.98-3.79 (m, 3H), 3.59 (d, J=11.6 Hz, 2H), 3.38 (s, 1H), 3.26 (t, J=12.6 Hz, 2H), 2.23-2.19 (m, 2H), 1.88-1.84 (m, 2H). ESI-LR: 536.18 [M+1]⁺.

### Example 23

### (S)—N-((6-(4-methoxy-4-(4-(trifluoromethoxy)phenyl)piperidin-1-yl)pyrimidin-3-yl)methyl)-7-nitro-3,4-dihydro-2H-imidazo[2,1-b][1,3]oxazin-3-amine (compound 23)

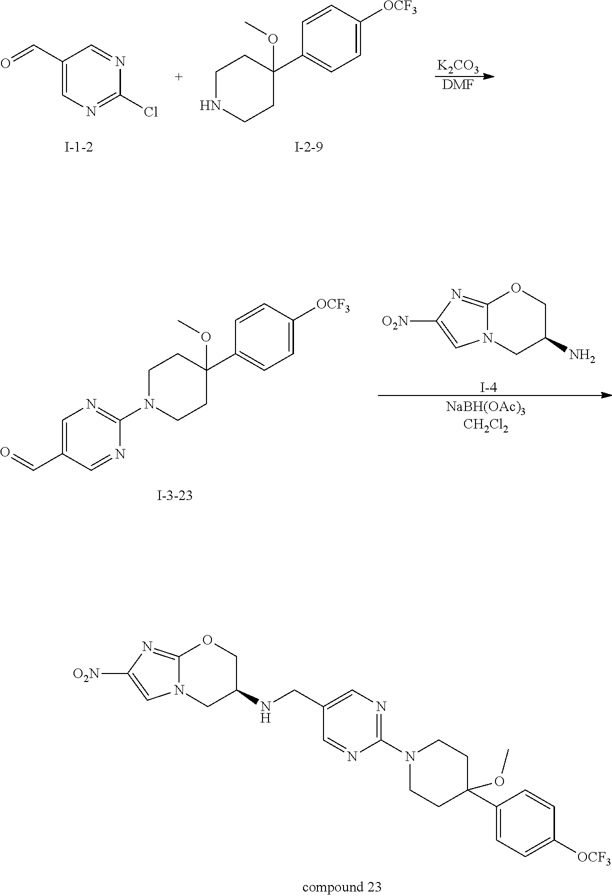

(1) 4-methoxy-4-(4-(trifluoromethoxy)phenyl)piperidine I-2-9 (212 mg, 0.77 mmol) and 2-chloro-5-formylpyrimidine I-1-2 (130 mg, 0.92 mmol) were used as raw materials, and the operation method was the same as the method of (1) in Example 1, giving intermediate I-3-23 (208 mg, yield: 70.9%).

Intermediate I-3-23: ¹H-NMR (400 MHz, CDCl₃) δ 9.78 (s, 1H), 9.79 (s, 1H), 8.75 (s, 2H), 7.18-7.14 (m, 2H), 6.95-6.92 (m, 2H), 4.00-3.90 (m, 2H), 3.76-3.73 (m, 2H), 3.57 (s, 3H), 2.12-2.01 (m, 2H), 1.94-1.89 (m, 2H).

(2) Intermediate I-3-23 (190 mg, 0.50 mmol) and I-4 (92 mg, 0.50 mmol) were used as raw materials, and the operation method was the same as the method of (2) in Example 1, giving pale yellow compound 23 (115 mg, yield: 42.1%).

Compound 23: ¹H-NMR (400 MHz, CDCl₃) δ 8.33 (s, 2H), 8.03 (s, 1H), 7.38 (d, J=8.5 Hz, 2H), 7.27 (d, J=9.3 Hz, 2H), 4.40 (dt, J=11.2, 8.0 Hz, 2H), 4.11-4.07 (m, 1H), 4.00-3.81 (m, 6H), 3.59 (d, J=11.6 Hz, 2H), 3.38 (s, 1H), 3.26-3.20 (m, 2H), 2.23-2.19 (m, 2H), 1.88-1.84 (m, 2H). ESI-LR: 550.19 [M+1]⁺.

### Example 24

### (S)-1-(5-(((7-nitro-3,4-dihydro-2H-imidazo[2,1-b][1,3]oxazin-3-yl)amino)methyl)pyrimidin-2-yl)-4-(4-(trifluoromethoxy)phenyl)piperidine-4-carbonitrile (compound 24)

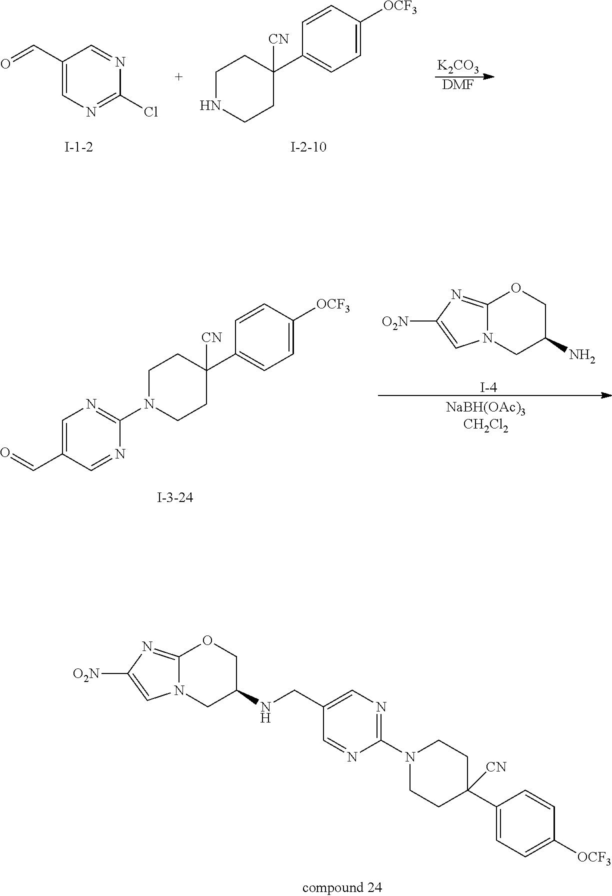

(1) 4-(4-(trifluoromethoxy)phenyl)piperidine-4-carbonitrile I-2-10 (208 mg, 0.77 mmol) and 2-chloro-5-formylpyrimidine I-1-2 (130 mg, 0.92 mmol) were used as raw materials, and the operation method was the same as the method of (1) in Example 1, giving intermediate I-3-24 (227 mg, yield: 78.5%).

Intermediate I-3-24: ¹H-NMR (400 MHz, CDCl₃) δ 9.78 (s, 1H), 9.79 (s, 1H), 8.75 (s, 2H), 7.18-7.14 (m, 2H), 6.95-6.92 (m, 2H), 4.03-3.91 (m, 2H), 3.77-3.74 (m, 2H), 2.32-2.23 (m, 2H), 2.14-2.09 (m, 2H).

(2) Intermediate I-3-24 (225 mg, 0.60 mmol) and I-4 (110 mg, 0.60 mmol) were used as raw materials, and the operation method was the same as the method of (2) in Example 1, giving pale yellow compound 24 (139 mg, yield: 42.8%).

Compound 24: ¹H-NMR (400 MHz, CDCl₃) δ 8.32 (s, 2H), 8.00 (s, 1H), 7.40 (d, J=8.5 Hz, 2H), 7.31 (d, J=9.3 Hz, 2H), 4.43 (dt, J=11.2, 8.0 Hz, 2H), 4.13-4.08 (m, 1H), 4.03-3.92 (m, 3H), 3.61 (d, J=11.6 Hz, 2H), 3.42 (s, 1H), 3.32-3.25 (m, 2H), 2.94-2.87 (m, 2H), 2.30-2.25 (m, 2H). ESI-LR: 545.18 [M+1]⁺.

### Example 25

### (6S)-2-nitro-N-((6-(5-(4-(trifluoromethoxy)phenyl)hexahydropyrrolo[3,4-c]pyrrol-2(1H)-yl)pyrimidin-3-yl)methyl)-6,7-dihydro-5H-imidazo[2,1-b][1,3]oxazin-6-amine (compound 25)

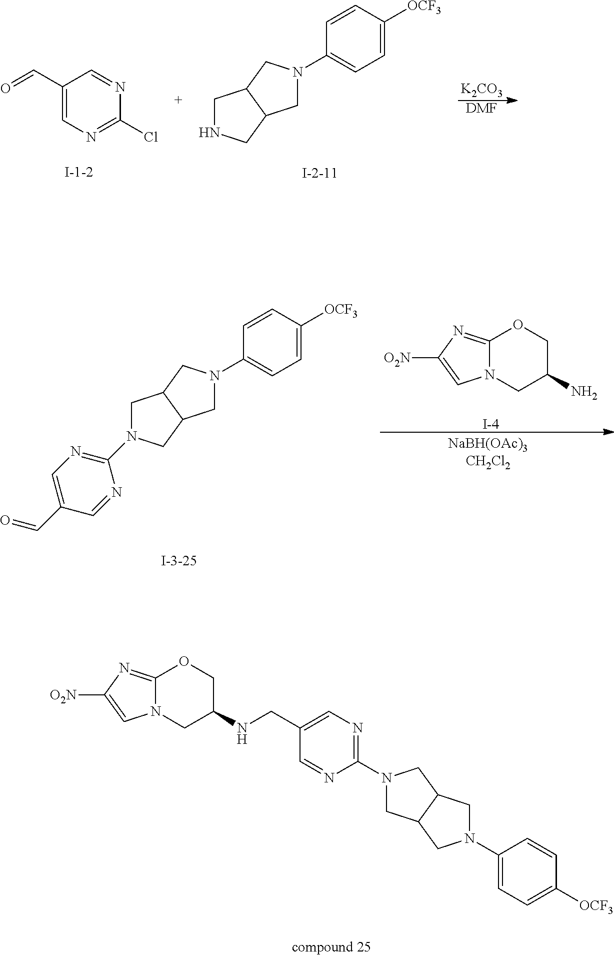

(1) 2-(4-(trifluoromethoxy)phenyl)octahydropyrrolo[3, 4]pyrrole I-2-11 (209 mg, 0.77 mmol) and 2-chloro-5-formylpyrimidine I-1-2 (130 mg, 0.92 mmol) were used as raw materials, and the operation method was the same as the method of (1) in Example 1, giving intermediate I-3-25 (241 mg, yield: 82.7%).

Intermediate I-3-25: ¹H-NMR (400 MHz, CDCl₃) δ 9.78 (s, 1H), 9.79 (s, 1H), 8.75 (s, 2H), 7.18-7.14 (m, 2H), 6.95-6.92 (m, 2H), 3.83-3.71 (m, 4H), 3.49-3.35 (m, 4H), 3.18 (s, 2H).

(2) Intermediate I-3-25 (226 mg, 0.60 mmol) and I-4 (110 mg, 0.60 mmol) were used as raw materials, and the operation method was the same as the method of (2) in Example 1, giving pale yellow compound 25 (166 mg, yield: 50.7%).

Compound 25: ¹H-NMR (400 MHz, CDCl₃) δ 8.33 (s, 2H), 8.01 (s, 1H), 7.38 (d, J=8.5 Hz, 2H), 7.30 (d, J=9.3 Hz, 2H), 4.41-4.32 (m, 2H), 4.12 (dd, J=12.3, 4.5 Hz, 1H), 3.90 (dd, J=12.4, 3.4 Hz, 1H), 3.83-3.71 (m, 4H), 3.63-3.55 (m, 2H), 3.49-3.35 (m, 4H), 3.27 (dd, J=9.5, 3.8 Hz, 2H), 3.18 (s, 2H). ESI-LR: 547.20 [M+1]⁺.

### Example 26

### (6S)-2-nitro-N-((6-(5-(4-(trifluoromethoxy)phenyl)-2,5-diazabicyclo[2.2.1]heptan-2-yl)pyrimidin-3-yl)methyl)-6,7-dihydro-5H-imidazo[2,1-b][1,3]oxazin-6-amine (compound 26)

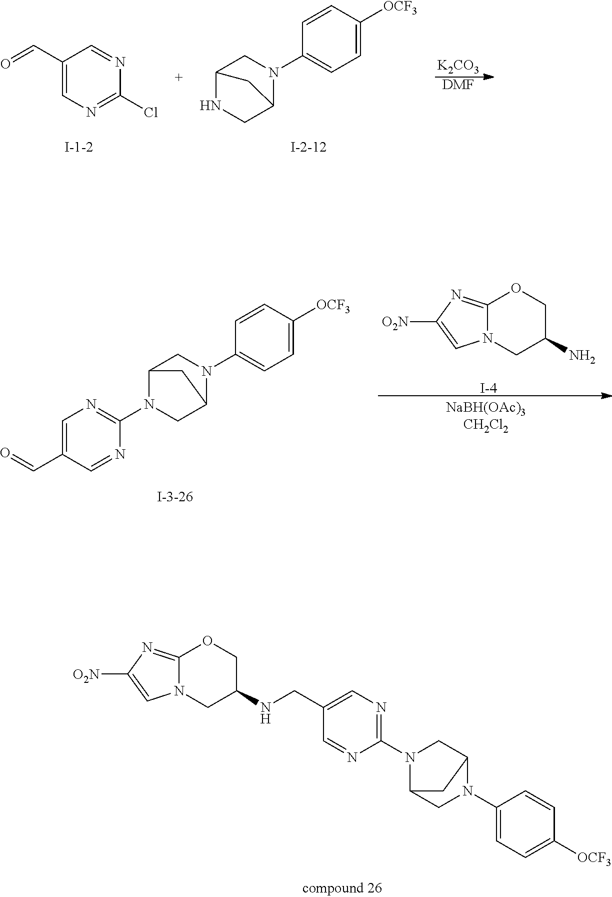

(1) 2-(4-(trifluoromethoxy)phenyl)-2,5-diazabicyclo[2.2.1]heptane I-2-12 (198 mg, 0.77 mmol) and 2-chloro-5-formylpyrimidine I-1-2 (130 mg, 0.92 mmol) were used as raw materials, and the operation method was the same as the method of (1) in Example 1, giving intermediate I-3-26 (201 mg, yield: 71.7%).

Intermediate I-3-26: ¹H-NMR (400 MHz, CDCl₃) δ 9.78 (s, 1H), 9.79 (s, 1H), 8.75 (s, 2H), 7.18-7.14 (m, 2H), 6.95-6.92 (m, 2H), 3.71-3.65 (m, 3H), 3.31-3.25 (m, 3H), 1.78-1.73 (m, 1H), 1.53-1.47 (m, 1H).

(2) Intermediate I-3-26 (181 mg, 0.50 mmol) and I-4 (84 mg, 0.50 mmol) were used as raw materials, and the operation method was the same as the method of (2) in Example 1, giving pale yellow compound 26 (110 mg, yield: 42.5%).

Compound 26: ¹H-NMR (400 MHz, CDCl₃) δ 8.32 (s, 2H), 8.01 (s, 1H), 7.38 (d, J=8.5 Hz, 2H), 7.27 (d, J=9.3 Hz, 2H), 4.40-4.38 (m, 1H), 4.32 (dd, J=12.0, 4.3 Hz, 1H), 4.13 (dd, J=12.3, 4.5 Hz, 1H), 3.90 (dd, J=12.2, 3.4 Hz, 1H), 3.86-3.76 (m, 2H), 3.70-3.63 (m, 3H), 3.40 (dd, J=4.7, 2.6 Hz, 1H), 3.30-3.24 (m, 3H), 1.77-1.72 (m, 1H), 1.52-1.49 (m, 1H). ESI-LR: 533.18 [M+1]⁺.

### Example 27

### (S)-2-nitro-N-((6-(2-(4-(trifluoromethoxy)phenyl)-2,7-diazaspiro[3.5]nonan-7-yl) pyrimidin-3-yl)methyl)-6,7-dihydro-5H-imidazo[2,1-b][1,3]oxazin-6-amine (compound 27)

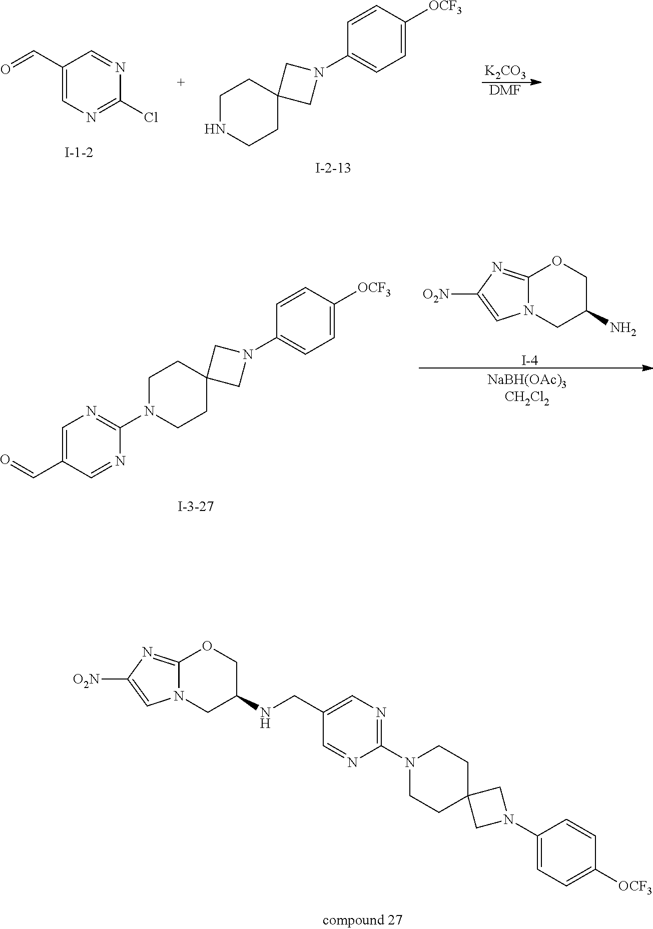

(1) 2-(4-(trifluoromethoxy)phenyl)-2,7-diazaspiro[3.5]nonane I-2-13 (220 mg, 0.77 mmol) and 2-chloro-5-formylpyrimidine I-1-2 (130 mg, 0.92 mmol) were used as raw materials, and the operation method was the same as the method of (1) in Example 1, giving intermediate I-3-27 (220 mg, yield: 73.1%).

Intermediate I-3-27: ¹H-NMR (400 MHz, CDCl₃) δ 9.78 (s, 1H), 9.79 (s, 1H), 8.75 (s, 2H), 7.18-7.14 (m, 2H), 6.95-6.92 (m, 2H), 5.21-4.61 (m, 4H), 3.57-3.50 (m, 4H), 1.59-1.51 (m, 4H).

(2) Intermediate I-3-27 (195 mg, 0.50 mmol) and I-4 (84 mg, 0.50 mmol) were used as raw materials, and the operation method was the same as the method of (2) in Example 1, giving pale yellow compound 27 (110 mg, yield: 39.6%).

Compound 27: ¹H-NMR (400 MHz, CDCl₃) δ 8.32 (s, 2H), 8.01 (s, 1H), 7.38 (d, J=8.5 Hz, 2H), 7.27 (d, J=9.3 Hz, 2H), 4.40 (dt, J=11.2, 8.0 Hz, 2H), 4.11-4.07 (m, 1H), 4.00-3.81 (m, 6H), 3.59-3.50 (m, 6H), 3.39 (s, 1H), 3.28-3.21 (m, 2H), 2.27-2.20 (m, 2H), 1.95-1.89 (m, 2H). ESI-LR: 561.21 [M+1]⁺.

### Example 28

### (6S)-2-nitro-N-((6-(3-(4-(trifluoromethoxy)phenoxy)-8-azabicyclo[3.2.1]octan-8-yl)pyrimidin-3-yl)methyl)-6,7-dihydro-5H-imidazo[2,1-b][1,3]oxazin-6-amine (compound 28)

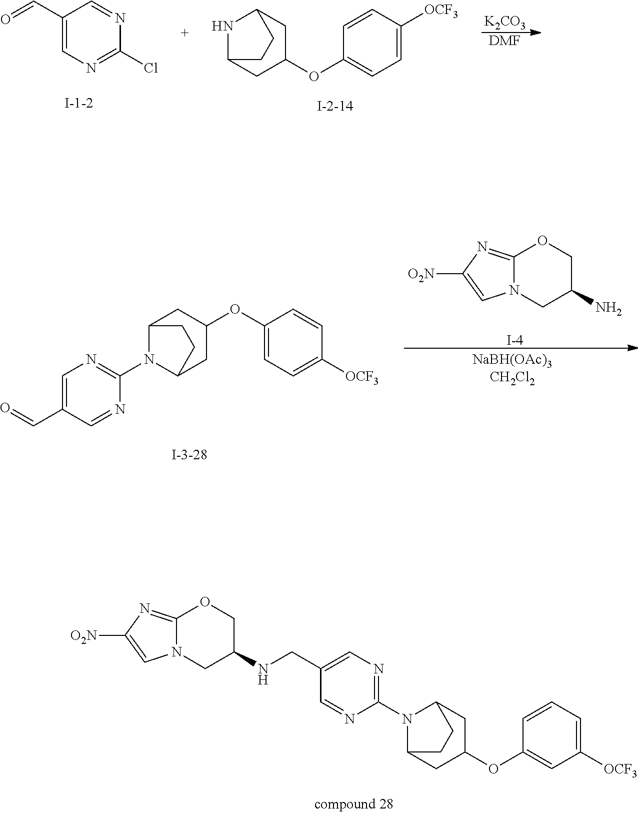

(1) 2-(4-(trifluoromethoxy)phenyl)-8-azabicyclo[3.2.1]octane I-2-14 (220 mg, 0.77 mmol) and 2-chloro-5-formylpyrimidine I-1-2 (130 mg, 0.92 mmol) were used as raw materials, and the operation method was the same as the method of (1) in Example 1, giving intermediate I-3-28 (214 mg, yield: 70.9%).

Intermediate I-3-28: ¹H-NMR (400 MHz, CDCl₃) δ 9.78 (s, 1H), 9.79 (s, 1H), 8.75 (s, 2H), 7.18-7.14 (m, 2H), 6.95-6.92 (m, 2H), 4.62-4.54 (m, 1H), 3.57-3.51 (m, 2H), 2.05-1.95 (m, 2H), 1.87-1.83 (m, 2H), 1.79-1.75 (m, 2H), 1.47-1.50 (m, 2H).

(2) Intermediate I-3-28 (196 mg, 0.50 mmol) and I-4 (84 mg, 0.50 mmol) were used as raw materials, and the operation method was the same as the method of (2) in Example 1, giving pale yellow compound 28 (128 mg, yield: 45.7%).

Compound 28: ¹H-NMR (400 MHz, CDCl₃) δ 8.11 (s, 1H), 7.45 (dd, J=8.7, 2.4 Hz, 1H), 7.36 (s, 1H), 7.15 (d, J=8.6 Hz, 2H), 6.94-6.89 (m, 2H), 6.67 (d, J=8.7 Hz, 1H), 4.73-4.50 (m, 1H), 4.42-4.30 (m, 2H), 4.13 (dd, J=12.4, 4.5 Hz, 1H), 3.87-3.79 (m, 3H), 3.81-3.72 (m, 2H), 3.42-3.37 (m, 1H), 2.07-1.98 (m, 2H), 1.88-1.80 (m, 2H), 1.70-1.65 (m, 2H), 1.45-1.48 (m, 2H). ESI-LR: 562.19 [M+1]⁺.

### Example 29

### (S)-2-nitro-N-((2-(4-(4-(trifluoromethoxy)phenyl)-1,4-diazocyclohept-1-yl)pyrimidin-5-yl)methyl)-6,7-dihydro-5H-imidazo[2,1-b][1,3]oxazin-6-amine (compound 29)

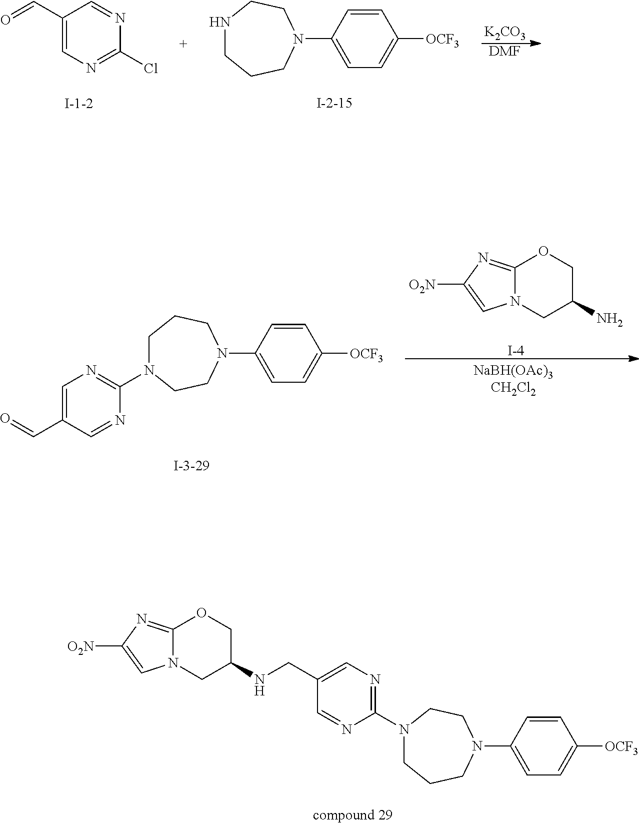

(1) 1-(4-(trifluoromethoxy)phenyl)-1,4-diazaheptane I-2-15 (200 mg, 0.77 mmol) (reference: WO 2005100365) and 2-chloro-5-formylpyrimidine I-1-2 (130 mg, 0.92 mmol) were used as raw materials, and the operation method was the same as the method of (1) in Example 1, giving intermediate I-3-29 (137 mg, yield: 68.7%).

Intermediate I-3-29: ¹H-NMR (400 MHz, CDCl₃) δ 9.78 (s, 1H), 9.79 (s, 1H), 8.75 (s, 2H), 7.18-7.14 (m, 2H), 6.95-6.92 (m, 2H), 4.58-4.54 (m, 2H), 4.18-4.14 (m, 4H), 3.27-3.24 (m, 2H), 2.73-2.69 (m, 2H).

(2) Intermediate I-3-29 (130 mg, 0.50 mmol) and I-4 (84 mg, 0.50 mmol) were used as raw materials, and the operation method was the same as the method of (2) in Example 1, giving pale yellow compound 29 (134 mg, yield: 50.5%).

Compound 29: ¹H-NMR (400 MHz, CDCl₃) δ 8.33 (s, 2H), 8.03 (s, 1H), 7.21 (d, J=8.7 Hz, 2H), 7.05 (d, J=9.2 Hz, 2H), 4.58-4.54 (m, 2H), 4.41-4.35 (m, 2H), 4.14 (dd, J=12.3, 4.5 Hz, 1H), 3.92 (dd, J=12.2, 3.4 Hz, 1H), 3.82-3.70 (m, 4H), 3.62 (s, 2H), 3.31-3.21 (m, 3H), 2.73-2.69 (m, 2H). ESI-LR: 535.20 [M+1]⁺.

### Example 30

### (S)-2-nitro-N-((2-(4-((4-(trifluoromethoxy)phenyl)amino)piperidin-1-yl)pyrimidin-5-yl)methyl)-6,7-dihydro-5H-imidazo[2,1-b][1,3]oxazin-6-amine (compound 30)

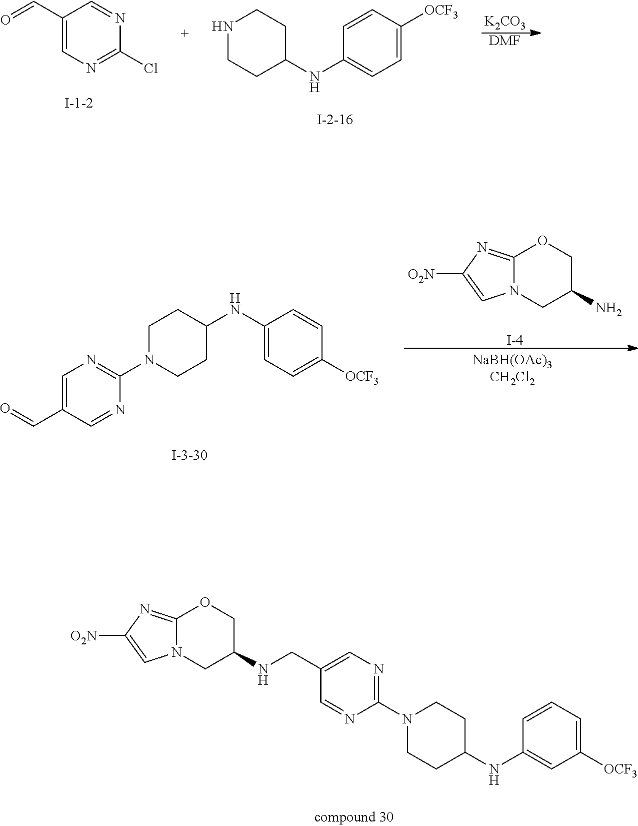

(1) N-(4-(trifluoromethylamino)phenoxy)piperidin-4-amine I-2-16 (200 mg, 0.77 mmol) (reference: WO 2011134296) and 2-chloro-5-formylpyrimidine I-1-2 (130 mg, 0.92 mmol) were used as raw materials, and the operation method was the same as the method of (1) in Example 1, giving intermediate I-3-30 (189 mg, yield: 67.3%).

Intermediate I-3-30: ¹H-NMR (400 MHz, CDCl₃) δ 9.79 (s, 1H), 8.75 (s, 2H), 7.07-7.03 (m, 2H), 6.84-6.81 (m, 2H), 4.02-3.92 (m, 2H), 3.57-3.51 (m, 3H), 1.85-1.75 (m, 2H), 1.78-1.74 (m, 2H).

(2) Intermediate I-3-30 (183 mg, 0.50 mmol) and I-4 (92 mg, 0.50 mmol) were used as raw materials, and the operation method was the same as the method of (2) in Example 1, giving pale yellow compound 30 (186 mg, yield: 58.1%).

Compound 30: ¹H-NMR (400 MHz, CDCl₃) δ 8.30 (s, 2H), 8.03 (s, 1H), 7.28 (d, J=8.7 Hz, 2H), 7.09 (d, J=9.1 Hz, 2H), 4.51-4.40 (m, 2H), 4.37-4.34 (m, 2H), 4.17-4.13 (m, 1H), 3.98-3.95 (m, 1H), 3.60 (s, 2H), 3.26-3.22 (m, 2H), 3.10-3.04 (m, 2H), 1.95-1.91 (m, 2H), 1.30-1.21 (m, 2H). ESI-LR: 535.20 [M+1]⁺.

### Example 31

### (S)-2-nitro-N-((2-(4-(4-(trifluoromethyl)phenyl)piperazin-1-yl)pyrimidin-5-yl) methyl)-6,7-dihydro-5H-imidazo[2,1-b][1,3]oxazin-6-amine (compound 31)

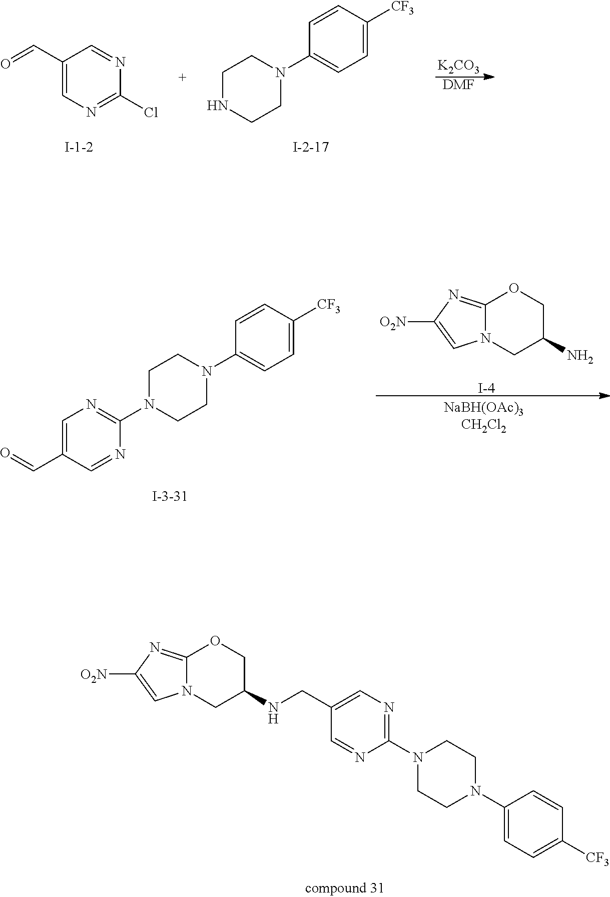

(1) 4-(4-(trifluoromethyl)phenyl)piperazine I-2-17 (177 mg, 0.77 mmol) (reference: J. Med. Chem. 2013, 56(24), 10158-10170) and 2-chloro-5-formylpyrimidine I-1-2 (130 mg, 0.92 mmol) were used as raw materials, and the operation method was the same as the method of (1) in Example 1, giving intermediate I-3-31 (226 mg, yield: 87.6%).

Intermediate I-3-31: ¹H-NMR (400 MHz, CDCl₃) δ 9.79 (s, 1H), 8.75 (s, 2H), 7.81-7.77 (m, 2H), 6.99-6.96 (m, 2H), 4.18-4.15 (m, 4H), 3.30-3.25 (m, 4H).

(2) Intermediate I-3-31 (201 mg, 0.60 mmol) and I-4 (110 mg, 0.60 mmol) were used as raw materials, and the operation method was the same as the method of (2) in Example 1, giving pale yellow compound 31 (168 mg, yield: 55.8%).

Compound 31: ¹H-NMR (400 MHz, CDCl₃) δ 8.33 (s, 2H), 8.03 (s, 1H), 7.84 (d, J=8.7 Hz, 2H), 7.09 (d, J=9.2 Hz, 2H), 4.41-4.35 (m, 2H), 4.14 (dd, J=12.3, 4.5 Hz, 1H), 3.92 (dd, J=12.2, 3.4 Hz, 1H), 3.85-3.73 (m, 4H), 3.62 (s, 2H), 3.34-3.23 (m, 5H). ESI-LR: 505.18 [M+1]⁺.

### Example 32

### (S)—N-((2-(4-(4-fluoro-3-methylphenyl)piperazin-1-yl)pyrimidin-5-yl)methyl)-2-nitro-6,7-dihydro-5H-imidazo[2,1-b][1,3]oxazin-6-amine (compound 32)

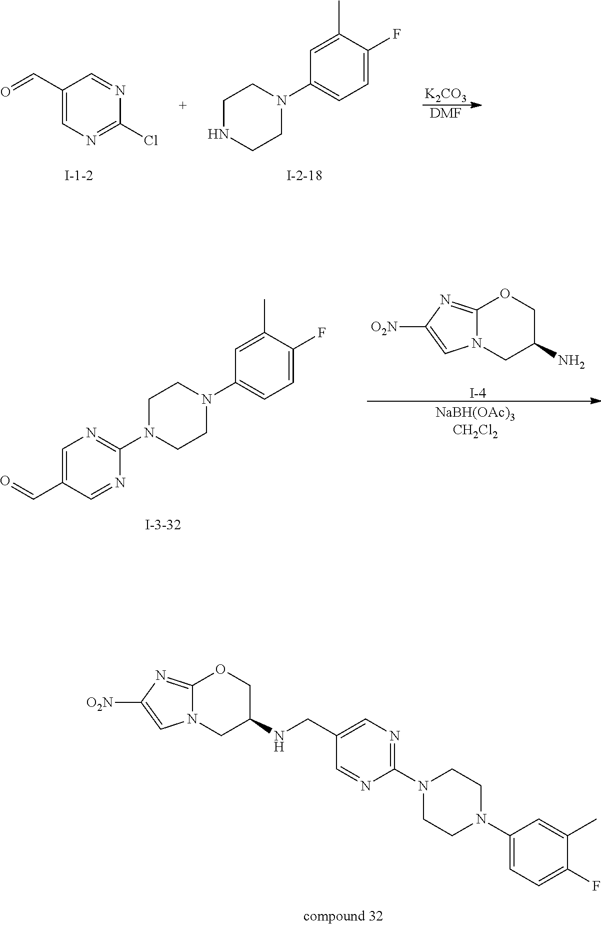

(1) 1-(4-fluoro-3-methylphenyl)piperazine I-2-18 (149 mg, 0.77 mmol) (reference: Letters in organic chemistry, 2011, 8(9), 628-630) and 2-chloro-5-formylpyrimidine I-1-2 (130 mg, 0.92 mmol) were used as raw materials, and the operation method was the same as the method of (1) in Example 1, giving intermediate I-3-32 (185 mg, yield: 80.4%).

Intermediate I-3-32: ¹H-NMR (400 MHz, CDCl₃) δ 9.79 (s, 1H), 8.75 (s, 2H), 7.31-7.27 (m, 1H), 6.97 (s, 1H), 6.82 (d, J=8.8 Hz, 1H), 4.18-4.15 (m, 4H), 3.30-3.25 (m, 4H), 2.37 (s, 3H).

(2) Intermediate I-3-32 (180 mg, 0.60 mmol) and I-4 (110 mg, 0.60 mmol) were used as raw materials, and the operation method was the same as the method of (2) in Example 1, giving pale yellow compound 32 (147 mg, yield: 52.7%).

Compound 32: ¹H-NMR (400 MHz, CDCl₃) δ 8.33 (s, 2H), 8.03 (s, 1H), 7.31-7.27 (m, 1H), 6.97 (s, 1H), 6.82 (d, J=8.8 Hz, 1H), 4.41-4.35 (m, 2H), 4.14 (dd, J=12.3, 4.5 Hz, 1H), 3.92 (dd, J=12.2, 3.4 Hz, 1H), 3.85-3.73 (m, 4H), 3.62 (s, 2H), 3.34-3.23 (m, 5H), 2.37 (s, 3H). ESI-LR: 469.20 [M+1]⁺.

### Example 33

### (S)—N-((2-(4-(6-methoxypyridin-3-yl)piperazin-1-yl)pyrimidin-5-yl)methyl)-2-nitro-6,7-dihydro-5H-imidazo[2,1-b][1,3]oxazin-6-amine (compound 33)

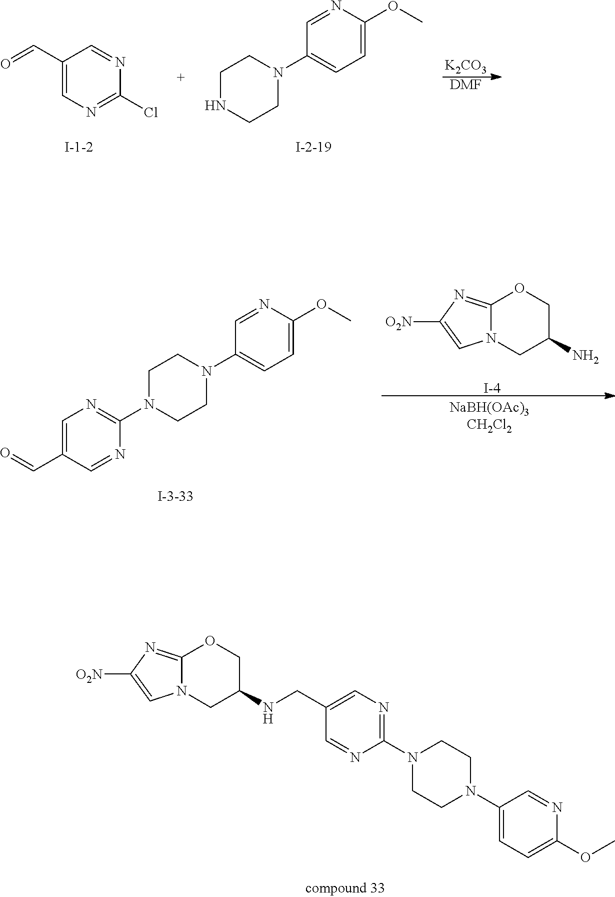

(1) 1-(6-methoxypyridin-3-yl)piperazine I-2-19 (194 mg, 1.0 mmol) (reference: WO 2010146083) and 2-chloro-5-formylpyrimidine I-1-2 (171 mg, 1.2 mmol) were used as raw materials, and the operation method was the same as the method of (1) in Example 1, giving intermediate I-3-33 (265 mg, yield: 88.5%).

Intermediate I-3-33: ¹H-NMR (400 MHz, CDCl₃) δ 9.79 (s, 1H), 8.75 (s, 2H), 7.15 (dd, J=8.8 Hz, 2.0 Hz, 1H), 6.97 (s, 1H), 6.82 (d, J=8.8 Hz, 1H), 4.18-4.15 (m, 4H), 3.63 (s, 3H), 3.30-3.25 (m, 4H).

(2) Intermediate I-3-33 (260 mg, 0.87 mmol) and I-4 (160 mg, 0.87 mmol) were used as raw materials, and the operation method was the same as the method of (2) in Example 1, giving pale yellow compound 33 (240 mg, yield: 60.0%).

Compound 33: ¹H-NMR (400 MHz, CDCl₃) δ 8.33 (s, 2H), 8.03 (s, 1H), 7.15 (dd, J=8.8 Hz, 2.0 Hz, 1H), 6.97 (s, 1H), 6.82 (d, J=8.8 Hz, 1H), 4.41-4.35 (m, 2H), 4.14 (dd, J=12.3, 4.5 Hz, 1H), 3.92 (dd, J=12.2, 3.4 Hz, 1H), 3.85-3.73 (m, 4H), 3.65 (s, 3H), 3.62 (s, 2H), 3.34-3.23 (m, 5H). ESI-LR: 468.20 [M+1]⁺.

### Example 34

### (S)-2-nitro-N-((2-(4-(5-(trifluoromethyl)pyrimidin-2-yl)piperazin-1-yl)pyrimidin-5-yl)methyl)-6,7-dihydro-5H-imidazo[2,1-b][1,3]oxazin-6-amine (compound 34)

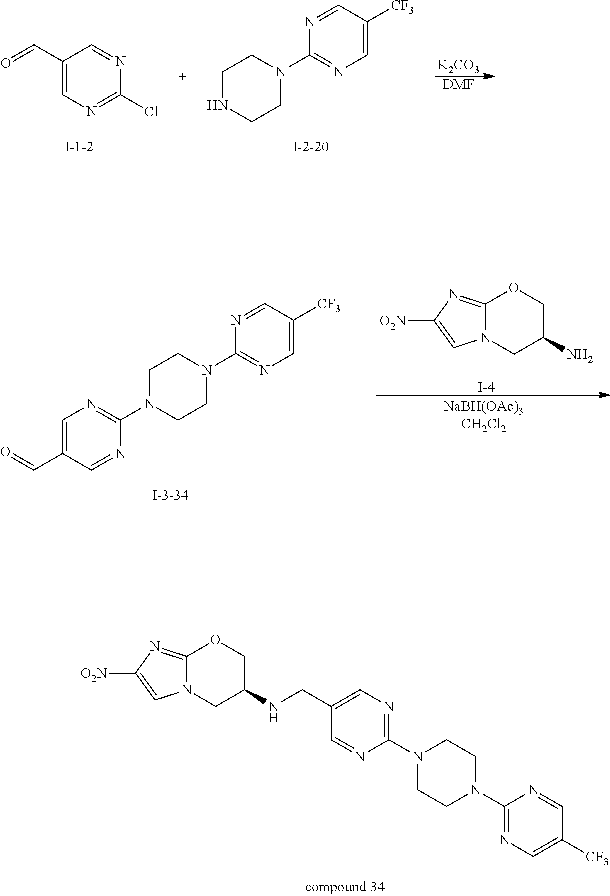

(1) 1-(4-fluoro-3-methylphenyl)piperazine I-2-20 (232 mg, 1.0 mmol) (reference: J. Med. Chem. 2010, 53(12), 4603-4614) and 2-chloro-5-formylpyrimidine I-1-2 (171 mg, 1.2 mmol) were used as raw materials, and the operation method was the same as the method of (1) in Example 1, giving intermediate I-3-34 (230 mg, yield: 68.0%).

Intermediate I-3-34: ¹H-NMR (400 MHz, CDCl₃) δ 9.79 (s, 1H), 8.95 (s, 2H), 8.75 (s, 2H), 4.18-4.15 (m, 4H), 3.30-3.25 (m, 4H).

(2) Intermediate I-3-34 (220 mg, 0.65 mmol) and I-4 (120 mg, 0.65 mmol) were used as raw materials, and the operation method was the same as the method of (2) in Example 1, giving pale yellow compound 34 (160 mg, yield: 48.6%).

Compound 34: ¹H-NMR (400 MHz, CDCl₃) δ 8.53 (s, 2H), 8.33 (s, 2H), 8.03 (s, 1H), 4.41-4.35 (m, 2H), 4.14 (dd, J=12.3, 4.5 Hz, 1H), 3.92 (dd, J=12.2, 3.4 Hz, 1H), 3.85-3.73 (m, 4H), 3.62 (s, 2H), 3.34-3.23 (m, 5H). ESI-LR: 507.18 [M+1]⁺.

### Example 35

### (S)-2-(4-(5-(((2-nitro-6,7-dihydro-5H-imidazo[2,1-b][1,3]oxazin-6-yl)amino)methyl)pyrimidin-2-yl)piperazin-1-yl)thiazole-4-carbonitrile (compound 35)

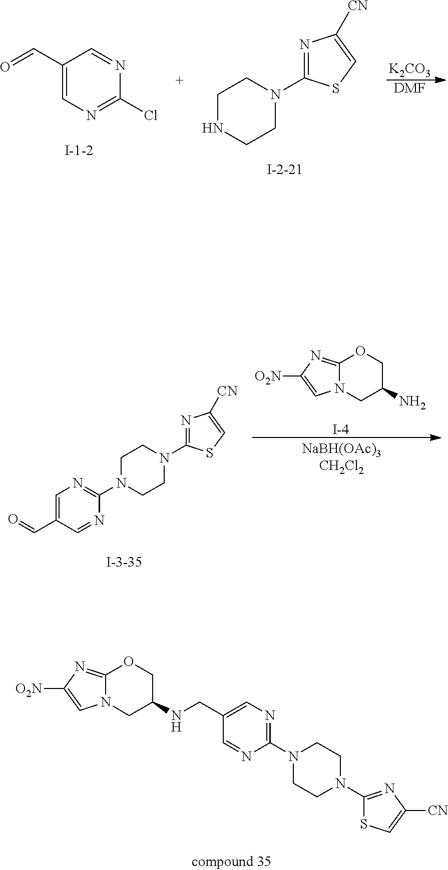

(1) 1-(4-fluoro-3-methylphenyl)piperazine I-2-21 (194 mg, 1.0 mmol) (reference: WO 2006072436) and 2-chloro-5-formylpyrimidine I-1-2 (171 mg, 1.2 mmol) were used as raw materials, and the operation method was the same as the method of (1) in Example 1, giving intermediate I-3-35 (249 mg, yield: 83.0%).

Intermediate I-3-35: ¹H-NMR (400 MHz, CDCl₃) δ 9.79 (s, 1H), 8.75 (s, 2H), 7.31 (s, 1H), 4.18-4.15 (m, 4H), 3.30-3.25 (m, 4H).

(2) Intermediate I-3-35 (240 mg, 0.80 mmol) and I-4 (147 mg, 0.80 mmol) were used as raw materials, and the operation method was the same as the method of (2) in Example 1, giving pale yellow compound 35 (208 mg, yield: 55.6%).

Compound 35: ¹H-NMR (400 MHz, CDCl₃) δ 8.33 (s, 2H), 8.03 (s, 1H), 7.31 (s, 1H), 4.41-4.35 (m, 2H), 4.14 (dd, J=12.3, 4.5 Hz, 1H), 3.92 (dd, J=12.2, 3.4 Hz, 1H), 3.85-3.73 (m, 4H), 3.62 (s, 2H), 3.34-3.23 (m, 5H). ESI-LR: 469.14 [M+1]⁺.

### Example 36

### (S)—N-(((4-methyl-2-(4-(4-(trifluoromethoxy)phenyl)piperazin-1-yl)pyrimidin-5-yl)methyl)-2-nitro-6,7-dihydro-5H-imidazo[2,1-b][1,3]oxazin-6-amine (compound 36)

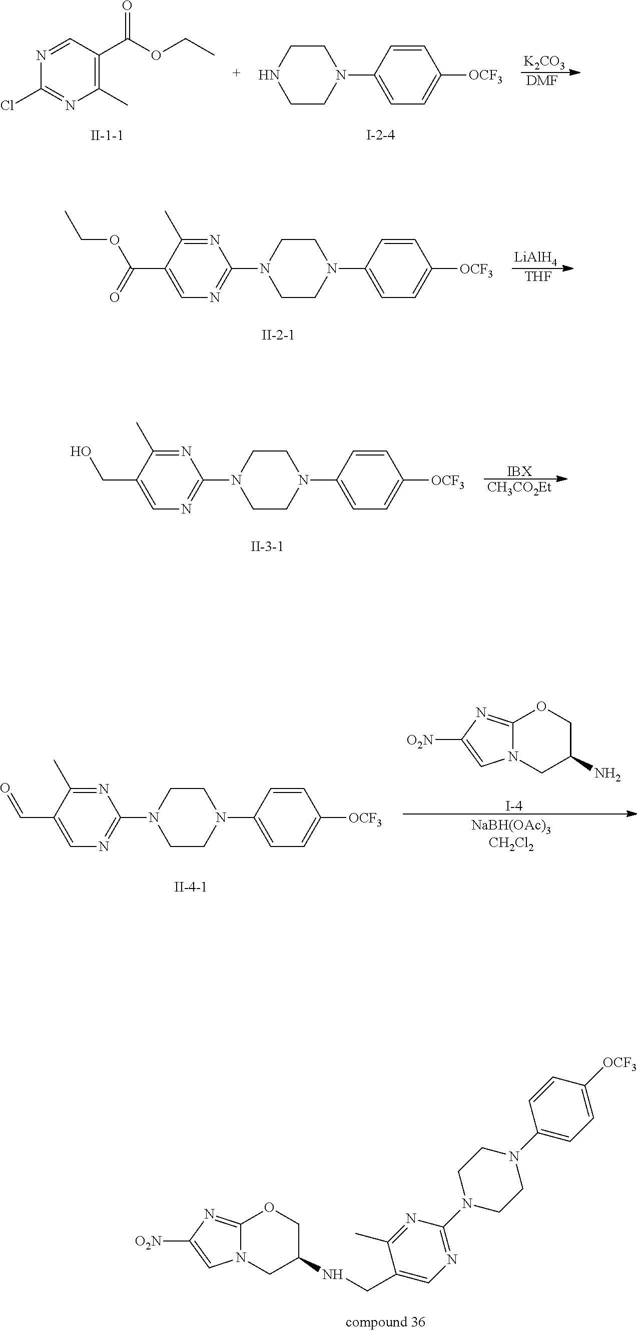

(1) 4-(4-(trifluoromethoxy)phenyl)piperazine I-2-4 (492 mg, 2.00 mmol) and ethyl 2-chloro-4-methylpyrimidin-5-carboxylate II-1-1 (440 mg, 2.20 mmol) (reference: WO 2012123467) were dissolved in DMF (8 mL), K₂CO₃ (828 mg, 6.00 mmol) was added to the solution dropwise and the mixture was reacted for 4 hours at 90° C. after the dropwise addition was completed. The reaction was completely cooled to room temperature, poured into ice water, extracted with ethyl acetate (20 mL*2), dried over anhydrous sodium sulfate, filtered, spin dried and purified by column chromatography (petroleum ether:ethyl acetate=4:1), giving intermediate II-2-1 (739 mg, yield: 90.2%) as a pale yellow solid.

Intermediate II-2-1: ¹H-NMR (400 MHz, CDCl₃) δ 8.57 (s, 1H), 7.18-7.14 (m, 2H), 6.95-6.92 (m, 2H), 4.43 (q, J=7.1 Hz, 2H), 4.10-4.07 (m, 4H), 3.27-3.24 (m, 4H), 2.32 (s, 3H), 1.43 (t, J=7.1 Hz, 3H).

(2) Intermediate II-2-1 (697 mg, 1.70 mmol) was dissolved in anhydrous tetrahydrofuran (10 mL), the solution was cooled to −30° C., lithium aluminum hydride (65 mg, 1.70 mmol) was added thereto, the reaction was carried out for 1.5 hours at this temperature, sodium sulfate decahydrate (200 mg) was added thereto, the reaction was slowly warmed to room temperature, stirred for half an hour and filtered, the solid was washed with tetrahydrofuran, and the organic phase was dried over anhydrous sodium sulfate, filtered and concentrated, giving intermediate II-3-1 (587 mg, yield: 93.9%) as a colorless oil, which was added directly to the next step reaction without purification. ESI-LR: 369.15 [M+1]⁺.

(3) Intermediate II-3-1 (478 mg, 1.30 mmol) was dissolved in ethyl acetate (10 mL), IBX (2-iodacyl benzoic acid, 546 mg, 1.95 mmol) was added to the solution and the mixture was warmed to 60° C. and reacted for 8 hours. After the reaction was completed, the mixture was cooled to room temperature, the insolubles were removed by filtration, the organic phase was directly spin dried and purified by column chromatography (petroleum ether:ethyl acetate=4:1), giving intermediate II-4-1 (349 mg, yield: 73.5%) as a pale yellow oil.

Intermediate II-4-1: ¹H-NMR (400 MHz, CDCl₃) δ 9.79 (s, 1H), 8.59 (s, 1H), 7.18-7.14 (m, 2H), 6.95-6.92 (m, 2H), 4.16-4.13 (m, 4H), 3.27-3.24 (m, 4H), 2.32 (s, 3H).

(4) Intermediate II-4-1 (260 mg, 0.71 mmol) and triethylamine (93 mg, 0.92 mmol) were dissolved in dichloromethane (10 mL), then raw material I-4 (131 mg, 0.71 mmol) was added to the solution, the mixture was reacted at room temperature overnight, NaBH(OAc)₃ (602 mg, 2.84 mmol) was added thereto, and the reaction was continued at room temperature overnight. A solution of sodium bicarbonate (10 mL) was added, the layers were separated, the aqueous layer was extracted with dichloromethane (20 mL*2), the dichloromethane layers were combined, washed with saturated sodium chloride solution, dried over anhydrous sodium sulfate and spin dried, and the residue was purified by column chromatography (dichloromethane:methanol=50:1), giving compound 36 (216 mg, yield: 57.2%) as a pale yellow powder.

Compound 36: ¹H-NMR (400 MHz, CDCl₃) δ 8.13 (s, 1H), 7.74 (s, 1H), 7.18-7.09 (m, 2H), 7.05-6.94 (m, 2H), 4.55-4.44 (m, 2H), 4.26 (dd, J=12.7, 4.1 Hz, 1H), 4.07 (dd, J=12.8, 4.0 Hz, 1H), 3.97-3.88 (m, 4H), 3.78-3.74 (m, 2H), 3.43-3.40 (m, 1H), 3.26-3.14 (m, 4H), 2.38 (s, 3H). ESI-LR: 535.20 [M+1]⁺.

### Example 37

### (S)—N-((4-methyl-2-(4-(4-(trifluoromethoxy)phenyl)piperazin-1-yl)pyrimidin-5-yl)ethyl)-2-nitro-6,7-dihydro-5H-imidazo[2,1-b][1,3]oxazin-6-amine (compound 37)

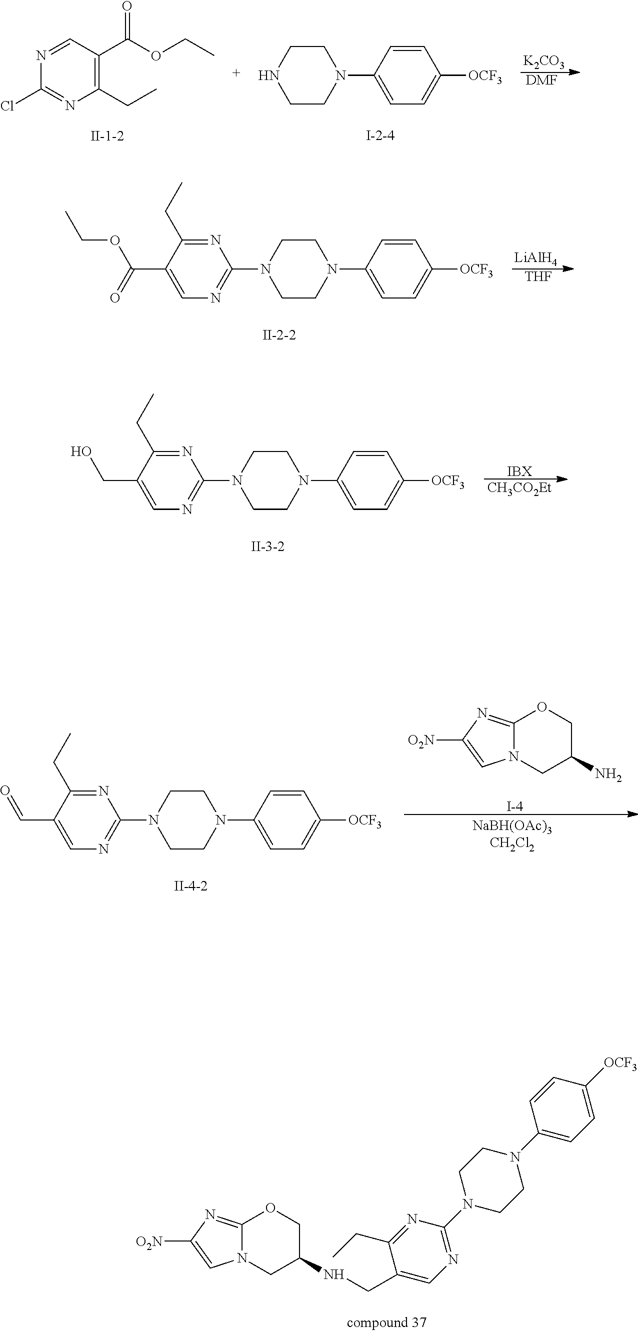

(1) 4-(4-(trifluoromethoxy)phenyl)piperazine I-2-4 (492 mg, 2.00 mmol) and ethyl 2-chloro-4-ethylpyrimidin-5-carboxylate II-1-2 (470 mg, 2.20 mmol) (reference: U.S. Pat. No. 5,935,966) were used as raw materials, and the operation method was the same as the method of (1) in Example 36, giving intermediate II-2-2 (741 mg, yield: 87.4%).

Intermediate II-2-2: ¹H-NMR (400 MHz, CDCl₃) δ 8.57 (s, 1H), 7.18-7.14 (m, 2H), 6.95-6.92 (m, 2H), 4.43 (q, J=7.1 Hz, 2H), 4.09-4.04 (m, 4H), 3.78 (q, J=7.2 Hz, 2H), 3.27-3.24 (m, 4H), 1.32-1.24 (m, 6H).

(2) Intermediate II-2-2 (720 mg, 1.70 mmol) and lithium aluminum hydride (65 mg, 1.70 mmol) were used as raw materials, and the operation method was the same as the method of (2) in Example 36, giving intermediate II-3-2 (534 mg, yield: 82.3%). Intermediate II-3-2: ESI-LR: 383.16 [M+1]⁺.

(3) Intermediate II-3-2 (496 mg, 1.30 mmol) and IBX (546 mg, 1.95 mmol) were used as raw materials, and the operation method was the same as the method of (3) in Example 33, giving intermediate II-4-2 (324 mg, yield: 65.7%) as a yellow oil.

Intermediate II-4-2: ¹H-NMR (400 MHz, CDCl₃) δ 9.79 (s, 1H), 8.59 (s, 1H), 7.18-7.14 (m, 2H), 6.95-6.92 (m, 2H), 4.16-4.13 (m, 4H), 3.78 (q, J=7.2 Hz, 2H), 3.27-3.24 (m, 4H), 1.28 (t, J=7.2 Hz, 3H).

(4) Intermediate II-4-2 (260 mg, 0.71 mmol) and I-4 (131 mg, 0.71 mmol) were used as raw materials, and the operation method was the same as the method of (4) in Example 36, giving compound 37 (169 mg, yield: 43.5%) as a pale yellow powder.

Compound 37: ¹H-NMR (400 MHz, CDCl₃) δ 8.09 (s, 1H), 7.40 (s, 1H), 7.13-7.06 (m, 2H), 6.99-6.91 (m, 2H), 4.47-4.38 (m, 2H), 4.18 (dd, J=12.7, 4.1 Hz, 1H), 3.97-3.88 (m, 5H), 3.78-3.74 (m, 2H), 3.43-3.40 (m, 1H), 3.26-3.18 (m, 4H), 2.72-2.65 (m, 2H) 1.28 (t, J=7.2 Hz, 3H). ESI-LR: 549.21 [M+1]⁺.

### Example 38

### (S)—N-((4-m ethoxy-2-(4-(4-(trifluoromethoxy)phenyl)piperazin-1-yl)pyrimidin-5-yl)methyl)-2-nitro-6,7-dihydro-5H-imidazo[2,1-b][1,3]oxazin-6-amine (compound 38)

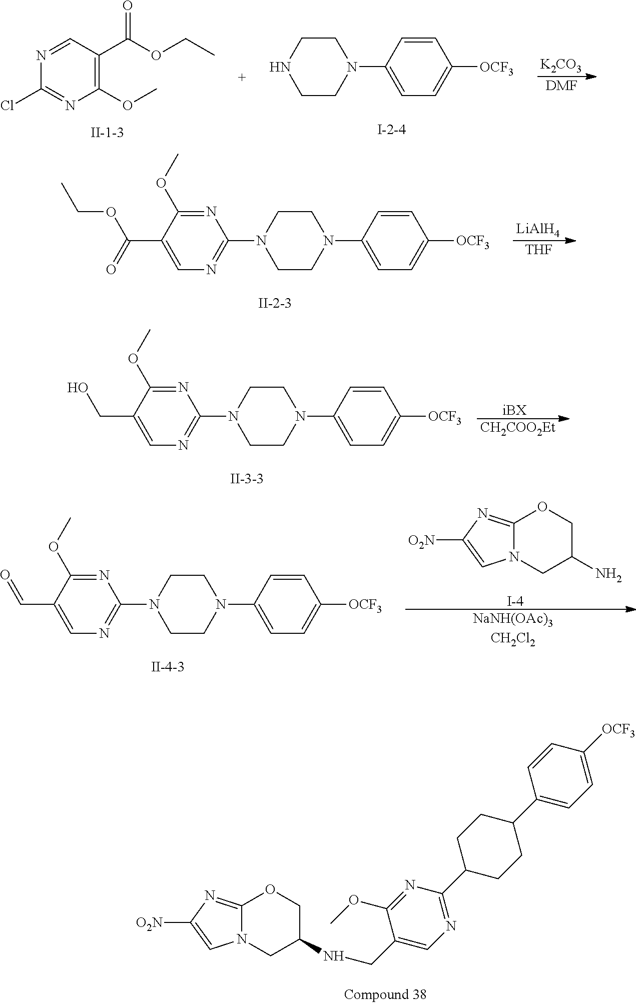

(1) 4-(4-(trifluoromethoxy)phenyl)piperazine I-2-4 (492 mg, 2.00 mmol) and ethyl 2-chloro-4-methoxypyrimidin-5-carboxylate II-1-3 (475 mg, 2.20 mmol) (reference: WO 2004060308) were used as raw materials, and the operation method was the same as the method of (1) in Example 36, giving intermediate II-2-3 (750 mg, yield: 88.1%).

Intermediate II-2-3: ¹H-NMR (400 MHz, CDCl₃) δ 8.71 (s, 1H), 7.15-7.11 (m, 2H), 6.91-6.87 (m, 2H), 4.33 (q, J=7.1 Hz, 2H), 3.97 (s, 3H), 3.78-3.72 (m, 4H), 3.27-3.24 (m, 4H), 1.43 (t, J=7.1 Hz, 3H).

(2) Intermediate II-2-3 (724 mg, 1.70 mmol) and lithium aluminum hydride (65 mg, 1.70 mmol) were used as raw materials, and the operation method was the same as the method of (2) in Example 36, giving intermediate II-3-3 (526 mg, yield: 80.7%). Intermediate II-3-3: ESI-LR: 385.14 [M+1]⁺.

(3) Intermediate II-3-3 (499 mg, 1.30 mmol) and IBX (546 mg, 1.95 mmol) were used as raw materials, and the operation method was the same as the method of (3) in Example 36, giving intermediate II-4-3 (282 mg, yield: 56.8%) as a yellow oil.

Intermediate II-4-3: ¹H-NMR (400 MHz, CDCl₃) δ 9.79 (s, 1H), 8.59 (s, 1H), 7.18-7.14 (m, 2H), 6.95-6.92 (m, 2H), 3.97 (s, 3H), 3.78-3.72 (m, 4H), 3.27-3.24 (m, 4H).

(4) Intermediate II-4-3 (271 mg, 0.71 mmol) and I-4 (131 mg, 0.71 mmol) were used as raw materials, and the operation method was the same as the method of (4) in Example 36, giving compound 38 (143 mg, yield: 36.8%) as a pale yellow powder.

Compound 38: ¹H-NMR (400 MHz, CDCl₃) δ 8.09 (s, 1H), 7.40 (s, 1H), 7.13-7.06 (m, 2H), 6.99-6.91 (m, 2H), 4.47-4.38 (m, 1H), 4.15 (dd, J=12.3, 4.4 Hz, 1H), 3.97-3.88 (m, 8H), 3.78-3.74 (m, 2H), 3.38-3.34 (m, 1H), 3.24-3.19 (m, 4H).

ESI-LR: 551.19 [M+1]⁺.

### Example 39

### (S)—N-((4-chloro-2-(4-(4-(trifluoromethoxy)phenyl)piperazin-1-yl)pyrimidin-5-yl)methyl)-2-nitro-6,7-dihydro-5H-imidazo[2,1-b][1,3]oxazin-6-amine (compound 39)

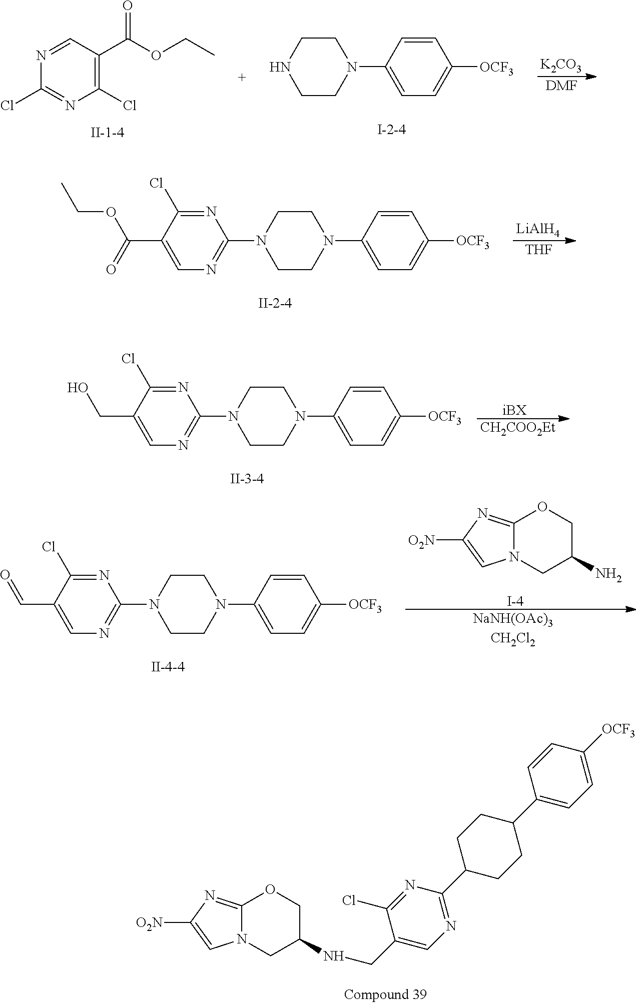

(1) 4-(4-(trifluoromethoxy)phenyl)piperazine I-2-4 (984 mg, 4.00 mmol) and ethyl 2,4-dichloro-pyrimidin-5-carboxylate II-1-4 (972 mg, 4.40 mmol) (reference: WO 2009074749) were used as raw materials, and the operation method was the same as the method of (1) in Example 36, giving intermediate II-2-4 (782 mg, yield: 45.5%).

Intermediate II-2-4: ¹H-NMR (400 MHz, CDCl₃) δ 8.75 (s, 1H), 7.15-7.11 (m, 2H), 6.91-6.87 (m, 2H), 4.33 (q, J=7.1 Hz, 2H), 3.78-3.72 (m, 4H), 3.27-3.24 (m, 4H), 1.43 (t, J=7.1 Hz, 3H).

(2) Intermediate II-2-4 (731 mg, 1.70 mmol) and lithium aluminum hydride (65 mg, 1.70 mmol) were used as raw materials, and the operation method was the same as the method of (2) in Example 36, giving intermediate II-3-4 (449 mg, yield: 68.1%). Intermediate II-3-4: ESI-LR: 389.09 [M+1]⁺.

(3) Intermediate II-3-4 (426 mg, 1.10 mmol) and IBX (462 mg, 1.65 mmol) were used as raw materials, and the operation method was the same as the method of (3) in Example 36, giving intermediate II-4-4 (257 mg, yield: 60.7%) as a yellow oil.

Intermediate II-4-4: ¹H-NMR (400 MHz, CDCl₃) δ 9.79 (s, 1H), 8.61 (s, 1H), 7.18-7.14 (m, 2H), 6.95-6.92 (m, 2H), 3.78-3.72 (m, 4H), 3.27-3.24 (m, 4H).

(4) Intermediate II-4-4 (231 mg, 0.60 mmol) and I-4 (110 mg, 0.60 mmol) were used as raw materials, and the operation method was the same as the method of (4) in Example 36, giving compound 39 (144 mg, yield: 43.5%) as a pale yellow powder.

Compound 39: ¹H-NMR (400 MHz, CDCl₃) δ 8.11 (s, 1H), 7.43 (s, 1H), 7.13-7.06 (m, 2H), 6.99-6.91 (m, 2H), 4.47-4.38 (m, 1H), 4.15 (dd, J=12.3, 4.4 Hz, 1H), 3.97-3.88 (m, 5H), 3.78-3.74 (m, 2H), 3.38-3.34 (m, 1H), 3.24-3.19 (m, 4H). ESI-LR: 555.14 [M+1]⁺.

### Example 40

### (S)-5-(((2-nitro-6,7-dihydro-5H-imidazo[2,1-b][1,3]oxazin-6-yl)amino)methyl)-2-(4-(4-(trifluoromethoxy)phenyl)piperazin-1-yl)pyrimidine-4-carbonitrile (compound 40)

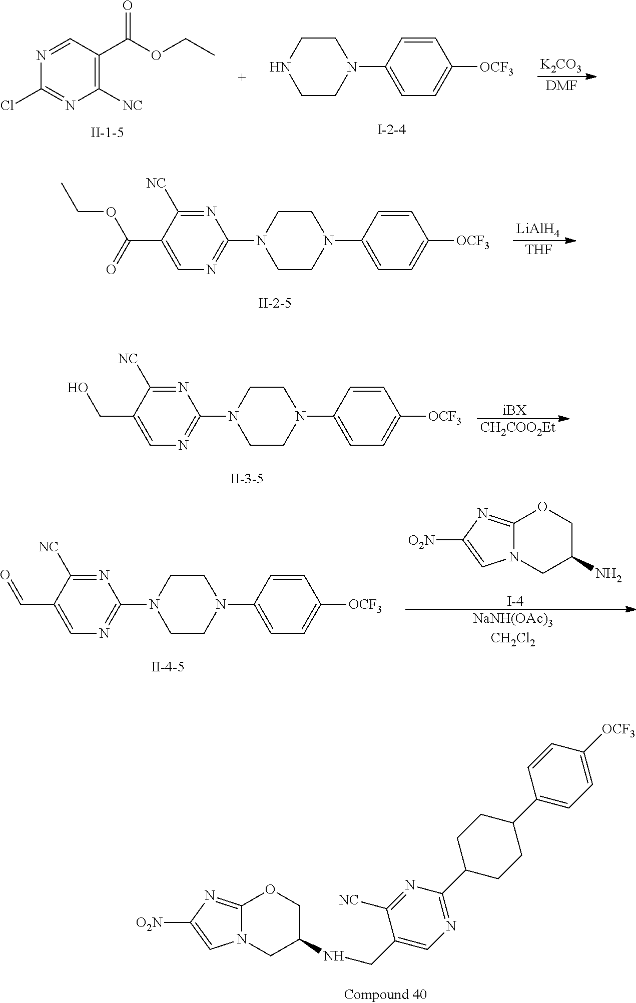

(1) 4-(4-(trifluoromethoxy)phenyl)piperazine I-2-4 (492 mg, 2.00 mmol) and ethyl 2-chloro-4-cyano pyrimidin-5-carboxylate II-1-5 (464 mg, 2.20 mmol) (reference: WO 2010036632) were used as raw materials, and the operation method was the same as the method of (1) in Example 36, giving intermediate II-2-5 (726 mg, yield: 86.3%).

Intermediate II-2-5: ¹H-NMR (400 MHz, CDCl₃) δ 8.99 (s, 1H), 7.17-7.14 (m, 2H), 6.95-6.92 (m, 2H), 4.43 (q, J=7.1 Hz, 2H), 4.12-4.09 (m, 4H), 3.27-3.24 (m, 4H), 1.43 (t, J=7.1 Hz, 3H).

(2) Intermediate II-2-5 (715 mg, 1.70 mmol) and lithium aluminum hydride (65 mg, 1.70 mmol) were used as raw materials, and the operation method was the same as the method of (2) in Example 36, giving intermediate II-3-5 (417 mg, yield: 64.8%). Intermediate II-3-5: ESI-LR: 380.13 [M+1]⁺.

(3) Intermediate II-3-5 (417 mg, 1.10 mmol) and IBX (462 mg, 1.65 mmol) were used as raw materials, and the operation method was the same as the method of (3) in Example 36, giving intermediate II-4-5 (254 mg, yield: 61.4%) as a yellow oil.

Intermediate II-4-5: ¹H-NMR (400 MHz, CDCl₃) δ 9.79 (s, 1H), 9.04 (s, 1H), 7.20-7.15 (m, 2H), 6.95-6.92 (m, 2H), 4.12-4.09 (m, 4H), 3.27-3.24 (m, 4H).

(4) Intermediate II-4-5 (226 mg, 0.60 mmol) and I-5 (110 mg, 0.60 mmol) were used as raw materials, and the operation method was the same as the method of (4) in Example 36, giving compound 40 (126 mg, yield: 38.7%) as a pale yellow powder.

Compound 40: ¹H-NMR (400 MHz, CDCl₃) δ 8.46 (s, 1H), 7.40 (s, 1H), 7.15-7.12 (m, 2H), 6.95-6.91 (m, 2H), 4.46-4.44 (m, 1H), 4.23 (dd, J=12.6, 4.4 Hz, 1H), 4.08 (dd, J=12.6, 3.6 Hz, 1H), 4.00-3.95 (m, 4H), 3.93 (s, 2H), 3.47-3.43 (m, 1H), 3.24-3.19 (m, 4H). ESI-LR: 546.17 [M+1]⁺.

### Example 41

### (S)-2-nitro-N-((2-(4-(4-(trifluoromethoxy)phenyl)piperazin-1-yl)-4-(trifluoromethyl)pyrimidin-5-yl)methyl)-6,7-dihydro-5H-imidazo[2,1-b][1,3]oxazin-6-amine (compound 41)

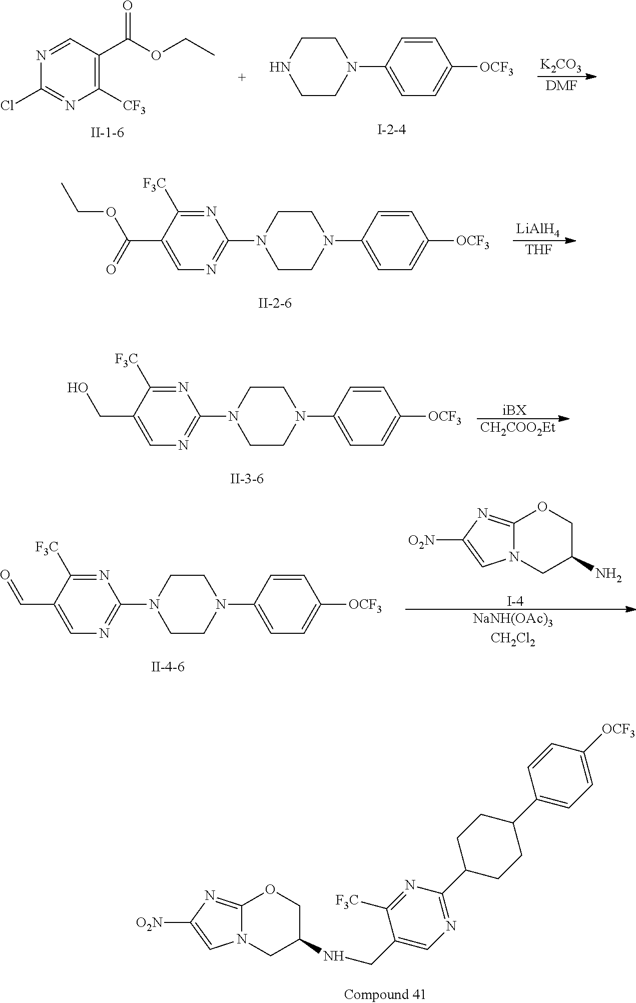

(1) 4-(4-(trifluoromethoxy)phenyl)piperazine I-2-4 (492 mg, 2.00 mmol) and ethyl 2-chloro-4-(trifluoromethyl)pyrimidin-5-carboxylate II-1-6 (558 mg, 2.20 mmol) (reference: WO 2006048297) were used as raw materials, and the operation method was the same as the method of (1) in Example 36, giving intermediate II-2-6 (790 mg, yield: 85.1%).

Intermediate II-2-6: ¹H-NMR (400 MHz, CDCl₃) δ 8.42 (s, 1H), 7.16-7.12 (m, 2H), 6.94-6.91 (m, 2H), 4.43 (q, J=7.1 Hz, 2H), 4.01-3.96 (m, 4H), 3.27-3.24 (m, 4H), 1.43 (t, J=7.1 Hz, 3H).

(2) Intermediate II-2-6 (788 mg, 1.70 mmol) and lithium aluminum hydride (65 mg, 1.70 mmol) were used as raw materials, and the operation method was the same as the method of (2) in Example 36, giving intermediate II-3-6 (483 mg, yield: 67.4%). Intermediate II-3-6: ESI-LR: 423.12 [M+1]⁺.

(3) Intermediate II-3-6 (464 mg, 1.10 mmol) and IBX (462 mg, 1.65 mmol) were used as raw materials, and the operation method was the same as the method of (3) in Example 36, giving intermediate II-4-6 (254 mg, yield: 61.4%) as a yellow oil.

Intermediate II-4-6: ¹H-NMR (400 MHz, CDCl₃) δ 9.79 (s, 1H), 8.56 (s, 1H), 7.19-7.15 (m, 2H), 6.95-6.92 (m, 2H), 4.01-3.96 (m, 4H), 3.27-3.24 (m, 4H).

(4) Intermediate II-4-6 (252 mg, 0.60 mmol) and I-4 (110 mg, 0.60 mmol) were used as raw materials, and the operation method was the same as the method of (4) in Example 36, giving compound 41 (146 mg, yield: 41.6%) as a pale yellow powder.

Compound 41: ¹H-NMR (400 MHz, CDCl₃) δ 8.51 (s, 1H), 7.38 (s, 1H), 7.14-7.11 (m, 2H), 6.93-6.90 (m, 2H), 4.46-4.44 (m, 1H), 4.36 (dd, J=12.6, 4.4 Hz, 1H), 4.18 (dd, J=12.5, 4.5 Hz, 1H), 4.02-3.98 (m, 4H), 3.89 (s, 2H), 3.47-3.43 (m, 1H), 3.24-3.19 (m, 4H). ESI-LR: 589.17 [M+1]⁺.

### Example 42

### (S)—N-((4-cyclopropyl-2-(4-(4-(trifluoromethoxy)phenyl)piperazin-1-yl)pyrimidin-5-yl)methyl)-2-nitro-6,7-dihydro-5H-imidazo[2,1-b][1,3]oxazin-6-amine (compound 42)

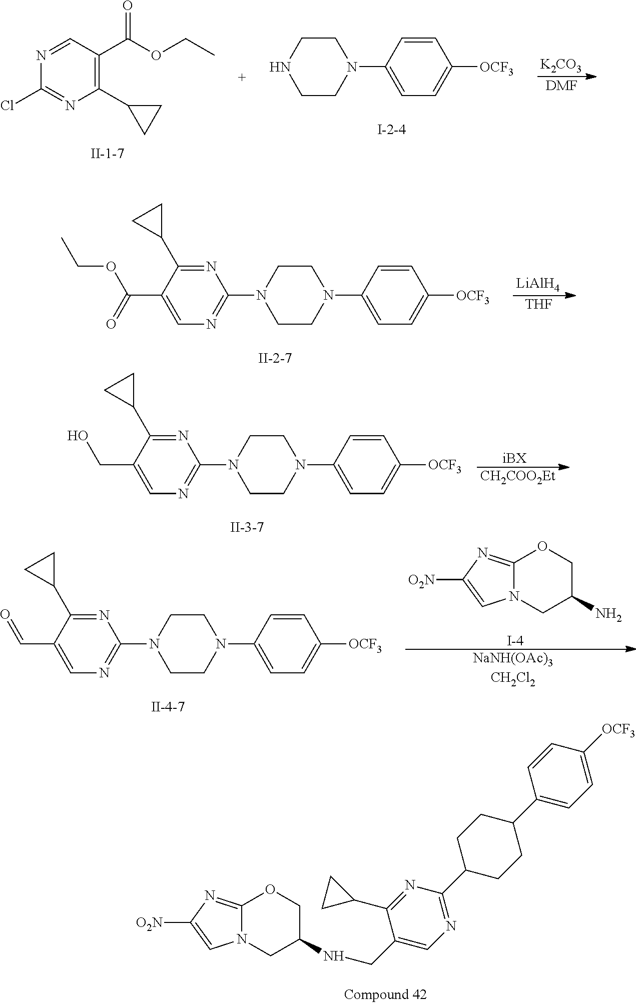

(1) 4-(4-(trifluoromethoxy)phenyl)piperazine I-2-4 (492 mg, 2.00 mmol) and ethyl 2-chloro-4-cyclopropylpyrimidin-5-carboxylate II-1-7 (497 mg, 2.20 mmol) (reference: WO 2012129338) were used as raw materials, and the operation method was the same as the method of (1) in Example 36, giving intermediate II-2-7 (751 mg, yield: 86.2%).

Intermediate II-2-7: ¹H-NMR (400 MHz, CDCl₃) δ 8.57 (s, 1H), 7.18-7.14 (m, 2H), 6.95-6.92 (m, 2H), 4.43 (q, J=7.1 Hz, 2H), 4.10-4.07 (m, 4H), 3.27-3.24 (m, 4H), 2.25-2.20 (m, 1H), 1.43 (t, J=7.1 Hz, 3H), 1.28-1.26 (m, 2H), 1.10-1.04 (m, 2H).

(2) Intermediate II-2-7 (741 mg, 1.70 mmol) and lithium aluminum hydride (65 mg, 1.70 mmol) were used as raw materials, and the operation method was the same as the method of (2) in Example 36, giving intermediate II-3-7 (505 mg, yield: 75.4%). Intermediate II-3-7: ESI-LR: 395.16 [M+1]⁺.

(3) Intermediate II-3-7 (433 mg, 1.10 mmol) and IBX (462 mg, 1.65 mmol) were used as raw materials, and the operation method was the same as the method of (3) in Example 36, giving intermediate II-4-7 (223 mg, yield: 51.8%) as a yellow oil.

Intermediate II-4-7: ¹H-NMR (400 MHz, CDCl₃) δ 9.79 (s, 1H), 8.59 (s, 1H), 7.18-7.14 (m, 2H), 6.95-6.92 (m, 2H), 4.16-4.13 (m, 4H), 3.27-3.24 (m, 4H), 2.25-2.20 (m, 1H), 1.28-1.26 (m, 2H), 1.10-1.04 (m, 2H).

(4) Intermediate II-4-7 (196 mg, 0.50 mmol) and I-4 (92 mg, 0.50 mmol) were used as raw materials, and the operation method was the same as the method of (4) in Example 36, giving compound 42 (107 mg, yield: 38.4%) as a pale yellow powder.

Compound 42: ¹H-NMR (400 MHz, CDCl₃) δ 8.13 (s, 1H), 7.74 (s, 1H), 7.18-7.09 (m, 2H), 7.05-6.94 (m, 2H), 4.55-4.44 (m, 2H), 4.26 (dd, J=12.7, 4.1 Hz, 1H), 4.07 (dd, J=12.8, 4.0 Hz, 1H), 3.97-3.88 (m, 4H), 3.78-3.74 (m, 2H), 3.43-3.40 (m, 1H), 3.26-3.14 (m, 4H), 2.30-2.25 (m, 1H), 1.34-1.29 (m, 2H), 1.15-1.09 (m, 2H). ESI-LR: 561.21 [M+1]⁺.

### Example 43

### (S)—N-((4,6-dimethyl-2-(4-(4-(trifluoromethoxy)phenyl)piperazin-1-yl)pyrimidin-5-yl)methyl)-2-nitro-6,7-dihydro-5H-imidazo[2,1-b][1,3]oxazin-6-amine (compound 43)

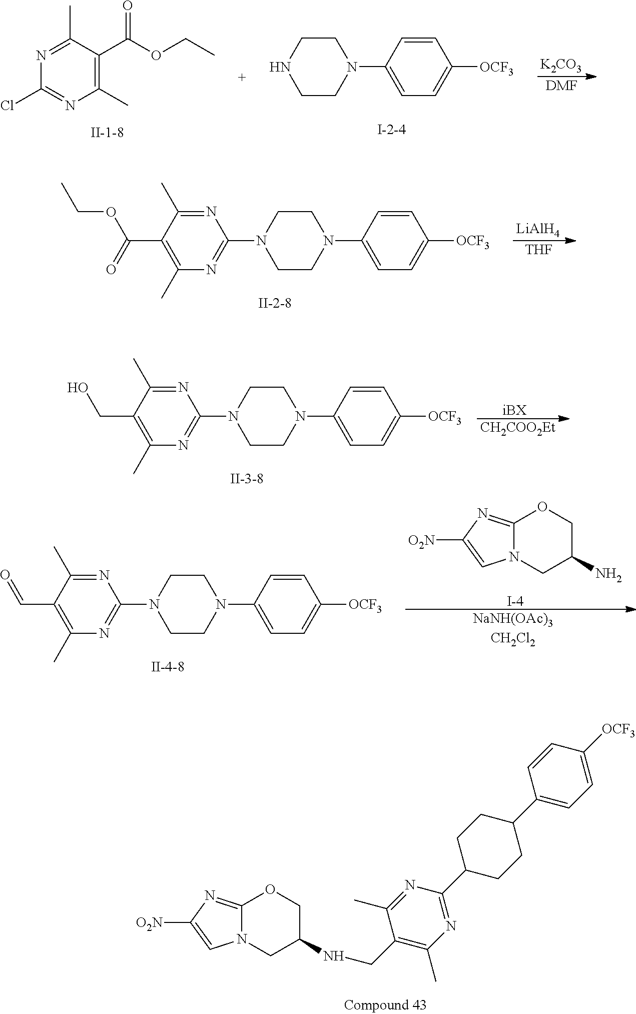

(1) 4-(4-(trifluoromethoxy)phenyl)piperazine I-2-4 (492 mg, 2.00 mmol) and ethyl 2-chloro-4,6-dimethylpyrimidin-5-carboxylate II-1-8 (470 mg, 2.20 mmol) (reference: WO 2008157404) were used as raw materials, and the operation method was the same as the method of (1) in Example 36, giving intermediate II-2-8 (832 mg, yield: 89.3%).

Intermediate II-2-8: ¹H-NMR (400 MHz, CDCl₃) δ 7.18-7.14 (m, 2H), 6.95-6.92 (m, 2H), 4.43 (q, J=7.1 Hz, 2H), 4.10-4.07 (m, 4H), 3.27-3.24 (m, 4H), 2.33 (s, 6H), 1.43 (t, J=7.1 Hz, 3H).

(2) Intermediate II-2-8 (697 mg, 1.70 mmol) and lithium aluminum hydride (65 mg, 1.70 mmol) were used as raw materials, and the operation method was the same as the method of (2) in Example 36, giving intermediate II-3-8 (438 mg, yield: 67.5%). Intermediate II-3-8: ESI-LR: 383.16 [M+1]⁺.

(3) Intermediate II-3-8 (420 mg, 1.10 mmol) and IBX (462 mg, 1.65 mmol) were used as raw materials, and the operation method was the same as the method of (3) in Example 36, giving intermediate II-4-8 (203 mg, yield: 48.7%) as a yellow oil.

Intermediate II-4-8: ¹H-NMR (400 MHz, CDCl₃) δ 9.79 (s, 1H), 7.18-7.14 (m, 2H), 6.95-6.92 (m, 2H), 4.16-4.13 (m, 4H), 3.27-3.24 (m, 4H), 2.38 (s, 6H).

(4) Intermediate II-4-8 (190 mg, 0.50 mmol) and I-4 (92 mg, 0.50 mmol) were used as raw materials, and the operation method was the same as the method of (4) in Example 36, giving compound 43 (73 mg, yield: 26.8%) as a pale yellow powder.

Compound 43: ¹H-NMR (400 MHz, CDCl₃) δ 7.48 (s, 1H), 7.18-7.09 (m, 2H), 7.05-6.94 (m, 2H), 4.50-4.43 (m, 2H), 4.22 (dd, J=12.7, 4.1 Hz, 1H), 3.95-3.88 (m, 5H), 3.86-3.75 (m, 2H), 3.46 (s, 1H), 3.22-3.18 (m, 4H), 2.38 (s, 6H). ESI-LR: 549.21 [M+1]⁺.

### Example 44

### (S)—N-methyl-2-nitro-N-((2-(4-(4-(trifluoromethoxy)phenyl)piperazin-1-yl)pyrimidin-5-yl)methyl)-6,7-dihydro-5H-imidazo[2,1-b][1,3]oxazin-6-amine (compound 44)

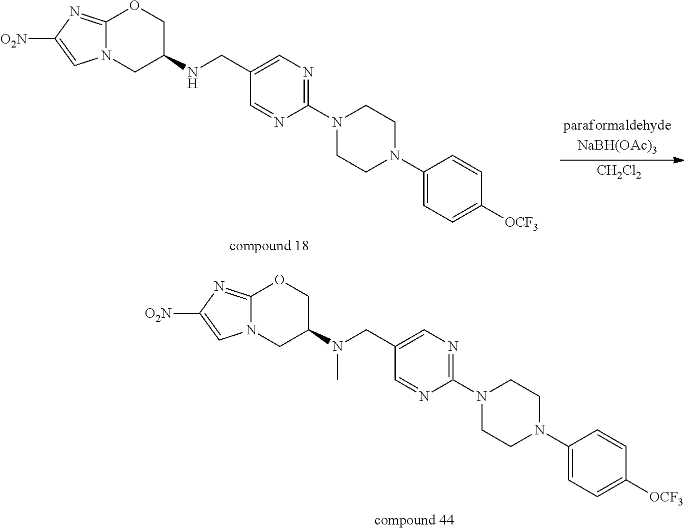 [SMILES: O=[N+]([O-])c1cn2c(n1)OC[C@@H](NCc1cnc(N3CCN(c4ccc(OC(F)(F)F)cc4)CC3)nc1)C2 ; "CH2Cl2" ; CN(Cc1cnc(N2CCN(c3ccc(OC(F)(F)F)cc3)CC2)nc1)[C@@H]1COc2nc([N+](=O)[O-])cn2C1]

Compound 18 (104 mg, 0.20 mmol) was dissolved in tetrahydrofuran (10 mL), then raw material paraformaldehyde (60 mg) and 3 drops of acetic acid in a catalytic amount were added to the solution, the mixture was reacted at room temperature overnight, NaBH(OAc)₃ (168 mg, 0.8 mmol) was added thereto, and the reaction was continued at room temperature overnight. A solution of sodium bicarbonate (10 mL) was added, the layers were separated, the aqueous layer was extracted with dichloromethane (20 mL*2), the dichloromethane layers were combined, washed with saturated sodium chloride solution, dried over anhydrous sodium sulfate and spin dried, and the residue was purified by column chromatography (dichloromethane:methanol=100:1), giving compound 44 (71 mg, yield: 67.3%) as a pale yellow powder.

Compound 44: ¹H-NMR (400 MHz, CDCl₃) δ 8.22 (s, 2H), 7.41 (s, 1H), 7.13 (d, J=8.5 Hz, 2H), 6.93 (d, J=8.9 Hz, 2H), 4.52-4.46 (m, 2H), 4.16 (dd, J=12.3, 4.5 Hz, 1H), 3.99-3.94 (m, 4H), 3.59-3.54 (m, 2H), 3.33 (s, 1H), 3.26-3.18 (m, 4H), 2.32 (s, 3H). ESI-LR: 535.20 [M+1]⁺.

### Example 45

### (S)—N-ethyl-2-nitro-N-((2-(4-(4-(trifluoromethoxy)phenyl)piperazin-1-yl)pyrimidin-5-yl)methyl)-6,7-dihydro-5H-imidazo[2,1-b][1,3]oxazin-6-amine (compound 45)

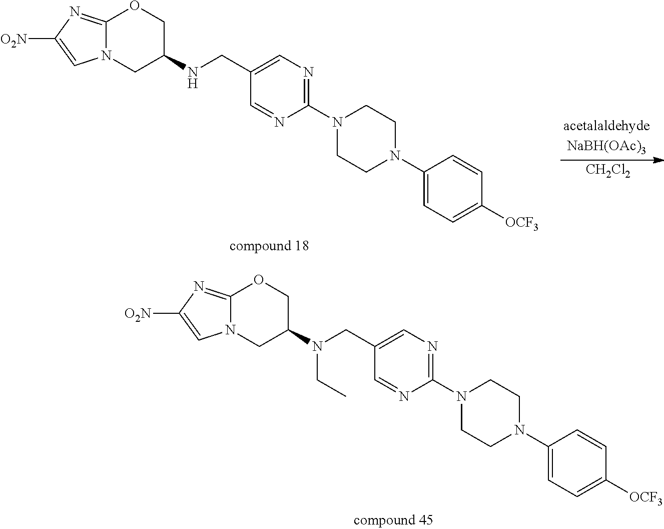 [SMILES: O=[N+]([O-])c1cn2c(n1)OC[C@@H](NCc1cnc(N3CCN(c4ccc(OC(F)(F)F)cc4)CC3)nc1)C2 ; "CH2Cl2" ; CCN(Cc1cnc(N2CCN(c3ccc(OC(F)(F)F)cc3)CC2)nc1)[C@@H]1COc2nc([N+](=O)[O-])cn2C1]

Compound 18 (104 mg, 0.20 mmol) and acetaldehyde (18 mg) were used as raw materials, and the operation method was the same as the method of Example 44, giving compound 45 (79 mg, yield: 72.3%) as a pale yellow powder.

Compound 45: ¹H-NMR (400 MHz, CDCl₃) δ 8.22 (s, 2H), 7.41 (s, 1H), 7.13 (d, J=8.5 Hz, 2H), 6.93 (d, J=8.9 Hz, 2H), 4.52-4.46 (m, 2H), 4.16 (dd, J=12.3, 4.5 Hz, 1H), 3.99-3.94 (m, 4H), 3.59-3.54 (m, 2H), 3.33 (s, 1H), 3.26-3.18 (m, 4H), 2.71 (q, J=7.1 Hz, 2H), 1.09 (t, J=7.1 Hz, 3H). ESI-LR: 549.21 [M+1]⁺.

### Example 46

### (S)-2-nitro-N-(2-(6-(4-(4-(trifluoromethoxy)phenyl)piperazin-1-yl)pyrid-3-yl)ethyl)-6,7-dihydro-5H-imidazo[2,1-b][1,3]oxazin-6-amine (compound 46)

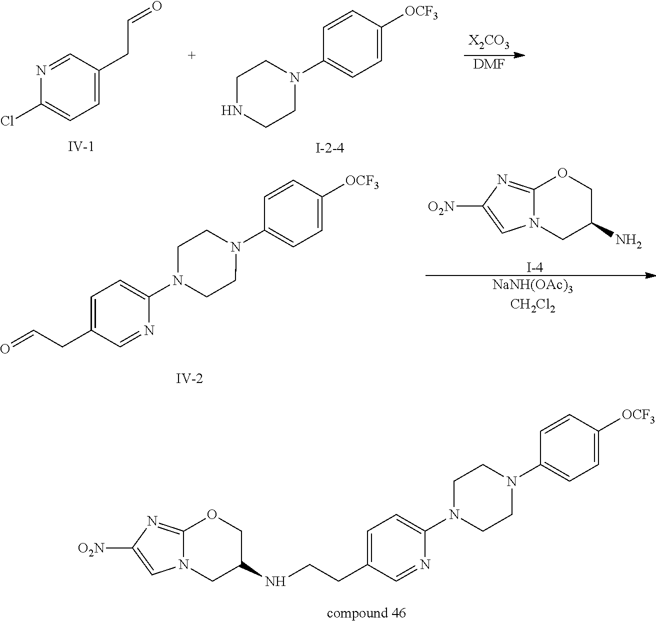

(1) 4-(4-(trifluoromethoxy)phenyl)piperazine I-2-4 (492 mg, 2.00 mmol) and 2-(6-chloro-pyridin-3-yl)acetaldehyde IV-1 (341 mg, 2.20 mmol) were dissolved in DMF (8 mL), K₂CO₃ (828 mg, 6.00 mmol) was added to the solution dropwise and the mixture was reacted for 6 hours at 90° C. after the dropwise addition was completed. The reaction was completely cooled to room temperature, poured into ice water, extracted with ethyl acetate (20 mL*2), dried over anhydrous sodium sulfate, filtered, spin dried and purified by column chromatography (petroleum ether:ethyl acetate=4:1), giving intermediate IV-2 (638 mg, yield: 87.5%) as a pale yellow solid.

Intermediate IV-2: ¹H-NMR (400 MHz, CDCl₃) δ 9.78 (s, 1H), 8.57-8.53 (m, 1H), 7.93 (dd, J=9.1, 2.3 Hz, 1H), 7.18-7.12 (m, 2H), 6.95-6.88 (m, 2H), 6.70 (d, J=9.1 Hz, 1H), 4.95-4.31 (m, 4H), 3.66 (d, J=1.2, 2H), 3.37-3.32 (m, 4H).

(2) Intermediate IV-2 (259 mg, 0.71 mmol) and triethylamine (93 mg, 0.92 mmol) were dissolved in dichloromethane (10 mL), then raw material I-4 (131 mg, 0.71 mmol) was added to the solution, the mixture was reacted at room temperature overnight, NaBH(OAc)₃ (602 mg, 2.84 mmol) was added thereto, and the reaction was continued at room temperature overnight. A solution of sodium bicarbonate (10 mL) was added, the layers were separated, the aqueous layer was extracted with dichloromethane (20 mL*2), the dichloromethane layers were combined, washed with saturated sodium chloride solution, dried over anhydrous sodium sulfate and spin dried, and the residue was purified by column chromatography (dichloromethane:methanol=50:1), giving compound 46 (265 mg, yield: 70.2%) as a pale yellow powder.

Compound 46: ¹H-NMR (400 MHz, CDCl₃) δ 8.11 (s, 1H), 7.48 (dd, J=8.6, 2.4 Hz, 1H), 7.36 (s, 1H), 7.13 (d, J=8.7 Hz, 2H), 6.94 (t, J=6.3 Hz, 2H), 6.69 (d, J=8.7 Hz, 1H), 4.41-4.35 (m, 2H), 4.14 (dd, J=12.3, 4.5 Hz, 1H), 3.92 (dd, J=12.2, 3.4 Hz, 1H), 3.79-3.70 (m, 4H), 3.40 (dd, J=4.7, 2.6 Hz, 1H), 3.31-3.25 (m, 4H) 2.91-3.86 (m, 2H), 2.78-3.74 (t, J=7.3 Hz, 2H). ESI-LR: 534.20 [M+1]⁺.

### Example 47

### (S)-2-nitro-N-((6-(4-(4-(trifluoromethoxy)phenyl)piperazin-1-yl)pyrid-3-yl)methyl)-6,7-dihydro-5H-imidazo[2,1-b][1,3]oxazin-6-amine phosphate (compound 47)

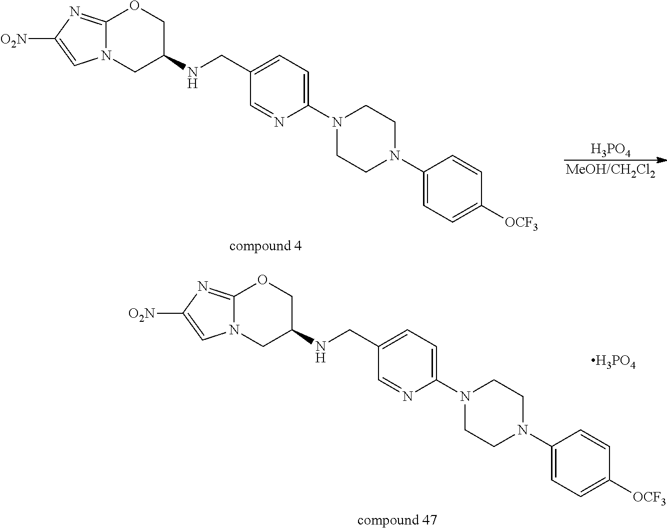 [SMILES: O=[N+]([O-])c1cn2c(n1)OC[C@@H](NCc1ccc(N3CCN(c4ccc(OC(F)(F)F)cc4)CC3)nc1)C2 ; "H3PO4" ; "MeOH/CH2Cl2" ; O=[N+]([O-])c1cn2c(n1)OC[C@@H](NCc1ccc(N3CCN(c4ccc(OC(F)(F)F)cc4)CC3)nc1)C2 ; "·H3PO4"]

Compound 4 (1.04 g, 2.0 mmol) was dissolved in the mixed solvent of dichloromethane (10 mL) and methanol (6 mL), phosphoric acid (253 mg, 2.2 mmol) was added dropwise after the compound was completely dissolved, and the solution was heated to reflux. After cooling down, a solid was precipitated, filtered and dried, giving compound 47 (839 mg, 69.8%) as a white solid, melting point: 181° C.-183° C.

Compound 47: the elemental analysis thereof: C23H27F3N7O8P, theoretical values of the following elements: C, 44.74; H, 4.41; and N, 15.88; and measured values of the following elements: C, 44.68; H, 4.43; and N, 15.81.

### Examples 48-50: Preparation of Compounds 48-50

Similar to the synthesis of compound 47, compounds 48-50 of Table 1 can be prepared according to the procedure of Example 47, and the acids used specifically and the salt melting points and yields of the resulting compounds are shown in Table 2.

TABLE 2

| | Compound | | Salt melting point | |
|---|---|---|---|---|
| Example | No. | Acid | (° C.) | Yield |
| Example | 48 | hydrochloric acid | 192-194 | 54.2% |
| 48 | | | | |
| Example | 49 | methanesulfonic | 175-177 | 70.2% |
| 49 | | acid | | |
| Example | 50 | fumaric acid | 143-145 | 80.7% |
| 50 | | | | |

### Example 51 Activity Test for Mycobacterium tuberculosis

The tested strain H37Rv was transferred to liquid medium and cultured for 2 weeks at 37° C.; a small amount of the cultured bacterial solution was pipetted and placed in 4 mL of liquid medium; 10-20 sterile glass beads with a diameter of 2-3 mm were added; the mixture was shaken for 20-30 s and left to sediment for 10-20 min; the supernatant of the bacterial suspension was pipetted and adjusted to a turbidity of 1 MCF (equivalent to 1×10⁷ CFU/mL) with the liquid medium for use. Each drug was dissolved to 1 mg/mL with an appropriate amount of DMSO and filtered with a 0.22 μm filter. Then, the solution was diluted to a desired experimental concentration with the liquid medium. The final concentrations of the tested drugs were set as follows: 0.001 μg/mL, 0.002 μg/mL, 0.0039 μg/mL, 0.0078 μg/mL, 0.0165 μg/mL, 0.03125 μg/mL, 0.0625 μg/mL, 0.125 μg/mL, 0.25 μg/mL, 0.5 μg/mL and 1 μg/mL, a total of 11 concentration gradients. 100 μL of each of the above-mentioned drug solutions was added to a 96-well microwell plate, then 100 μL bacterial solution with a concentration of 1 mg/mL was added to allow the drug concentration to reach the final set concentration, and cultured at 37° C. Three groups in parallel were set for each drug dilution with inoculation amounts of 100%, 10% and 1%, respectively, while no drug was added to the control group. The minimum inhibitory concentration (MIC) of each drug against Mycobacterium tuberculosis was observed and compared to the MIC results of the first-line anti-tuberculosis drug ethambutol and PA-824 which is in the clinical study stage. The results are shown in Table 3 below.

TABLE 3
MIC values of some compounds against
Mycobacterium tuberculosis H37Rv

| | Minimum inhibitory |
|---|---|
| | concentration against |
| Compound | H37Rv (μg/mL) |
| Compound 1 | 0.0078 |
| Compound 3 | 0.0156 |
| Compound 4 | 0.00195 |
| Compound 5 | 0.0039 |
| Compound 6 | 0.00195 |
| Compound 10 | 0.00195 |
| Compound 11 | 0.03125 |
| Compound 13 | 0.0156 |
| Compound 14 | 0.0078 |
| Compound 15 | 0.03125 |
| Compound 18 | 0.0078 |
| Compound 19 | 0.0078 |
| Compound 20 | 0.00195 |
| Compound 24 | 0.0039 |
| Compound 25 | 0.0156 |
| Compound 28 | 0.0156 |
| Compound 31 | 0.0078 |
| Compound 36 | 0.0078 |
| Compound 38 | 0.0156 |
| Compound 40 | 0.0078 |
| Compound 41 | 0.03125 |
| Compound 44 | 0.00195 |
| Ethambutol | 0.5 |
| PA-824 | 0.0625 |

As shown in Table 3, in vitro screening results for H37Rv showed that compound 4, compound 6, compound 10, compound 20 and compound 44 were the most active, the minimum inhibitory concentration (MIC) against H37Rv of which was 256 times of that of ethambutol and 32 times of the activity of PA-824 which is in clinical study; and compound 5 and compound 24 showed the same strong anti Mycobacterium tuberculosis activity, which was 128 times of that of ethambutol and 16 times of that of PA-824, respectively. Compound 1, compound 14, compound 18, compound 19, compound 31, compound 36 and compound 40 showed the same intensity of activity, the anti Mycobacterium tuberculosis activity of which was 64 times of that of ethambutol and 8 times of that of PA-824, respectively.

These results indicate that the compounds of the present invention have much higher anti Mycobacterium tuberculosis activity than the first-line anti-tuberculosis drug ethambutol and PA-824 which is in the clinical study stage.

### Example 52 Test for Drug-Resistant Tuberculosis

Tested strains (246: streptomycin-resistant; 242: isoniazid-resistant; and 261: rifampicin-resistant; Mycobacterium tuberculosis clinical isolates were clinically isolated from Shanghai Pulmonary Hospital, with steps as follows: a. collecting sputum specimens from inpatients at Department of Tuberculosis, Shanghai Pulmonary Hospital, inoculating the sputum specimens to a modified Roche medium after alkali treatment and culturing for 2 weeks; and b. measuring drug sensitivity with the absolute concentration method: scraping fresh cultures from the medium slant, adjusting the bacterial solution with physiological saline to a turbidity of 1 MCF (1 mg/mL), diluting to 10-2 mg/mL, inoculating 0.1 mL to a drug sensitive medium and observing the results after four weeks; reference material: Tuberculosis Diagnosis Laboratory Inspection Specification, edited by Chinese Anti-tuberculosis Association basic Professional Committee, China Education and Culture Press, January 2006) were transferred to a liquid medium and cultured for 2 weeks at 37° C.; a small amount of the cultured bacterial solution was pipetted and placed in 4 mL of liquid medium; 10-20 sterile glass beads with a diameter of 2-3 mm were added; the mixture was shaken for 20-30 s and left to sediment for 10-20 min; the supernatant of the bacterial suspension was pipetted and adjusted to a turbidity of 1 MCF (equivalent to 1×10⁷ CFU/mL) with the liquid medium for use. Each drug was dissolved to 1 mg/mL with an appropriate amount of DMSO and filtered with a 0.22 μm filter. Then, the solution was diluted to a desired experimental concentration with the liquid medium. The final concentrations of the tested drugs were set as follows: 0.0039 μg/mL, 0.0078 μg/mL, 0.0165 μg/mL, 0.03125 μg/mL, 0.0625 μg/mL, 0.125 μg/mL, 0.25 μg/mL, 0.5 μg/mL, 1 μg/mL, 2 μg/mL and 4 μg/mL, a total of 11 concentration gradients. 100 μL of each of the above-mentioned drug solutions was added to a 96-well microwell plate, then 100 μL bacterial solution with a concentration of 1 mg/mL was added to allow the drug concentration to reach the final set concentration, and cultured at 37° C. Three groups in parallel were set for each drug dilution with inoculation amounts of 100%, 10% and 1%, respectively, while no drug was added to the control group. The minimum inhibitory concentration (MIC) of each drug against Mycobacterium tuberculosis was observed and compared to the MIC result of PA-824. The results are shown in the table below.

TABLE 4
MIC values of some compounds against drug-
resistant Mycobacterium tuberculosis

| | MIC (μg/mL) | | |
|---|---|---|---|
| Drug-resistant | 246 | 242 | 261 |
| bacterium | (S single- | (H single- | (R single- |
| Compound | resistant) | resistant) | resistant) |
| Compound 1 | 0.0078 | 0.0078 | 0.0078 |
| Compound 4 | 0.00195 | 0.00195 | 0.00195 |
| Compound 5 | 0.00195 | 0.0039 | 0.00195 |
| Compound 6 | 0.0078 | 0.0156 | 0.0078 |
| Compound 10 | 0.00195 | 0.00195 | 0.00195 |
| Compound 14 | 0.0078 | 0.0156 | 0.0078 |
| Compound 18 | 0.0078 | 0.0078 | 0.0078 |
| Compound 19 | 0.0078 | 0.0078 | 0.0078 |
| Compound 20 | 0.00195 | 0.00195 | 0.00195 |
| Compound 24 | 0.0039 | 0.0039 | 0.0039 |
| Compound 31 | 0.0078 | 0.0078 | 0.0078 |
| Compound 36 | 0.0078 | 0.0078 | 0.0078 |
| Compound 40 | 0.0078 | 0.0078 | 0.0078 |
| Compound 44 | 0.00195 | 0.00195 | 0.00195 |
| PA-824 | 0.5 | 1 | 0.5 |

S: streptomycin, H: isoniazid, R: rifampicin.

It can be seen from the test results in Table 4 above that all the tested compounds had a very strong activity against drug-resistant Mycobacterium tuberculosis; in particular, the MIC value of compound 4, compound 10, compound 20 and compound 44 against each drug-resistant Mycobacterium tuberculosis was 0.00195 μg/mL, which was 256, 512 and 256 times of that of the control drug PA-824, respectively; the MIC value of compound 24 against each drug-resistant Mycobacterium tuberculosis was 0.0039 μg/mL, which was 128, 256 and 128 times of that of the control drug PA-824, respectively; and the MIC value of compound 1, compound 18, compound 19, compound 36 and compound 40 against each drug-resistant Mycobacterium tuberculosis was 0.0078 μg/mL, which was 64, 128 and 64 times of that of the control drug PA-824, respectively.

The above-mentioned results indicate that the compounds of the present invention are highly active against tested drug-resistant Mycobacterium tuberculosis and the activities thereof are far superior to that of the positive control PA-824.

### Example 53 Solubility Test in Water

3-5 mg of compound to be tested was added to 0.5 mL of aqueous HCl solution (pH=1.2) and the mixture was shaken for three days on a shaker; the sample was centrifuged for 5 min at 10,000 rpm in a centrifuge; a volumetric flask (50 mL) was loaded with 2 mL of supernatant and water was added to a volume at the graduation mark to prepare a sample solution; and 2.6 mg of sample was precisely weighed into a volumetric flask (50 mL), an appropriate amount of methanol was added to dissolve the sample, and water was added to a volume at the graduation mark and shaken well to give a control sample solution. 20 μL of sample solution and control sample solution were each injected, and tested by liquid chromatography. The solubility was calculated as follows:

Solubility (mg/mL)=C (control)*25*A (sample)/A (control)

C (control): concentration of the control sample

A (sample): peak area of the liquid chromatogram of the sample solution

A (control): peak area of the liquid chromatogram of the control sample solution

TABLE 5
Water solubility of some compounds

| Compound to be tested | Solubility |
|---|---|
| Compound 1 | 0.7842 mg/mL |
| Compound 4 | 1.2572 mg/mL |
| Compound 10 | 0.5217 mg/mL |
| Compound 18 | 1.5321 mg/mL |
| Compound 19 | 1.3218 mg/mL |
| Compound 20 | 1.0238 mg/mL |
| Compound 24 | 0.7815 mg/mL |
| Compound 31 | 1.3548 mg/mL |
| Compound 36 | 1.1237 mg/mL |
| PA-824 | 0.017 mg/mL |

It can be seen from the test results in Table 5 above that all the compounds of the present invention have a good water solubility, wherein the water solubility of compound 4, compound 18, compound 19, compound 20, compound 31 and compound 36 is greater than 1 mg/mL, which is far greater than the solubility of the control PA-824.

Good water solubility can improve the pharmacokinetic properties of a drug and facilitate the preparation of pharmaceutical preparations.

### Example 54 Drug Metabolism Test

18 healthy male ICR mice with a body weight of 18-22 g were administered drugs by intragastric administration, with an administration dose of 10 mg/kg and an administration volume of 10 mL/kg, respectively. These mice were fasted for 12 h before the test and had free access to drinking water. These mice were fed 2 h after administration uniformly. 0.3 mL of blood was taken from the postocular venous plexus of a mouse at the set time points, placed in a heparinized test tube and centrifuged for 10 min at 3000 rpm; and plasma was separated and frozen in a refrigerator at −20° C. When measured, the sample was treated through the method for treating the plasma sample, and the drug concentration in plasma was determined by LC-MS/MS and the pharmacokinetic parameters of the drug were calculated.

TABLE 6
Pharmacokinetic parameters of some compounds when
orally administrated to the mice (10 mg/kg)

| | Cₘₐₓ | Tₘₐₓ | t_{1/2} | AUC₀₋ₜ | AUC_{0-∞} | MRT |
|---|---|---|---|---|---|---|
| Compound | (ng/mL) | (h) | (h) | (ng · h/L) | (ng · h/L) | (h) |
| Compound 1 | 2672 | 2.33 | 4.76 | 31322 | 32260 | 6.64 |
| Compound 4 | 1775 | 2.00 | 3.38 | 17161 | 17292 | 5.56 |
| Compound 10 | 2032 | 2.18 | 3.35 | 28751 | 28832 | 4.68 |
| Compound 18 | 1467 | 2.00 | 5.29 | 16021 | 16697 | 7.11 |
| Compound 19 | 1782 | 1.98 | 3.17 | 16278 | 16781 | 5.02 |
| Compound 20 | 2100 | 2.33 | 2.98 | 21502 | 21571 | 4.82 |
| Compound 24 | 1985 | 2.17 | 3.52 | 18204 | 18291 | 4.45 |
| Compound 31 | 2135 | 1.97 | 3.05 | 22384 | 22451 | 4.18 |
| Compound 36 | 2015 | 1.87 | 2.87 | 16078 | 16713 | 4.71 |

It can be seen from the data in Table 6 above that all the above-mentioned compounds have good pharmacokinetic properties; in particular, compound 1, compound 10, compound 20 and compound 31 showed excellent in the pharmacokinetic properties.

These indicate that the compounds of the present invention have a good druggability and are likely to be developed into effective drugs for treatment of tuberculosis.

### Example 55: Test for the Inhibitory Effect of Compounds on hERG Potassium Ion Channel

hERG potassium channel currents were recorded with the whole cell patch clamp technique at room temperature in HEK-293 cells (Creacell™, France) expressing hERG stably. A glass microelectrode with a tip resistance of about 1-4 MΩ was connected to the Axon 200A patch clamp amplifier. Clamp voltage and data record were controlled by a computer via the Axon DigiData 1322A A/D converter with the clampex 9.2 software; the cells were clamped at −80 mV; and the step voltage for inducing the hERG potassium current (I_{hERG}) was changed from −80 mV to +20 mV by providing a 2 s depolarization voltage, repolarized to −40 mV and returned to −80 mV after 4 s. This voltage step was given respectively before and after administration to induce the hERG potassium current.

Data analysis and processing were performed with the PatchMaster, GraphPad Prism 5 and Excel softwares. The degree of inhibition of different compound concentrations on the hERG potassium current (hERG tail current peak induced at −50 mV) was calculated using the following formula:

Fractional block %=[1−(I/Io)]×100%

in the formula, Fractional block represents the percent inhibition of a compound on the hERG potassium current, and I and Io represent the magnitudes of the hERG potassium current before and after dosing, respectively.

The IC₅₀ of a compound was calculated using the following equation by fitting:

I/Io=1/{1+([C]/IC₅₀)̂n}

in the equation, I and Io represent the magnitudes of the hERG potassium current before and after dosing, respectively; [C] is the compound concentration, and n is the Hill coefficient.

TABLE 7
Inhibition of some compounds on hERG:

| Compound | IC₅₀ (μm) |
|---|---|
| Compound 18 | 41.07 |
| Compound 19 | 38.28 |
| Compound 31 | 39.53 |
| PA-824 | 5.8 |

Table 7 shows that the compounds of the present invention have a weak inhibition on the hERG potassium current, suggesting that the compounds of the present invention are of good safety to the cardiovascular system and superior to the control drug PA-824 in safety.

### Example 56: Tablets

| | | |
|---|---|---|
| Tablet: active ingredient (compound 18) | 50 | g |
| Lactose | 200 | g |
| Starch | 400 | g |
| Magnesium stearate | 10 | g |

The preparation method was as follows: the above-mentioned active ingredient, lactose and starch were mixed and uniformly moistened with water; the wetted mixture was sieved and dried, sieved again and magnesium stearate were added; and then the mixture was compressed to tablets, each weighing 660 mg with the content of the active ingredient being 50 mg.

### Example 57: Capsules

| | | |
|---|---|---|
| Tablet: active ingredient (compound 18) | 50 | g |
| Starch | 400 | g |
| Microcrystalline cellulose | 200 | g |

The preparation method was as follows: the above-mentioned active ingredient, starch and microcrystalline cellulose were mixed and sieved; the mixture was homogeneously mixed in a suitable container; and the resulting mixture was loaded into hard gelatin capsules, each weighing 650 mg with the content of the active ingredient being 50 mg.

The examples described herein are for illustrative purposes only, and various modifications or changes that may be made by a skilled person should also be included in the spirit and scope of the patent application and within the scope of the appended claims.

1. A nitroimidazole compound according to formula (I) or optical isomers or pharmaceutically acceptable salts thereof: 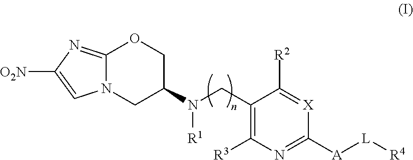
wherein, n represents an integer between 1 and 4;
L is O, S, NH or a chemical bond;
X is C or N;
R¹ is hydrogen or C₁₋₆ alkyl;
R² and R³ are the same or different and independently selected from hydrogen, halogen, cyano, trifluoromethyl, C₁₋₄ alkyl, C₃₋₆ cycloalkyl or C₁₋₄ alkoxy, respectively;
R⁴ is an aromatic ring or a heteroaromatic ring containing at least one heteroatom selected from N, O or S, wherein the aromatic ring or heteroaromatic ring is unsubstituted or substituted optionally by one to three groups independently selected from cyano, CF₃, OCF₃, halogen, methyl or methoxy;
A is selected from saturated or unsaturated C₅₋₇ cycloalkyl, C₈₋₁₀ fusedcycloalkyl, C₇₋₉ bridgedcycloalkyl or C₇₋₁₁ spirocycloalkyl, wherein at least one carbon atom of the cycloalkyl is substituted by a nitrogen atom and is linked to the heteroaromatic ring via the nitrogen atom and wherein the above-mentioned cycloalkyl is substituted by one or more fluoro, cyano, hydroxyl, C₁₋₄ alkyl or C₁₋₄ alkoxy groups.

2. The compound of claim 1, wherein the pharmaceutically acceptable salts include salts formed by the compound represented by the general formula (I) with acids, wherein the acids include inorganic acids, organic acids or acidic amino acids; wherein the inorganic acids include hydrochloric acid, hydrobromic acid, hydrofluoric acid, sulfuric acid, nitric acid or phosphoric acid; the organic acids include formic acid, acetic acid, propionic acid, oxalic acid, trifluoroacetic acid, malonic acid, succinic acid, fumaric acid, maleic acid, lactic acid, malic acid, tartaric acid, citric acid, picric acid, methanesulfonic acid, p-toluenesulfonic acid, ethanesulfonic acid or benzenesulfonic acid; the acidic amino acids include aspartic acid or glutamic acid.

3. The compound of claim 1, wherein the compound corresponds to a compound of formula 1, a compound of formula 2, a compound of formula 3, a compound of formula 4, a compound of formula 5, a compound of formula 6, a compound of formula 7, a compound of formula 8, a compound of formula 9, a compound of formula 10, a compound of formula 11, a compound of formula 12, a compound of formula 13, a compound of formula 14, a compound of formula 15, a compound of formula 16, a compound of formula 17, a compound of formula 18, a compound of formula 19, a compound of formula 20, a compound of formula 21, a compound of formula 22, a compound of formula 23, a compound of formula 24, a compound of formula 25, a compound of formula 26, a compound of formula 27, a compound of formula 28, a compound of formula 29, a compound of formula 30, a compound of formula 31, a compound of formula 32, a compound of formula 33, a compound of formula 34, a compound of formula 35, a compound of formula 36, a compound of formula 37, a compound of formula 38, a compound of formula 39, a compound of formula 40, a compound of formula 41, a compound of formula 42, a compound of formula 43, a compound of formula 44, a compound of formula 45, a compound of formula 46, a compound of formula 47, a compound of formula 48, a compound of formula 49, or a compound of formula 50:

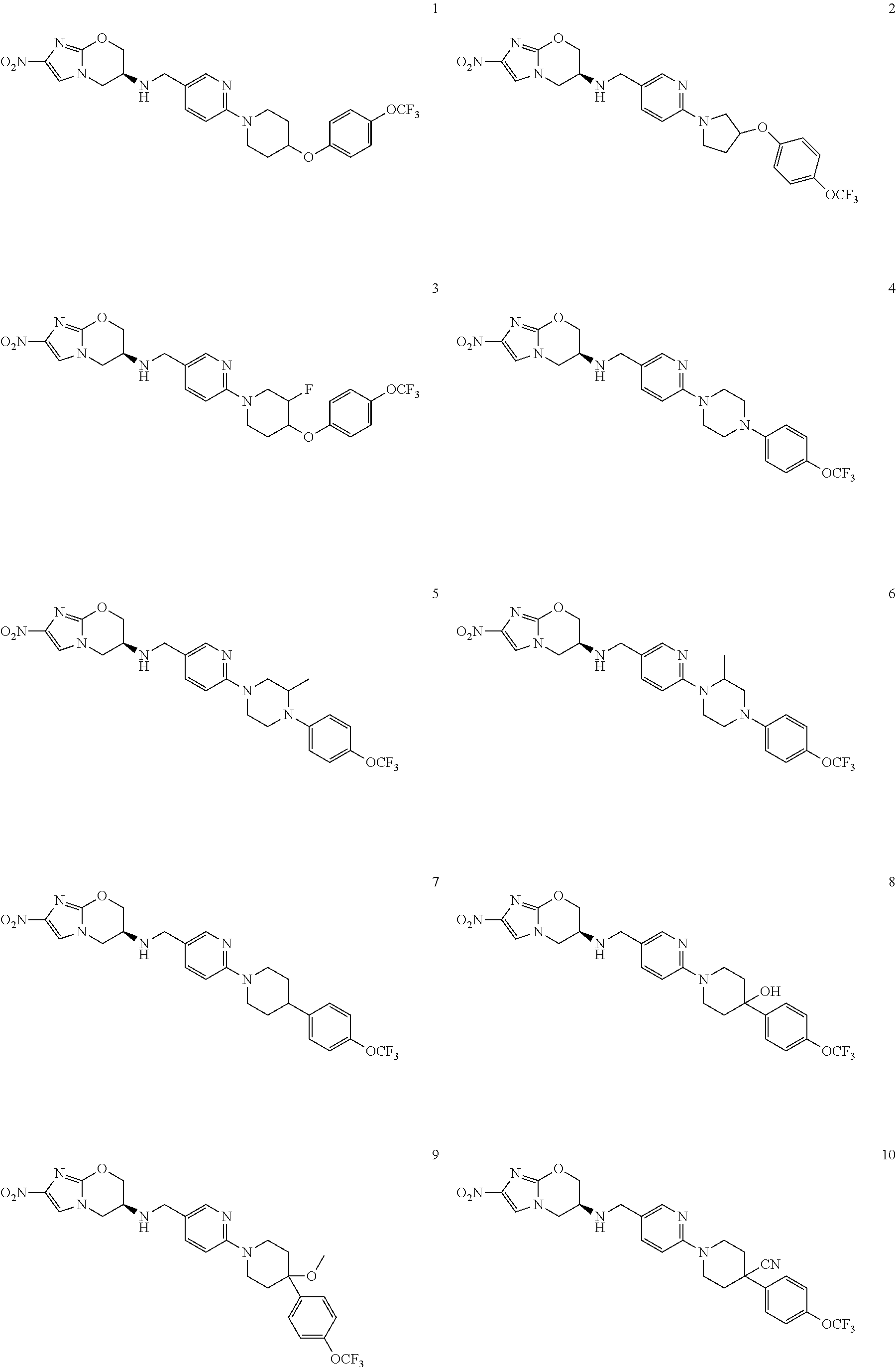

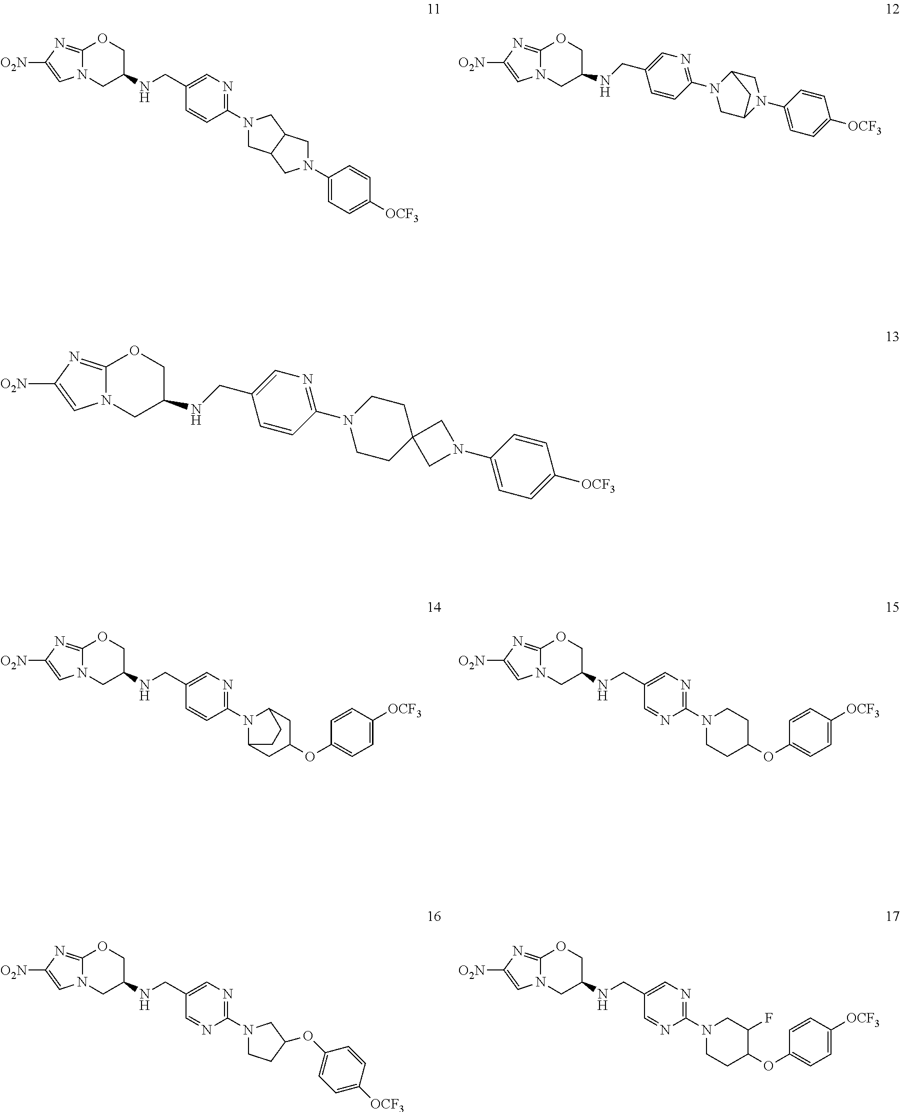

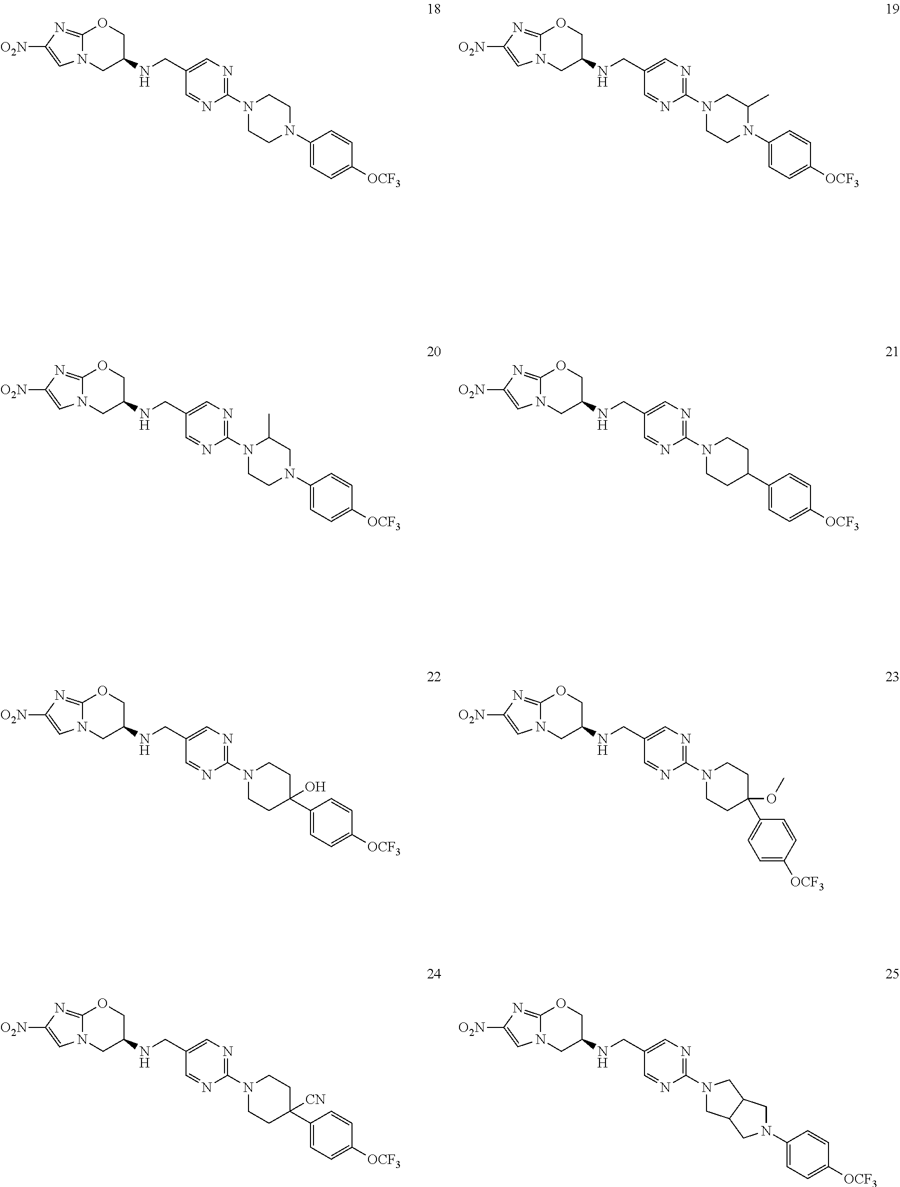

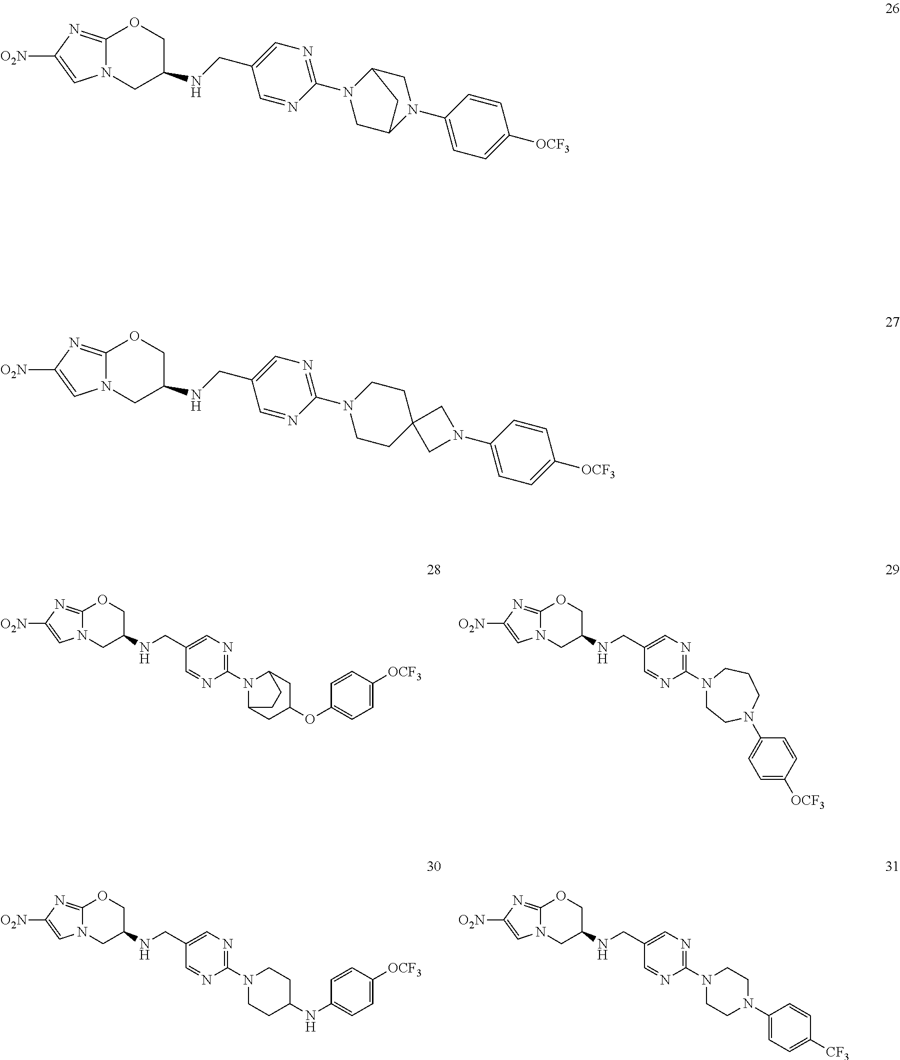 [SMILES: O=[N+]([O-])c1cn2c(n1)OC[C@@H](NCc1cnc(N3CC4CC3CN4c3ccc(OC(F)(F)F)cc3)nc1)C2 ; O=[N+]([O-])c1cn2c(n1)OC[C@@H](NCc1cnc(N3CCC4(CC3)CN(c3ccc(OC(F)(F)F)cc3)C4)nc1)C2 ; O=[N+]([O-])c1cn2c(n1)OC[C@@H](NCc1cnc(N3C4CCC3CC(Oc3ccc(OC(F)(F)F)cc3)C4)nc1)C2 ; O=[N+]([O-])c1cn2c(n1)OC[C@@H](NCc1cnc(N3CCCN(c4ccc(OC(F)(F)F)cc4)CC3)nc1)C2 ; O=[N+]([O-])c1cn2c(n1)OC[C@@H](NCc1cnc(N3CCC(Nc4ccc(OC(F)(F)F)cc4)CC3)nc1)C2 ; O=[N+]([O-])c1cn2c(n1)OC[C@@H](NCc1cnc(N3CCN(c4ccc(C(F)(F)F)cc4)CC3)nc1)C2]

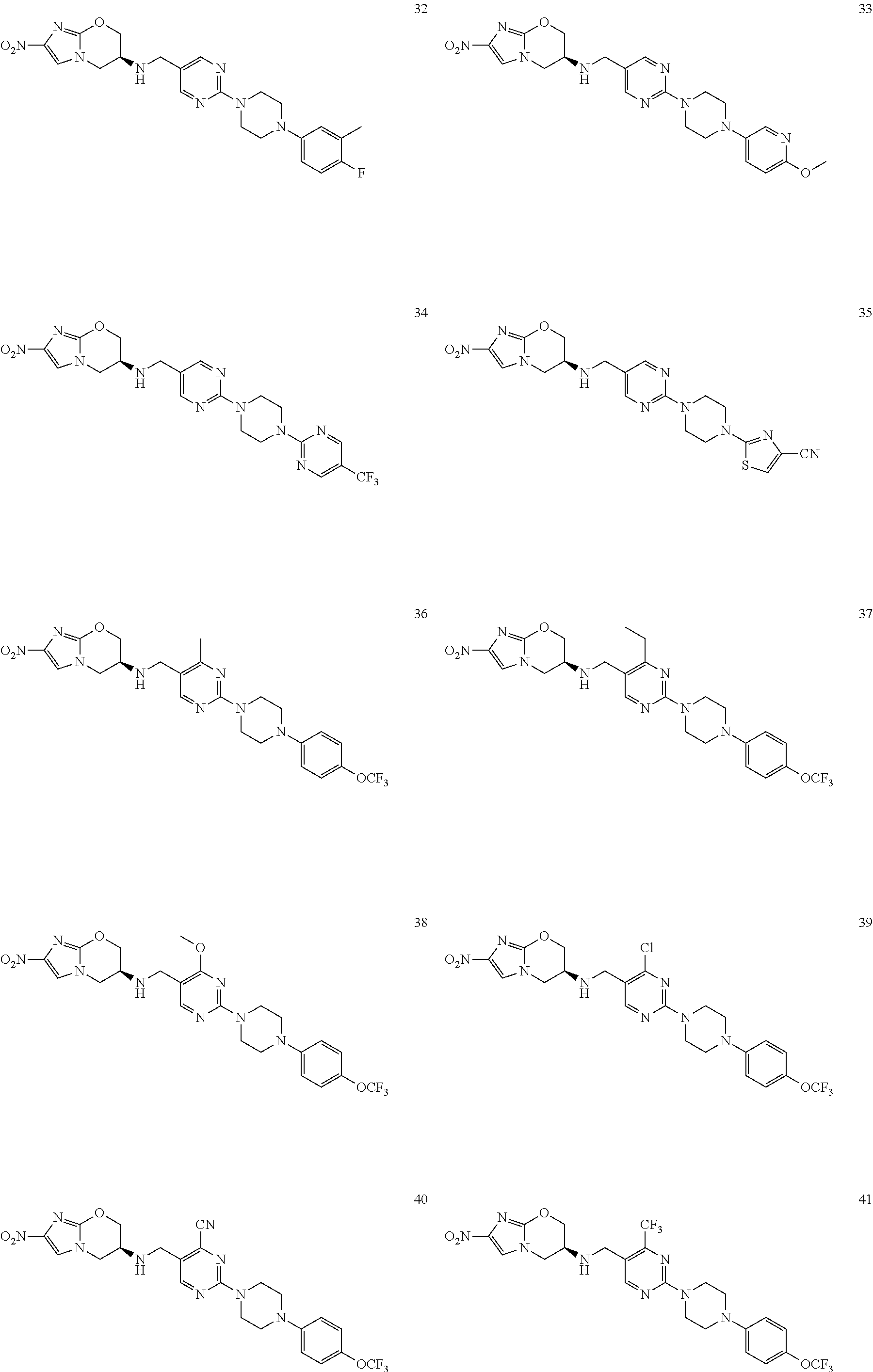

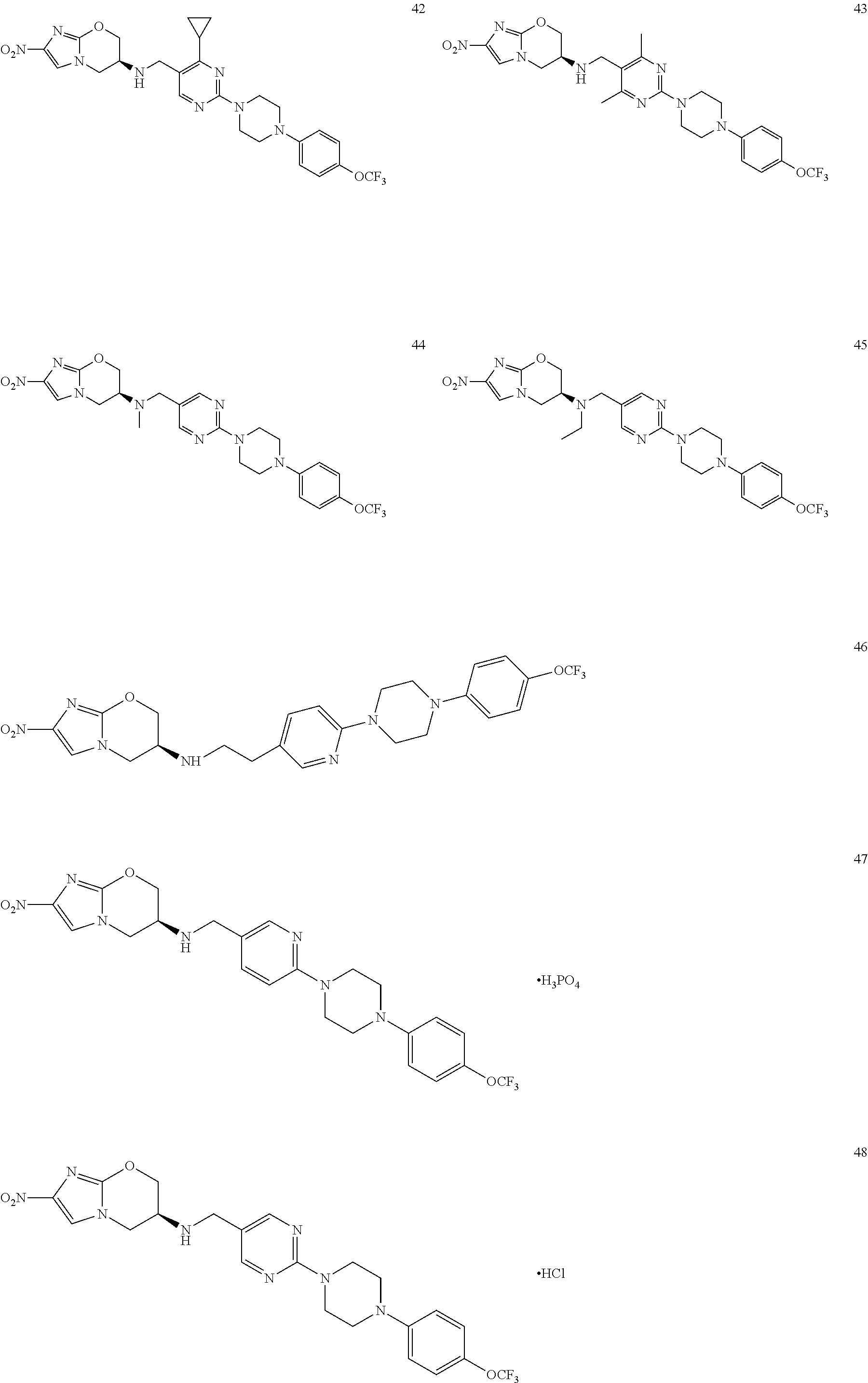

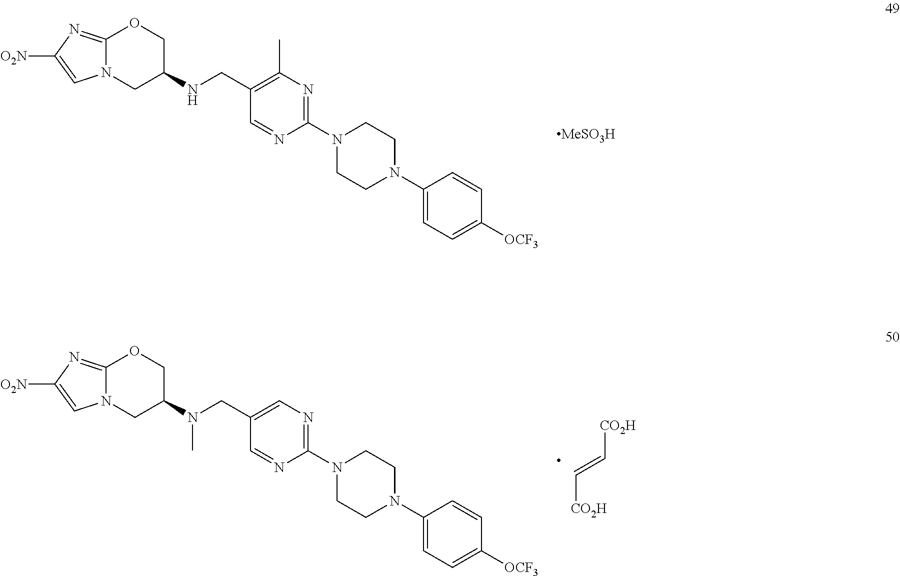

4. A method to produce a nitroimidazole compound of claim 1 with a reaction formula as follows: 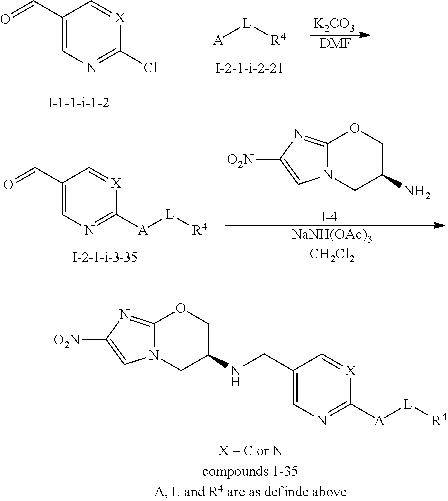
wherein the method comprises the steps of:
(1) subjecting raw materials I-1-1-I-1-2 and I-2-1-I-2-21 to a substitution reaction for 1-24 hours in a solvent at 20° C. to 150° C. or solvent reflux temperature under alkaline conditions, giving intermediates I-3-1-I-3-35, wherein the solvent is selected from one or more of acetonitrile, acetone, dioxane, tetrahydrofuran, methanol, ethanol, isopropanol, dimethylformamide, dimethylacetamide, ethylene glycol dimethyl ether,
dimethylsulfoxide and water; the base is selected from sodium hydroxide, potassium hydroxide, lithium hydroxide, barium hydroxide, potassium carbonate, sodium carbonate, cesium carbonate, sodium bicarbonate, potassium bicarbonate, potassium tert-butoxide, sodium tert-butoxide, sodium hydride, potassium hydride, triethylamine or diisopropylethylamine; and
(2) reacting intermediate I-3-1-I-3-35 with amine 1-4 in a solvent under alkaline conditions to form an imine intermediate state which is then subjected to a reductive amination reaction for 1-24 hours in the presence of a reducing agent, giving compound 1-compound 35, wherein the solvent is selected from one or more of methanol, ethanol, isopropanol, tetrahydrofuran, dichloromethane, 1,2-dichloroethane, dioxane, dimethylformamide, acetonitrile, ethylene glycol dimethyl ether and water; the base is selected from the organic bases including pyridine, triethylamine and diisopropylethylamine, and the reducing agent is selected from sodium borohydride, potassium borohydride, sodium cyanoborohydride or sodium triacetoxyborohydride.

5. A method to produce a nitroimidazole compound of claim 1 with a reaction formula as follows: 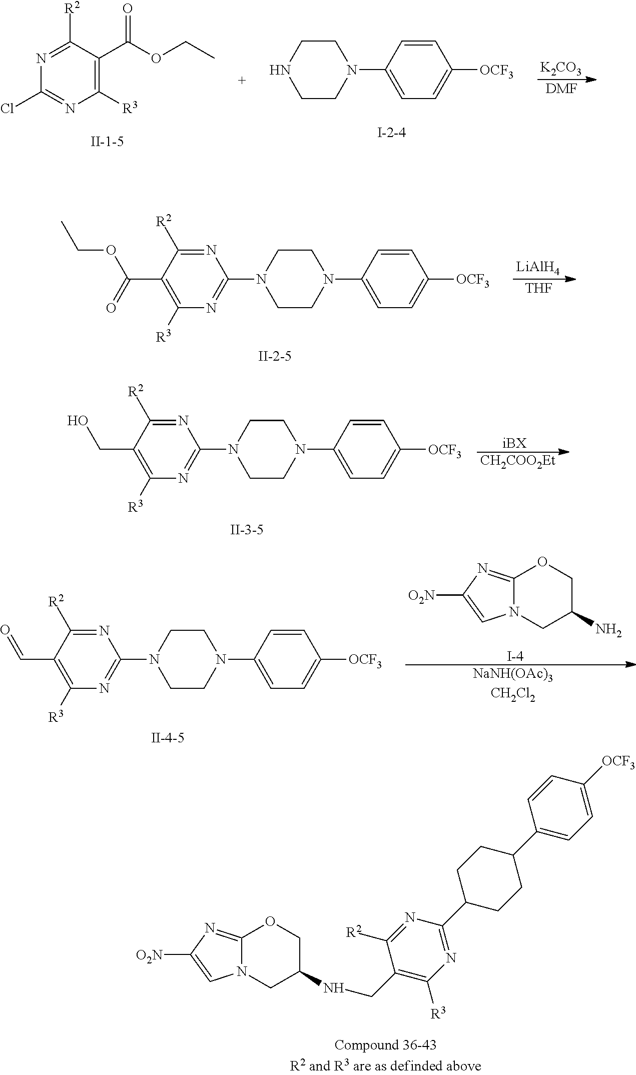
wherein the method comprises the steps of:
(1) subjecting raw materials II-1-1-II-1-8 and I-2-4 to a substitution reaction for 1-24 hours in a solvent at 20° C. to 150° C. or solvent reflux temperature, giving intermediate II-2-1-II-2-8, wherein the solvent is selected from one or more of acetonitrile, acetone, dioxane, tetrahydrofuran, methanol, ethanol, isopropanol, dimethylformamide, dimethylacetamide, ethylene glycol dimethyl ether, dimethylsulfoxide and water; the base is selected from sodium hydroxide, potassium hydroxide, lithium hydroxide, barium hydroxide, potassium carbonate, sodium carbonate, cesium carbonate, sodium bicarbonate, potassium bicarbonate, potassium tert-butoxide, sodium tert-butoxide, sodium hydride, potassium hydride, triethylamine or diisopropylethylamine;
(2) subjecting intermediates II-2-1-II-2-8 to a reduction reaction for 0.5-24 hours in a solvent at −78° C. to 40° C., giving intermediates II-3-1-II-3-8, wherein the solvent is selected from one or more of toluene, tetrahydrofuran, n-hexane, cyclohexane, methyltetrahydrofuran, diethyl ether, methyl tert-butyl ether, ethylene glycol dimethyl ether and water; the reducing agent is selected from sodium borohydride, potassium borohydride, lithium borohydride, lithium aluminum hydride, diisobutylaluminum hydride or red aluminum;
(3) subjecting intermediates II-3-1-II-3-8 to an oxidation reaction for 1-24 hours in a solvent at 20° C.−150° C. or solvent refluxing temperature, giving intermediates II-4-1-II-4-8, wherein the solvent is selected from one or more of ethyl acetate, dichloromethane, dioxane, tetrahydrofuran, trichloromethane, cyclohexane, dimethylformamide, dimethylacetamide, ethylene glycol dimethyl ether and dimethylsulfoxide; the oxidizing agent is selected from active manganese dioxide, 2-iodacyl benzoic acid, Dess-Martin periodinane, pyridinium chlorochromate, pyridinium dichromate, pyridine sulfur trioxide or dimethylsulfoxide and oxalyl chloride; and
(4) reacting intermediates II-4-1-II-4-8 with amine 1-4 in a solvent under alkaline conditions to form an imine intermediate state which is then subjected to a reductive amination reaction for 1-24 hours in the presence of a reducing agent, giving compound 36-compound 43, wherein the solvent is selected from one or more of methanol, ethanol, isopropanol, tetrahydrofuran, dichloromethane, 1,2-dichloroethane, dioxane, dimethylformamide, acetonitrile, ethylene glycol dimethyl ether and water; the base is selected from the organic bases including pyridine, triethylamine and diisopropylethylamine; the reducing agent is selected from sodium borohydride, potassium borohydride, sodium cyanoborohydride or sodium triacetoxyborohydride.

6. A method to produce a nitroimidazole compound of claim 1 with a reaction formula as follows: 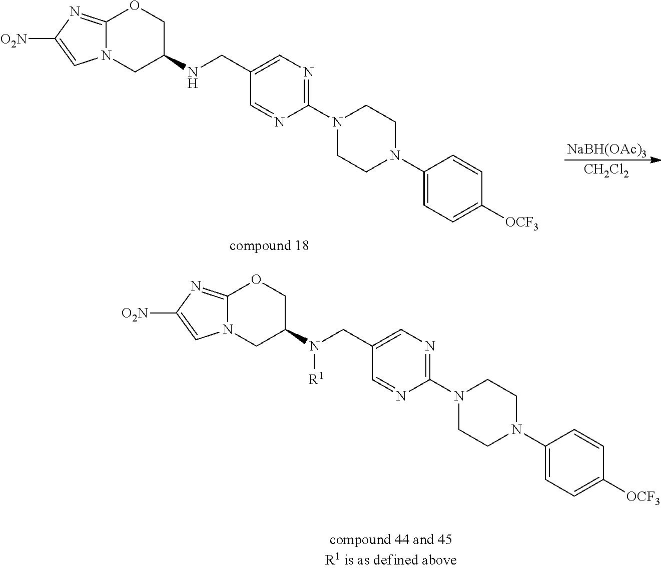
wherein the method comprises the step of: reacting compound 18 with different aldehydes in a solvent under acidic conditions to form an imine intermediate state which is then subjected to a reductive amination reaction for 1-24 hours in the presence of a reducing agent, giving compound 44-compound 45, wherein the solvent is selected from one or more of methanol, ethanol, isopropanol, tetrahydrofuran, dichloromethane, 1,2-dichloroethane, dioxane, dimethylformamide, acetonitrile, ethylene glycol dimethyl ether and water; the acid is an organic weak acid or Lewis acid and selected from acetic acid, zinc chloride, zinc bromide or boron trifluoride diethyl etherate; the reducing agent is selected from sodium borohydride, potassium borohydride, sodium cyanoborohydride or sodium triacetoxyborohydride.

7. A method to produce a nitroimidazole compound of claim 1 with a reaction formula as follows: 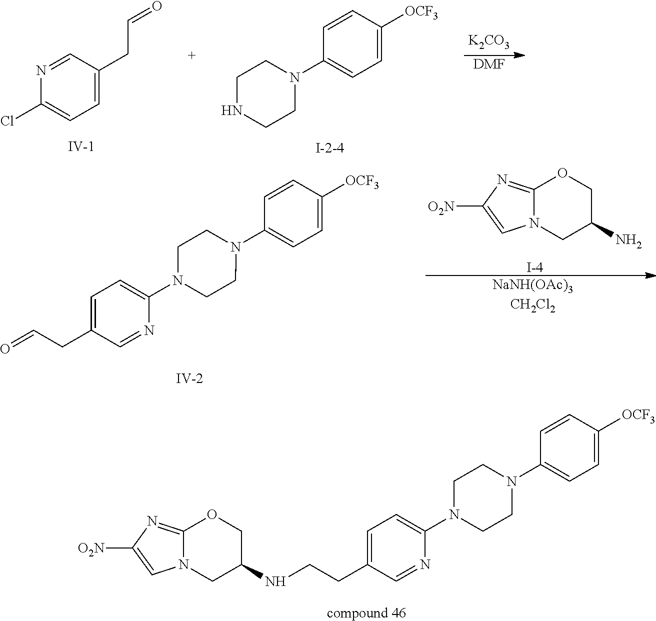
wherein the method comprises the steps of:
(1) subjecting raw materials IV-1 and I-2-4 to a substitution reaction for 1-24 hours in a solvent at 20° C. to 150° C. or solvent refluxing temperature under alkaline conditions, giving intermediate IV-2, wherein the solvent is selected from one or more of acetonitrile, acetone, dioxane, tetrahydrofuran, methanol, ethanol, isopropanol, dimethylformamide, dimethylacetamide, ethylene glycol dimethyl ether, dimethylsulfoxide and water; the base is selected from sodium hydroxide, potassium hydroxide, lithium hydroxide, barium hydroxide, potassium carbonate, sodium carbonate, cesium carbonate, sodium bicarbonate, potassium bicarbonate, potassium tert-butoxide, sodium tert-butoxide, sodium hydride, potassium hydride, triethylamine or diisopropylethylamine; and
(2) reacting intermediate IV-2 with amine I-4 in a solvent under alkaline conditions to form an imine intermediate state which is then subjected to a reductive amination reaction for 1-24 hours in the presence of a reducing agent, giving compound 46, wherein the solvent is selected from one or more of methanol, ethanol, isopropanol, tetrahydrofuran, dichloromethane, 1,2-dichloroethane, dioxane, dimethylformamide, acetonitrile, ethylene glycol dimethyl ether and water; the base is selected from the organic bases including pyridine, triethylamine and diisopropylethylamine; the reducing agent is selected from sodium borohydride, potassium borohydride, sodium cyanoborohydride or sodium triacetoxyborohydride.

8. A method to produce a nitroimidazole compound of claim 1 with a reaction formula as follows: 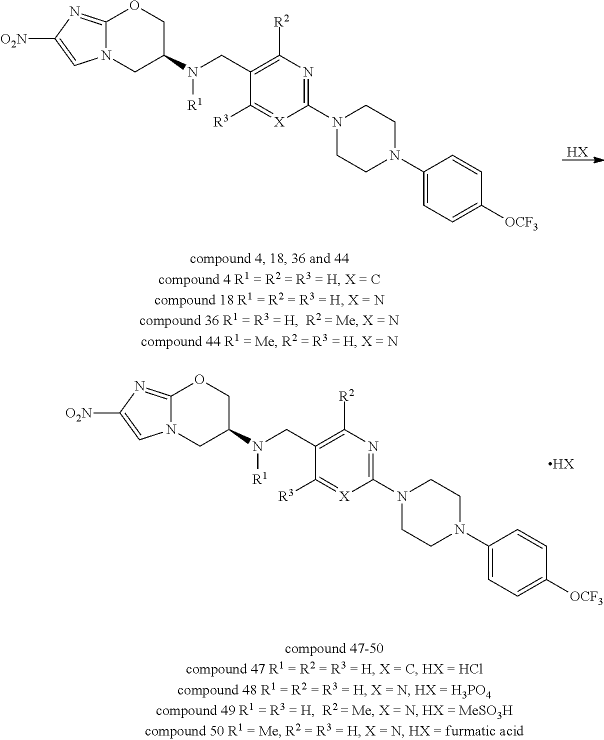
wherein the method comprises the steps of: respectively reacting compound 4 with hydrochloric acid, compound 18 with phosphoric acid, compound 36 with methanesulfonic acid and compound 44 with fumaric acid for 1-48 hours in a solvent under conditions of −20° C. to 100° C. for direct precipitation of solids or static precipitation of solids or concentration and recrystallization, giving compound 47-compound 50, wherein the solvent is selected from one or more of acetone, tetrahydrofuran, acetonitrile, ethanol, methanol, isopropanol, dichloromethane, 1,4-dioxane, dimethylformamide, dimethylacetamide, N-methylpyrrolidone,
dimethylsulfoxide or water.

9. (canceled)

10. A pharmaceutical composition for treating infectious diseases caused by Mycobacterium tuberculosis, comprising a therapeutically effective amount of a nitroimidazole compound of claim 1 and pharmaceutically acceptable excipients or carriers.

11. A method of treating an infectious diseases caused by Mycobacterium tuberculosis comprising administering to a mammal in need thereof a composition comprising a therapeutically effective amount of a compound of claim 1.
